(19) United States
(12) Patent Application Publication
(10) Pub. No.: US 2025/0388645 A1
(43) Pub. Date: Dec. 25, 2025

(54) Immune Cells Comprising Truncated NKG2D Chimeric Receptors

(71) Applicants: National University of Singapore, Singapore (SG); Nkarta, Inc., South San Francisco, CA (US)

(72) Inventors: Jun Hao Leong, Singapore (SG); Noriko Shimasaki, Singapore (SG); See Voon Seow, Singapore (SG); Dario Campana, Singapore (SG); James Barnaby Trager, Albany, CA (US); Alexandra Leida Liana Lazetic, San Jose, CA (US); Chao Guo, San Francisco, CA (US); Luxuan Guo Buren, San Francisco, CA (US); Shyam Sashikant Masrani, London (GB)

(21) Appl. No.: 19/227,964

(22) Filed: Jun. 4, 2025

Related U.S. Application Data
(63) Continuation of application No. 17/740,899, filed on May 10, 2022, now Pat. No. 12,351,617.
Application No. 17/740,899 is a continuation of application No. 16/497,678, filed on Sep. 25, 2019, now Pat. No. 11,365,236.
(60) Provisional application No. 62/477,335, filed on Mar. 27, 2017.
(60) Provisional application No. 62/628,774, filed on Feb. 9, 2018.

(51) Int. Cl. C07K 14/705; A61K 40/15; A61K 40/31; A61K 40/42; C07K 14/54; C07K 14/725; C12N 15/62

(52) U.S. Cl. CPC C07K 14/7056; A61K 40/15; A61K 40/31; A61K 40/4224; C07K 14/5443; C07K 14/7051; C07K 14/70517; C12N 15/625; C07K 14/70578; C07K 2317/24; C07K 2317/53; C07K 2319/02; C07K 2319/03; C07K 2319/33

(57) ABSTRACT

Several embodiments disclosed herein relate to the compositions comprising engineered Natural Killer (NK) cells that express a chimeric receptor, the chimeric receptor imparting to the NK cells an enhanced ability to target specific cells, such as cancerous cells or those affected by an infectious disease. Several embodiments relate to NK cells that target cells expressing natural ligands of NKG2D, where the NK cells comprise transmembrane and/or signaling domains that lead to cytotoxic and/or cytolytic effects when the NK cells bind a target cell. Uses of NK cell compositions to treat diseases are also provided for in several embodiments.

### RELATED APPLICATIONS

This application is a continuation of U.S. application Ser. No. 17/740,899, filed on May 10, 2022, which is a continuation of U.S. application Ser. No. 16/497,678, filed on Mar. 27, 2018, now granted U.S. Pat. No. 11,365,236, which is the U.S. National Stage of International Application No. PCT/US2018/024650, filed on Mar. 27, 2018, which designates the U.S., published in English, and claims the benefit of U.S. Provisional Application No. 62/477,335, filed on Mar. 27, 2017 and U.S. Provisional Application No. 62/628,774, filed on Feb. 9, 2018. The entire teachings of the above applications are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 44591144031.xml; created Sep. 17, 2025, 203,936 Bytes in size.

### BACKGROUND

The emergence and persistence of many diseases is characterized by an insufficient immune response to aberrant cells, including malignant and virally infected cells. Immunotherapy is the use and manipulation of the patient's immune system for treatment of various diseases.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells to achieve the targeted recognition and destruction of aberrant cells of interest.

To address this need for specifically targeting and destroying, disabling or otherwise rendering inert diseased or infected cells, there are provided for herein polynucleotides, amino acids, and vectors that encode chimeric receptors that impart enhanced targeting and cytotoxicity to cells, such as natural killer cells. Also provided for are methods for producing the cells, and methods of using the cells to target and destroy diseased or damaged cells. In several embodiments, there is provided a polynucleotide encoding a chimeric receptor comprising an extracellular receptor domain and an effector domain comprising a transmembrane region and an intracellular signaling domain, wherein the extracellular receptor domain comprises a peptide that binds native ligands of Natural Killer Group 2 member D (NKG2D), wherein the peptide that binds native ligands of NKG2D is a fragment of NKG2D.

In several embodiments, there is provided a polynucleotide encoding a chimeric receptor comprising one or both of: (a) an extracellular receptor domain and (b) an effector domain comprising a transmembrane region and an intracellular signaling domain. In several embodiments, the extracellular receptor domain comprises a peptide that binds native ligands of Natural Killer Group 2 member D (NKG2D). In several embodiments, the peptide that binds native ligands of NKG2D is a fragment of NKG2D, for example, a fragment of NKG2D is encoded by a polynucleotide comprising SEQ ID NO. 2. As disclosed, herein, additional NKG2D fragments are also used, depending on the embodiment. In several embodiments, the intracellular signaling domain comprises CD3zeta. In one embodiment, the CD3zeta is encoded by a polynucleotide comprising SEQ ID NO. 13, though, as disclosed herein, sequences that differ from CD3zeta, but share similar function may also be used, depending on the embodiment.

In several embodiments, the transmembrane region of the effector domain comprises a CD8α transmembrane domain. In one embodiment, the transmembrane region of the effector domain further comprises a CD8α hinge region. In several embodiments, the CD8α hinge region is encoded by a polynucleotide comprising SEQ ID NO: 5. In several embodiments, the intracellular signaling domain further comprises 4-1BB. In one embodiment, the 4-1BB is encoded by a polynucleotide comprising SEQ ID NO. 12, though, as disclosed herein, sequences that differ from 4-1BB, but share similar function may also be used, depending on the embodiment.

In several embodiments, the chimeric receptor comprises the fragment of NKG2D coupled to CD8α, 4-1BB and CD3zeta. In several embodiments, such a chimeric receptor is encoded by the nucleic acid sequence of SEQ ID NO. 18. In additional embodiments, the chimeric receptor is encoded by the nucleic acid sequence of SEQ ID NO. 108, though, as disclosed herein, sequences that differ from SEQ ID NO. 108, but share similar function may also be used, depending on the embodiment. In several embodiments, the chimeric receptor comprises the amino acid sequence of SEQ ID NO. 19.

In several embodiments, any of chimeric receptors disclosed herein can also be co-expressed with membrane-bound interleukin 15 (mbIL15). In some embodiments, the mbIL15 is encoded by a polynucleotide comprising SEQ ID NO. 16. In some embodiments, the mbIL15 comprises an amino acid sequence of SEQ ID NO: 17. Other sequences for mbIL15 may also be used, depending on the embodiment. In some embodiments, the mbIL15 is bicistronically expressed on the same polynucleotide as the chimeric receptor. In other embodiments, the mbIL15 is co-expressed on a separate construct. In several embodiments, the intracellular signaling domain is further enhanced by coupling its expression with that of membrane-bound interleukin 15 (mbIL15).

In several embodiments, the effector domain further comprises an OX-40 domain. In several embodiments, the OX-40 domain is either in place of, or in addition to mbIL15. In several embodiments, the chimeric receptor comprises the fragment of NKG2D coupled to a CD8α hinge, a CD8α transmembrane domain, the OX-40 domain, and the CD3zeta. In some embodiments, the polynucleotide construct is configured to bicistronically co-express mbIL15. In some such embodiments, the polynucleotide construct comprises one or more cleavage sites (e.g., T2A, P2A, E2A, and/or F2A cleavage site(s)) recognized and cleaved by, for example, a cytosolic protease. In some embodiments, the mbIL15 is coupled to the chimeric receptor by a cytosolic protease cleavage site. In several embodiments, the chimeric receptor is encoded by the nucleic acid sequence of SEQ ID NO: 90 coupled to the mbIL15 encoded by SEQ ID NO. 16 by a cytosolic protease cleavage site. In several embodiments, the chimeric receptor is encoded by the nucleic acid sequence of SEQ ID NO: 109 coupled to the mbIL15 encoded by SEQ ID NO. 16 by a cytosolic protease cleavage site. In several embodiments, the chimeric receptor comprises the amino acid sequence of SEQ ID NO: 91 and is co-expressed with mbIL15 comprising the amino acid sequence of SEQ ID NO. 17. As disclosed herein, sequences that differ from SEQ ID NOs: 90, 91, 109, 16, and/or 16, but share similar function may also be used, depending on the embodiment.

In several embodiments, the chimeric receptor comprises the fragment of NKG2D coupled to a IgG4 hinge, a CD8α transmembrane domain, the OX-40 domain, and the CD3zeta. In some embodiments, the polynucleotide construct is configured to bicistronically co-express mbIL15 with the chimeric receptor. In some such embodiments, the polynucleotide construct comprises one or more cleavage sites (e.g., T2A, P2A, E2A, and/or F2A cleavage site(s)) recognized and cleaved by a cytosolic protease. In some embodiments, the mbIL15 is coupled to the chimeric receptor by a cytosolic protease cleavage site. In several embodiments, the chimeric receptor is encoded by the nucleic acid sequence of SEQ ID NO: 100 coupled to the mbIL15 encoded by SEQ ID NO. 16 by a cytosolic protease cleavage site. In several embodiments, the chimeric receptor comprises the amino acid sequence of SEQ ID NO: 101 and is co-expressed with mbIL15 comprising the amino acid sequence of SEQ ID NO. 17. As disclosed herein, sequences that differ from SEQ ID NOs: 100, 101 and/or 16, but share similar function may also be used, depending on the embodiment.

In several embodiments, there are provided methods for treating cancer, comprising administering to a subject having a cancer a composition comprising a Natural Killer (NK) cell expressing the chimeric receptor encoded by the polynucleotides described above, or elsewhere herein.

In one embodiment, the NK cells are autologous cells isolated from a patient having a cancer or an infectious disease. In additional embodiments, the NK cells are allogeneic cells isolated from a donor.

Also provided for herein is use of a polynucleotide as described above, or elsewhere herein, in the manufacture of a medicament for enhancing NK cell cytotoxicity in a mammal in need thereof. In several embodiments, there is provided for the use of a polynucleotide as described above, or elsewhere herein, in the manufacture of a medicament for treating or preventing cancer or an infectious disease in a mammal in need thereof.

According to several embodiments, there is provided a polynucleotide encoding a chimeric receptor, the chimeric receptor comprising an extracellular receptor domain an effector domain comprising a transmembrane region and an intracellular signaling domain. As discussed in more detail herein, the extracellular receptor domain serves to recognize and bind ligands on a target cell. The effector domain serves to transmit signals (upon binding of a target cell by the extracellular domain) that set in motion a signal cascade that leads to cytotoxic activity against the target cell. In accordance with several embodiments, the polynucleotide encodes a chimeric receptor that provides unexpectedly increased cytotoxicity as compared to non-engineered NK cells.

In several embodiments, the extracellular receptor domain comprises a peptide that binds native ligands of Natural Killer Group 2 member D (NKG2D). According to several embodiments, the peptide that binds native ligands of NKG2D is a functional fragment of NKG2D (e.g., a truncation, fragment or portion of full length NKG2D. As used, herein the terms, “fragment”, “truncation”, and “portion” shall be given their ordinary meanings and shall also be interchangeable with one another. For example, in several embodiments, the fragment of NKG2D is encoded by a polynucleotide comprising a fragment of the sequence of SEQ ID NO: 1. In several embodiments, the fragment of NKG2D comprises the sequence of SEQ ID NO: 2, while in additional embodiments, the fragment encoding NKG2D is codon optimized, and comprises, for example, the sequence of SEQ ID NO: 3. In additional embodiments, the fragment encoding NKG2D is codon optimized, and comprises, for example, the sequence of SEQ ID NO: 68.

In several embodiments, the effector domain comprises one or more of CD16, NCR1, NCR2, NCR3, 4-1BB, NKp80, CD3zeta and 2B4. In several embodiments, these effector domains are coupled to CD8α.

In several embodiments, the chimeric receptor comprises a fragment of NKG2D coupled to CD16. As used herein, coupled shall be given its ordinary meaning and shall also refer to direct (e.g., a first nucleotide followed directly be a second nucleotide) or indirect (e.g., sequences are in frame with one another but separated by intervening nucleotides) linkage of nucleotide sequences in a manner that allows for expression of the nucleotide sequences in, for example, an in vitro transcription/translation system, a host cell (e.g., in vitro and/or in vivo). As used herein, “linked” and “coupled” are used interchangeably. In several embodiments, the NKG2D/CD16 chimeric receptor is encoded by the nucleic acid sequence of SEQ ID NO: 23. In several embodiments, the NKG2D/CD16 chimeric receptor comprises the amino acid sequence of SEQ ID NO: 24. In several embodiments, the chimeric receptor comprises a fragment of NKG2D coupled to NCR1. In several embodiments, such a chimeric receptor is encoded by the nucleic acid sequence of SEQ ID NO: 27. In several embodiments, the chimeric receptor comprises the amino acid sequence of SEQ ID NO: 28.

As discussed above, in several embodiments, the NKG2D fragment is coupled to NCR2, and the resultant chimeric receptor comprises at least a portion of the amino acid sequence of SEQ ID NO: 21. Several embodiments provide for a chimeric receptor comprising a fragment of NKG2D coupled to NCR3. In several embodiments, the chimeric receptor is encoded by the nucleic acid sequence of SEQ ID NO. 29, and the chimeric receptor comprises the amino acid sequence of SEQ ID NO. 30.

As discussed in more detail below, combinations of transmembrane and intracellular domains are used in several embodiments and provide for synergistic interactions between the components of the chimeric receptor and yield enhanced cytotoxic effects. In several embodiments, the chimeric receptor comprises the fragment of NKG2D coupled to a CD16 transmembrane/intracellular domain and 4-1BB. In several embodiments, the chimeric receptor comprises the fragment of NKG2D coupled to a CD8α hinge, a CD16 transmembrane/intracellular domain and 4-1BB. In several embodiments, such a chimeric receptor is encoded by the nucleic acid sequence of SEQ ID NO: 25. In several embodiments, the resultant chimeric receptor comprises the amino acid sequence of SEQ ID NO: 26.

In several embodiments, NCR1 is used in conjunction with the NKG2D fragment. In several embodiments, the NKG2D fragment is linked to NCR1 alone. In additional embodiments, the chimeric receptor comprises the fragment of NKG2D coupled to NCR1 and 4-1BB. In some such embodiments, the chimeric receptor comprises the NCR1 amino acid sequence of SEQ ID NO: 20.

In several embodiments, the chimeric receptor comprises the fragment of NKG2D coupled to CD8α, 4-1BB and CD3zeta. In several embodiments, such an NKG2D/CD8α/4-1BB/CD3zeta chimeric receptor is encoded by the nucleic acid sequence of SEQ ID NO. 18. In several embodiments, the chimeric receptor comprises the amino acid sequence of SEQ ID NO. 19.

In several embodiments, NCR3 is included in the chimeric receptor. For example, an NKG2D/NCR3 construct is provided for in several embodiments. The resultant chimeric receptor thereby comprises the NCR3 amino acid sequence of SEQ ID NO: 22. In several embodiments, the chimeric receptor comprises a NKG2D/NCR2/4-1BB construct or an NKG2D/NCR3/4-1BB construct.

In several embodiments, linkers, hinges, or other “spacing” elements are provided for in the chimeric receptor constructs. For example, in several embodiments, the effector domain comprises a linker. In several embodiments, the polynucleotides encode a GS linker between the portions of the construct, such as between any of 4-1BB, CD16, NCR1, NCR3, 2B4 or NKp80. In several embodiments, one or more GS linkers are provided for, for example, 1, 2, 3, 4, 5, 6, or more. In several embodiments, there is provided for a chimeric receptor comprising a hinge region. Depending on the location within a particular construct, a hinge region can be synonymous with a linker region, and vice versa. In several embodiments, the hinge region is encoded by the nucleic acid sequence of SEQ ID NO: 5. In some embodiments, the hinge region can be truncated to a desired length and is therefore encoded by a fragment of the nucleic acid sequence of SEQ ID NO: 5. In several embodiments, a glycine-serine motif is used as a hinge. In several embodiments, the hinge region is comprises a glycine-serine repeating motif having the amino acid sequence of (GGGGS)n (SEQ ID NO: 31) where n is the number of repeats. In several embodiments, 9 repeats are used, resulting in a hinge region comprising the amino acid sequence of SEQ ID NO: 33. In several embodiments, 3 repeats are used, resulting in a hinge region comprising the amino acid sequence of SEQ ID NO: 34.

In several embodiments, two separate molecules can be used as a hinge or linker, such as the amino acid sequence of SEQ ID NO: 32 (CD8α/GS3). In several embodiments, portions of a beta adrenergic receptor are used as a hinge or linker. In several embodiments, portions of the beta-2 adrenergic receptor are used. In one embodiment, an extracellular domain of the beta-2 adrenergic receptor is used, which is encoded by the nucleic acid sequence of SEQ ID NO: 40. In some embodiments, the first transmembrane helix of the beta-2 adrenergic receptor is used, which is encoded by the nucleic acid sequence of SEQ ID NO: 42. Depending on the embodiment, these two beta-2 adrenergic receptor portions are used together in the chimeric receptor. In several embodiments, the extracellular receptor domain further comprises a CD8α signal peptide, wherein the signal peptide comprises the nucleic acid sequence of SEQ ID NO. 4. Other signal peptides are optionally used, depending on the embodiment. Signal peptides may be employed in a multimeric format, according to some embodiments.

In several embodiments, the effector domain comprises one or more hemi-ITAM sequences. In some such embodiments, the hemi-ITAM comprises the amino acid motif DGYXXL (where X is any amino acid; SEQ ID NO: 14). Multiple hemi-ITAMs are used in some embodiments. In several embodiments, the hemi-ITAM comprises NKp80. In several embodiments, the effector domain comprises one or more ITSM sequences. ITSM sequences are used in conjunction with hemi-ITAM motifs in several embodiments. In several embodiments, the ITSM comprises the amino acid motif S/TXYXXL/I (where X is any amino acid; SEQ ID NO. 15). In several embodiments, the effector comprises a 2B4 domain.

In several embodiments, the chimeric receptor comprises a fragment of NKG2D coupled to a GS3 linker, a CD8α hinge, a CD16 transmembrane/intracellular domain and 4-1BB. In several embodiments, the chimeric receptor comprises a fragment of NKG2D coupled to a GS3 linker, a CD16 transmembrane/intracellular domain and 4-1BB. In several embodiments, the chimeric receptor comprises a fragment of NKG2D coupled to a CD16 transmembrane/intracellular domain and 4-1BB. In several embodiments, the chimeric receptor comprises a fragment of NKG2D coupled to a CD8α hinge, a CD8α transmembrane domain, 4-1BB, and 2B4. In several embodiments, the chimeric receptor comprises a fragment of NKG2D coupled to a beta-adrenergic extracellular domain, a beta-adrenergic transmembrane domain, 4-1BB, and 2B4. In several embodiments, the chimeric receptor comprises a fragment of NKG2D coupled to a CD8α hinge, a CD8α transmembrane domain, 4-1BB, 2B4, a GS3 linker, and NKp80. In several embodiments, the chimeric receptor comprises a fragment of NKG2D coupled to a CD8α hinge, a CD8α transmembrane domain, 4-1BB, a GS3 linker, and NKp80. In several embodiments, the chimeric receptor comprises a fragment of NKG2D, wherein the fragment is encoded by a sequence that is codon optimized coupled to a GS3 linker, an additional NKG2D fragment, a beta-adrenergic extracellular domain, a beta-adrenergic transmembrane domain, 4-1BB, an additional GS3 linker, and NKp80. In several embodiments, the chimeric receptor comprises a fragment of NKG2D that is codon optimized coupled to a GS3 linker, an additional NKG2D fragment, a CD8α hinge, a CD8α transmembrane domain, 4-1BB, an additional GS3 linker, and NKp80. In several embodiments, the chimeric receptor comprises a fragment of NKG2D that is codon optimized coupled to a GS3 linker, an additional NKG2D fragment, a CD8α hinge, a CD16 transmembrane/intracellular domain, and 4-1BB. In several embodiments, chimeric receptor comprises a fragment of NKG2D coupled to a CD8α hinge, a CD16 transmembrane/intracellular domain, 4-1BB, and 2B4. In several embodiments, the chimeric receptor comprises a fragment of NKG2D coupled to a CD8α hinge, a CD16 transmembrane/intracellular domain, 4-1BB, a GS3 linker, and NKp80. In several embodiments, the chimeric receptor comprises a fragment of NKG2D that is coupled to a CD8α hinge and a CD8α transmembrane domain. In several embodiments, the effector comprises 4-1BB. In some such embodiments the effector comprises 4-1BB optionally in conjunction with one or more of NKp80, 2B4, CD3zeta, Dap10, Dap12, CD28, or other signaling domains provided for herein). In several embodiments, the effector domain further comprises CD3zeta. In several embodiments, the effector domain comprises an intracellular domain of 2B4. In several embodiments, the effector domain further comprises an intracellular domain of DAP10.

In several embodiments, the chimeric receptor comprises a fragment of NKG2D coupled to a CD8α hinge, a CD8α transmembrane domain, 4-1BB, 2B4, and CD3zeta. In several embodiments, the chimeric receptor is encoded by the nucleic acid sequence of SEQ ID NO: 58. In several embodiments, the chimeric receptor comprises the amino acid sequence of SEQ ID NO: 59.

Additionally, any of chimeric receptors disclosed herein can also be co-expressed with membrane-bound interleukin 15 (mbIL15). For example, provided for in several embodiments is a polynucleotide encoding a chimeric receptor comprising an extracellular receptor domain, wherein the extracellular receptor domain comprises a peptide that binds native ligands of NKG2D, wherein the peptide that binds native ligands of NKG2D is a fragment of NKG2D, a transmembrane region, an effector domain, the polynucleotide being co-expressed with an additional construct encoding membrane-bound interleukin 15 (mbIL15). In several embodiments, chimeric receptors as discussed herein are co-expressed with mbIL-15. In several embodiments, the effector domain comprises 4-1BB and CD3 zeta, and the transmembrane region comprises CD8α.

In several embodiments, the chimeric receptors are engineered such that they do not include DNAX-activating protein 10 (DAP10). Additionally, in several embodiments, the chimeric receptors are engineered such that they do not include an ITAM motif.

In several embodiments, there is provided a polynucleotide encoding a chimeric receptor comprising, one, two, or all of: (a) an extracellular receptor domain comprising a fragment of NKG2D that binds native ligands of NKG2D, (b) a transmembrane region, wherein the transmembrane region comprises CD8α, and (c) an effector domain, wherein the effector domain comprises 4-1BB and the intracellular domain of 2B4 or DAP10. In several embodiments, the effector domain comprises 2B4 followed by 4-1BB. In additional embodiments, the effector domain comprises 4-1BB followed by 2B4. In several embodiments, the effector domain comprises DAP10 followed by 4-1BB. In additional embodiments, the effector domain comprises 4-1BB followed by DAP10. In several embodiments, the chimeric receptor comprises the fragment of NKG2D coupled to a CD8α hinge, a CD8α transmembrane domain, 4-1BB, and DAP10. In several embodiments, the chimeric receptor is encoded by the nucleic acid sequence of SEQ ID NO: 60. In several embodiments, the chimeric receptor comprises the amino acid sequence of SEQ ID NO: 61. In several embodiments, the chimeric receptor comprises the fragment of NKG2D coupled to a CD8α hinge, a CD8α transmembrane domain, 4-1BB, 2B4, and DAP10. In several embodiments, the effector domain comprises 4-1BB, followed by DAP10, followed by 2B4. In several embodiments, the chimeric receptor is encoded by the nucleic acid sequence of SEQ ID NO: 62. In several embodiments, the chimeric receptor comprises the amino acid sequence of SEQ ID NO: 63. In several embodiments, the effector domain comprises 4-1BB, followed by 2B4, followed by DAP10. In several embodiments, the chimeric receptor is encoded by the nucleic acid sequence of SEQ ID NO: 64. In several embodiments, the chimeric receptor comprises the amino acid sequence of SEQ ID NO: 65.

In several embodiments, the chimeric receptor comprises a codon-optimized fragment of NKG2D coupled to an intracellular effector domain. In several embodiments, multiple fragments of NKG2D are employed, for example, an additional NKG2D fragment (optionally codon optimized) is coupled to the first fragment by, for example, a GS3 linker. In several embodiments, such chimeric receptors further comprise a CD8α hinge, a CD8α transmembrane domain, 4-1BB, and CD3zeta. In several embodiments, the chimeric receptor is encoded by the nucleic acid sequence of SEQ ID NO: 66. In several embodiments, the chimeric receptor comprises the amino acid sequence of SEQ ID NO: 67. In several embodiments, the polynucleotide is co-expressed with an additional construct encoding membrane-bound interleukin 15 (mbIL15).

In several embodiments, there is provided a polynucleotide encoding a chimeric receptor comprising an extracellular receptor domain, comprising a fragment of NKG2D that binds a native ligand of NKG2D and is encoded by a fragment of SEQ ID NO: 1, a transmembrane region comprising a CD3zeta transmembrane region, and an effector domain. In several embodiments, there is provided a polynucleotide encoding a chimeric receptor comprising an extracellular receptor domain, comprising a fragment of NKG2D that binds a native ligand of NKG2D and is encoded by SEQ ID NO. 2, a transmembrane region comprising a CD3zeta transmembrane region, and an effector domain. In several embodiments, there is provided a polynucleotide encoding a chimeric receptor comprising an extracellular receptor domain, comprising a fragment of NKG2D that binds a native ligand of NKG2D and is encoded by SEQ ID NO. 3, a transmembrane region comprising a CD3zeta transmembrane region, and an effector domain. In several embodiments, there is provided a polynucleotide encoding a chimeric receptor comprising an extracellular receptor domain, comprising a fragment of NKG2D that binds a native ligand of NKG2D and is encoded by SEQ ID NO. 68, a transmembrane region comprising a CD3zeta transmembrane region, and an effector domain. In several embodiments, fragments of the NKG2D encoded by any of SEQ ID NO. 2, 3, or 68 may also be used. In several embodiments, the CD3zeta transmembrane region comprises the amino acid sequence of SEQ ID NO: 69. Fragments of the sequence of SEQ ID NO: 69 are also use, in several embodiments, the fragments retaining the ability to transduce at least about 65%, about 75%, about 85%, or about 95% of the signal transduction of a native CD3 zeta subunit (including dimers). In several embodiments, the extracellular receptor domain further comprises additional resides adjacent to the CD3zeta transmembrane region. In several embodiments, the additional amino acids are extracellular residues of a native CD3zeta sequence. In other embodiments, the additional amino acids are randomly selected. In several embodiments, there are 2, 3, 4, 5, 6, 8, 10, 15, or 20 additional amino acids. In several embodiments, the chimeric receptor domain comprises a hinge region, which in several embodiments, a CD8α hinge encoded by the nucleic acid sequence of SEQ ID NO: 5. In several embodiments, the hinge region is a CD8α hinge encoded by a fragment of the nucleic acid sequence of SEQ ID NO: 5. Depending on the embodiment, the fragment is about 75%, about 80%, about 85%, about 90%, about 95% of the length of the nucleic acid sequence of SEQ ID NO: 5. Depending on the embodiment, the fragment is about 75%, about 80%, about 85%, about 90%, about 95%, about 98%, or about 99% homologous to the nucleic acid sequence of SEQ ID NO: 5. In several embodiments, the extracellular receptor domain further comprises a CD8α signal peptide, which, depending on the embodiment, can comprise the nucleic acid sequence of SEQ ID NO. 4. In several embodiments, the effector domain comprises 4-1BB. In several embodiments, the effector domain comprises a CD16 intracellular domain. In several embodiments, the effector domain comprises 4-1BB and CD16 (with either moiety being “first” vs. “second” in the construct). In several embodiments, repeats of one or more of 4-1BB and/or CD16 are used.

In several embodiments, the chimeric receptor comprises a fragment of NKG2D that is codon optimized and is coupled to a CD8α hinge, a CD3zeta transmembrane region, and an effector domain comprising 4-1BB. In several embodiments, the chimeric receptor is encoded by the nucleic acid sequence of SEQ ID NO: 78. In several embodiments, the chimeric receptor comprises the amino acid sequence of SEQ ID NO: 79.

In several embodiments, the chimeric receptor comprises a fragment of NKG2D that is codon optimized coupled to a CD8α hinge, a CD3zeta transmembrane region, and an effector domain comprising CD16 followed by 4-1BB. In several embodiments, the chimeric receptor comprises the amino acid sequence of SEQ ID NO: 71. In several embodiments, the chimeric receptor is encoded by the nucleic acid sequence of SEQ ID NO: 70.

In several embodiments, the chimeric receptor comprises a fragment of NKG2D that is codon optimized and coupled to a CD8α hinge, a CD3zeta transmembrane region, and an effector domain comprising 4-1BB followed by CD16, optionally coupled by a GS3 linker. In several embodiments, the chimeric receptor comprises the amino acid sequence of SEQ ID NO: 85. In several embodiments, the chimeric receptor is encoded by the nucleic acid sequence of SEQ ID NO: 84.

In several embodiments, the chimeric receptor comprises a fragment of NKG2D that is codon optimized and is coupled to a GS3 linker, an additional NKG2D fragment, a CD8α hinge, a CD3zeta transmembrane region, and an effector domain comprising a CD16 and 4-1BB. In several embodiments, the chimeric receptor is encoded by the nucleic acid sequence of SEQ ID NO: 72. In several embodiments, the chimeric receptor comprises the amino acid sequence of SEQ ID NO: 73.

In several embodiments, the effector domain includes NKp80. In several embodiments, the effector domain is NKp80. In several embodiments, the chimeric receptor comprises a fragment of NKG2D that is coupled to a CD8α hinge, a CD3zeta transmembrane region, and an effector domain comprising a CD16, 4-1BB, and NKp80, and optionally including a GS3 linker. In several embodiments, the chimeric receptor is encoded by the nucleic acid sequence of SEQ ID NO: 74. In several embodiments, the chimeric receptor comprises the amino acid sequence of SEQ ID NO: 75. In several embodiments, the chimeric receptor comprises the fragment of NKG2D that is codon optimized and is coupled to a GS3 linker, an additional NKG2D fragment (optionally codon optimized), a CD8α hinge, a CD3zeta transmembrane region, and an effector domain comprising a CD16, 4-1BB, and NKp80, and optionally including a GS3 linker. In several embodiments, the chimeric receptor is encoded by the nucleic acid sequence of SEQ ID NO: 76. In several embodiments, the chimeric receptor comprises the amino acid sequence of SEQ ID NO: 77. In several embodiments, the chimeric receptor comprises a fragment of NKG2D that is codon optimized and is coupled to a CD8α hinge, a CD3zeta transmembrane region, and an effector domain comprising 4-1BB and NKp80, and optionally including a GS3 linker. In several embodiments, the chimeric receptor is encoded by the nucleic acid sequence of SEQ ID NO: 82. In several embodiments, the chimeric receptor comprises the amino acid sequence of SEQ ID NO: 83.

In several embodiments, the effector domain comprises CD3zeta. In several embodiments, the chimeric receptor comprises a fragment of NKG2D that is codon optimized and is coupled to a CD8α hinge, a CD3zeta transmembrane region, and an effector domain comprising 4-1BB and CD3zeta. In several embodiments, the chimeric receptor is encoded by the nucleic acid sequence of SEQ ID NO: 80. In several embodiments, the chimeric receptor comprises the amino acid sequence of SEQ ID NO: 81.

In several embodiments, the effector domain comprises FcRγ. In several embodiments, the chimeric receptor comprises a fragment of NKG2D coupled to a CD8α hinge, a CD3zeta transmembrane region, and an effector domain comprising 4-1BB and FcRγ. In several embodiments, the chimeric receptor is encoded by the nucleic acid sequence of SEQ ID NO: 86. In several embodiments, the chimeric receptor comprises the amino acid sequence of SEQ ID NO: 87.

In several embodiments, the effector domain comprises CD28. In several embodiments, the chimeric receptor comprises a fragment of NKG2D coupled to a CD8α hinge, a CD3zeta transmembrane region, and an effector domain comprising CD28 and CD3zeta. In several embodiments, the chimeric receptor is encoded by the nucleic acid sequence of SEQ ID NO: 102. In several embodiments, the chimeric receptor comprises the amino acid sequence of SEQ ID NO: 103.

In several embodiments, the effector domain comprises a GS linker.

In several embodiments, the polynucleotides disclosed herein are co-expressed with membrane-bound interleukin 15 (mbIL15).

In several embodiments, a polynucleotide encoding a chimeric receptor comprising an extracellular receptor domain comprising a fragment of NKG2D that is capable of binding a native ligand of NKG2D and is encoded by a fragment of any one of the sequence of SEQ ID NO: 1, of SEQ ID NO. 2, of SEQ ID NO. 3, or SEQ ID NO. 68, and an effector domain comprising a transmembrane region and an intracellular signaling domain. In several embodiments, there is provided a polynucleotide encoding a chimeric receptor comprising an extracellular receptor domain comprising a fragment of NKG2D that is capable of binding a native ligand of NKG2D and is encoded by (i) a fragment of the sequence of SEQ ID NO: 1, (ii) the sequence of SEQ ID NO. 2, (iii) the sequence of SEQ ID NO. 3, or (iv) the sequence of SEQ ID NO. 68, and an effector domain comprising a transmembrane region and an intracellular signaling domain. In several embodiments, a polynucleotide encoding a chimeric receptor comprising an extracellular receptor domain comprising a fragment of NKG2D that is capable of binding a native ligand of NKG2D and is encoded by the sequence of SEQ ID NO. 2, and an effector domain comprising a transmembrane region and an intracellular signaling domain. In several embodiments, a polynucleotide encoding a chimeric receptor comprising an extracellular receptor domain comprising a fragment of NKG2D that is capable of binding a native ligand of NKG2D and is encoded by the sequence of SEQ ID NO. 3, and an effector domain comprising a transmembrane region and an intracellular signaling domain. In several embodiments, a polynucleotide encoding a chimeric receptor comprising an extracellular receptor domain comprising a fragment of NKG2D that is capable of binding a native ligand of NKG2D and is encoded by a fragment of the sequence of SEQ ID NO. 68, and an effector domain comprising a transmembrane region and an intracellular signaling domain. In several embodiments, the extracellular receptor domain comprises a hinge region. In several embodiments, the hinge region is a CD8α hinge encoded by the nucleic acid sequence of SEQ ID NO: 5, or optionally a fragment of the nucleic acid sequence of SEQ ID NO: 5 (e.g., a fragment having about 75%, about 85%, about 95% homology to SEQ ID NO: 5). In several embodiments, the hinge region is an Immunoglobulin G4 (IgG4) hinge encoded by the nucleic acid sequence of SEQ ID NO: 104. In several embodiments, the hinge region is an Immunoglobulin G4 (IgG4) hinge encoded by a fragment of the nucleic acid sequence of SEQ ID NO: 104 (e.g., a fragment having about 75%, about 85%, about 95% homology to SEQ ID NO: 104). In several embodiments, the extracellular receptor domain further comprises a CD8α signal peptide, wherein the signal peptide comprises the nucleic acid sequence of SEQ ID NO. 4. In several embodiments, the effector domain comprises at least one signaling domains selected from the group consisting of OX40 (CD134), CD3zeta, 4-1BB, CD28 and DAP12. In several embodiments, the chimeric receptor transmembrane domain comprises a CD8 transmembrane domain. In several embodiments, the chimeric receptor comprises IL-15 linked (optionally by a GS3 linker) to the fragment of NKG2D coupled to a CD8α hinge, a CD8α transmembrane domain, 4-1BB, and CD3zeta. In several embodiments, the chimeric receptor is encoded by the nucleic acid sequence of SEQ ID NO: 88. In several embodiments, the chimeric receptor comprises the amino acid sequence of SEQ ID NO: 89.

In several embodiments, the chimeric receptor comprises a fragment of NKG2D coupled to an IgG4 hinge, a CD8α transmembrane domain, 4-1BB, and CD3zeta. In several embodiments, the chimeric receptor is encoded by the nucleic acid sequence of SEQ ID NO: 96. In several embodiments, the chimeric receptor comprises the amino acid sequence of SEQ ID NO: 97.

In several embodiments, the effector domain comprises OX40. In several embodiments, the chimeric receptor comprises the fragment of NKG2D coupled to a CD8α hinge, a CD8α transmembrane domain, OX40, and CD3zeta. In several embodiments, the chimeric receptor is encoded by the nucleic acid sequence of SEQ ID NO: 90. In several embodiments, the chimeric receptor is encoded by the nucleic acid sequence of SEQ ID NO: 109. In several embodiments, the chimeric receptor comprises the amino acid sequence of SEQ ID NO: 91. In several embodiments, the chimeric receptor comprises a fragment of NKG2D coupled to an IgG4 hinge, a CD8α transmembrane domain, OX40 and CD3zeta. In several embodiments, the chimeric receptor is encoded by the nucleic acid sequence of SEQ ID NO: 100. In several embodiments, the chimeric receptor comprises the amino acid sequence of SEQ ID NO: 101.

In several embodiments, the chimeric receptor comprises a CD28 transmembrane/intracellular domain. In several embodiments, the chimeric receptor comprises the fragment of NKG2D coupled to a CD8α hinge, a CD28 transmembrane/intracellular domain, and CD3zeta. In several embodiments, the chimeric receptor is encoded by the nucleic acid sequence of SEQ ID NO: 92. In several embodiments, the chimeric receptor comprises the amino acid sequence of SEQ ID NO: 93.

In several embodiments, the chimeric receptor comprises a fragment of NKG2D coupled to a CD8α hinge, a CD28 transmembrane/intracellular domain, 4-1BB, and CD3zeta. In several embodiments, the chimeric receptor is encoded by the nucleic acid sequence of SEQ ID NO: 94. In several embodiments, the chimeric receptor comprises the amino acid sequence of SEQ ID NO: 95.

In several embodiments, the chimeric receptor comprises a fragment of NKG2D coupled to an IgG4 hinge, a CD28 transmembrane/intracellular domain and CD3zeta. In several embodiments, the chimeric receptor is encoded by the nucleic acid sequence of SEQ ID NO: 98. In several embodiments, the chimeric receptor comprises the amino acid sequence of SEQ ID NO: 99.

In several embodiments, the effector domain comprises a GS linker. In several embodiments, the polynucleotides disclosed herein are configured to be co-expressed (either on the same polynucleotide, or another polynucleotide) with membrane-bound interleukin 15 (mbIL15).

Any of the chimeric receptors can optionally include an extracellular receptor domain that includes a second peptide that binds native ligands of NKG2D. In several embodiments, the second peptide is homologous with NKG2D, while in other embodiments, the second peptide is heterologous with respect to the NKG2D. Whether the chimeric receptor includes a dimerized extracellular receptor domain, the extracellular receptor domains can recognize at least the following native ligands of NKG2D: MICA, MICB, ULBP1, ULBP2, ULBP3, ULBP4, ULBP5 or ULBP6.

As discussed in more detail below, functional variants of the NKG2D ligand binding domains are employed in several embodiments. For example the peptide that binds native ligands of NKG2D has, in several embodiments, at least 80% homology to SEQ ID NO: 1, SEQ ID NO: 2, SEQ ID NO: 3, or SEQ ID NO: 68. In several embodiments, the peptide that binds native ligands of NKG2D has at least 70%, at least 75%, at least 80%, at least 85%, at least 90%, or at least 95% homology to SEQ ID NO: 1, SEQ ID NO: 2, SEQ ID NO: 3, or SEQ ID NO: 68.

Additionally provided for herein in several embodiments are vectors for expressing the chimeric receptors. In several embodiments, the polynucleotides provided for herein are mRNA and can include an operable linkage to least one regulatory element for the expression of the chimeric receptor. In several embodiments, the polynucleotides further include one or more internal ribosome entry site (IRES). In several embodiments, the vector is a retrovirus.

Engineered natural killer cells are also provided for, in several embodiments, that express any of the chimeric receptor constructs disclosed herein, the engineered NK cells exhibiting enhanced cytotoxic effects against target cells. Enhanced cytotoxic effects include, but are not limited to, higher affinity for target (e.g., cancerous) cells as compared to normal (e.g., non-cancerous) cells, a greater killing effect directed against target cells, reduced off-target effects, increased duration of cytotoxic effects, more efficient cytotoxicity, and the like. Such enhanced effects can be identified through the use of various in vitro cytotoxicity assays (e.g., measurement of cytokine production, etc.), measurement of target cell death, or through various clinical outcomes (e.g., reduction in tumor burden). In several embodiments, the engineered NK cells are an autologous cell isolated from a patient. In additional embodiments, the engineered NK cells are generated from allogeneic cells isolated from a donor. Such engineered NK cells as disclosed herein are used, in several embodiments, to enhance NK cell cytotoxicity in a mammal in need thereof, by administering the NK cells. These engineered NK cells are used, in several embodiments for treating or preventing cancer or an infectious disease in a mammal. The polynucleotides encoding, the vectors carrying, and the NK cells expressing the various chimeric receptors disclosed herein can also be used, in several embodiments in the manufacture of a medicament for enhancing NK cell cytotoxicity (e.g., to treat or prevent cancer or an infectious disease). In several embodiments, the chimeric receptor constructs disclosed herein do not significantly increase the cytotoxicity of the engineered NK cells against normal cells and, as described herein, are advantageously improved as compared to non-engineered NK cells. In several embodiments, there is provided a polynucleotide encoding a chimeric receptor comprising an extracellular receptor domain, a transmembrane region, and an effector domain. In several embodiments, the extracellular receptor domain comprises a peptide that binds native ligands of Natural Killer Group 2 member D (NKG2D), wherein the peptide that binds native ligands of NKG2D is a fragment of NKG2D. Several embodiments, relate to a polynucleotide encoding a chimeric receptor comprising: (a) an extracellular receptor domain, wherein said extracellular receptor domain comprises a peptide that binds native ligands of Natural Killer Group 2 member D (NKG2D), wherein the peptide that binds native ligands of NKG2D is a fragment of NKG2D, wherein the fragment of NKG2D is encoded by a polynucleotide comprising: (i) a fragment of the sequence of SEQ ID NO: 1, (ii) the sequence of SEQ ID NO. 2, (iii) the sequence of SEQ ID NO. 3, or (iv) the sequence of SEQ ID NO. 68, (b) a transmembrane region, and (c) an effector domain.

In several embodiments, there is provided a polynucleotide encoding a chimeric receptor comprising: (a) an extracellular receptor domain, wherein said extracellular receptor domain comprises a peptide that binds native ligands of Natural Killer Group 2 member D (NKG2D), wherein the peptide that binds native ligands of NKG2D is a fragment of NKG2D, wherein the fragment of NKG2D is encoded by a polynucleotide comprising: (i) a fragment of the sequence of SEQ ID NO: 1, (ii) the sequence of SEQ ID NO. 2, (iii) the sequence of SEQ ID NO. 3, (iv) or the sequence of SEQ ID NO. 68; and (b) an effector domain comprising a transmembrane region and an intracellular signaling domain.

In several embodiments, the transmembrane region comprises a CD3zeta transmembrane region. In several embodiments, the CD3zeta transmembrane region comprises the amino acid sequence of SEQ ID NO: 69. In several embodiments, the transmembrane region comprises CD8α. In several embodiments, the effector domain comprises 4-1BB, an intracellular domain of 2B4, NKp80, a CD16 intracellular domain, Natural Cytotoxicity Triggering Receptor 1 (NCR1), Natural Cytotoxicity Triggering Receptor 2 (NCR2), Natural Cytotoxicity Triggering Receptor 3 (NCR3), and/or an intracellular domain of DAP10. In one embodiment, the effector domain comprises 4-1BB and CD16. In several embodiments, the effector domain comprises 4-1BB and CD3 zeta. In several embodiments, the effector domain comprises 4-1BB and an intracellular domain of 2B4 or DAP10. In several embodiments, the effector domain comprises 2B4 followed by 4-1BB while in other embodiments the effector domain comprises 4-1BB followed by 2B4. In several embodiments, the effector domain comprises DAP10 followed by 4-1BB. In several embodiments, the effector domain comprises 4-1BB followed by DAP10. In several embodiments, the effector domain further comprises CD3zeta. In several embodiments, the effector domain comprises at least one signaling domain selected from the group consisting of OX40 (CD134), CD3zeta, 4-1BB, CD28 and DAP12. In several embodiments the effector domain comprises one or more hemi-ITAM sequences. In several embodiments, the hemi-ITAM comprises the amino acid sequence of SEQ ID NO. 14. In several embodiments, the hemi-ITAM comprises the amino acid sequence of SEQ ID NO. 37. In several embodiments, the effector domain comprises one or more ITSM sequences. In several embodiments, the ITSM comprises the amino acid sequence of SEQ ID NO. 15 or the amino acid sequence of SEQ ID NO. 35

In several embodiments, the chimeric receptor comprises a fragment of NKG2D coupled to a CD8α hinge, a CD8α transmembrane domain, 4-1BB, 2B4, and CD3zeta. In one embodiment, the chimeric receptor is encoded by the nucleic acid sequence of SEQ ID NO: 58. In one embodiment, the chimeric receptor comprises the amino acid sequence of SEQ ID NO: 59. In several embodiments, the chimeric receptor comprises a fragment of NKG2D coupled to a CD8α hinge, a CD8α transmembrane domain, 4-1BB, and DAP10. In several embodiments, the chimeric receptor is encoded by the nucleic acid sequence of SEQ ID NO: 60 and comprises the amino acid sequence of SEQ ID NO: 61.

In several embodiments, the chimeric receptor comprises a fragment of NKG2D coupled to a CD8α hinge, a CD8α transmembrane domain, 4-1BB, 2B4, and DAP10. In several embodiments, the effector domain comprises 4-1BB, followed by DAP10, followed by 2B4. In some embodiments, the chimeric receptor is encoded by the nucleic acid sequence of SEQ ID NO: 62 and the chimeric receptor comprises the amino acid sequence of SEQ ID NO: 63. In several embodiments, the effector domain comprises 4-1BB, followed by 2B4, followed by DAP10. In several embodiments, the chimeric receptor is encoded by the nucleic acid sequence of SEQ ID NO: 64 and the chimeric receptor comprises the amino acid sequence of SEQ ID NO: 65.

In several embodiments, the chimeric receptor comprises a fragment of NKG2D that is codon optimized coupled to a GS3 linker, an additional NKG2D fragment, a CD8α hinge, a CD8α transmembrane domain, 4-1BB, and CD3zeta. In one embodiment, the chimeric receptor is encoded by the nucleic acid sequence of SEQ ID NO: 66. In several embodiments, the chimeric receptor comprises the amino acid sequence of SEQ ID NO: 67.

In several embodiments, the chimeric receptor comprises a fragment of NKG2D that is codon optimized coupled to a CD8α hinge, a CD3zeta transmembrane region, and an effector domain comprising 4-1BB, is encoded by the nucleic acid sequence of SEQ ID NO: 78 and/or comprises the amino acid sequence of SEQ ID NO: 79.

In several embodiments, the chimeric receptor comprises a fragment of NKG2D that is codon optimized coupled to a CD8α hinge, a CD3zeta transmembrane region, and an effector domain comprising CD16 followed by 4-1BB. In several embodiments, the chimeric receptor is encoded by the nucleic acid sequence of SEQ ID NO: 70 and/or comprises the amino acid sequence of SEQ ID NO: 71.

In several embodiments, the chimeric receptor comprises a fragment of NKG2D that is codon optimized coupled to a CD8α hinge, a CD3zeta transmembrane region, and an effector domain comprising 4-1BB followed by a GS3 linker and CD16. In one embodiment, the chimeric receptor comprises the amino acid sequence of SEQ ID NO: 85 and/or is encoded by the nucleic acid sequence of SEQ ID NO: 84.

In several embodiments, the chimeric receptor comprises a fragment of NKG2D that is codon optimized coupled to a GS3 linker, an additional NKG2D fragment, a CD8α hinge, a CD3zeta transmembrane region, and an effector domain comprising a CD16 and 4-1BB. In one embodiment, the chimeric receptor is encoded by the nucleic acid sequence of SEQ ID NO: 72 and/or comprises the amino acid sequence of SEQ ID NO: 73.

In several embodiments, the chimeric receptor comprises IL-15 linked by a GS3 linker to the fragment of NKG2D coupled to a CD8α hinge, a CD8α transmembrane domain, 4-1BB, and CD3zeta, is encoded by the nucleic acid sequence of SEQ ID NO: 88 and/or comprises the amino acid sequence of SEQ ID NO: 89.

In several embodiments, the chimeric receptor comprises the fragment of NKG2D coupled to a IgG4 hinge, a CD8α transmembrane domain, 4-1BB, and CD3zeta is encoded by the nucleic acid sequence of SEQ ID NO: 96, and/or comprises the amino acid sequence of SEQ ID NO: 97.

In several embodiments, the chimeric receptor comprises the fragment of NKG2D coupled to a CD8α hinge, a CD8α transmembrane domain, OX40, and CD3zeta, is encoded by the nucleic acid sequence of SEQ ID NO: 90, and/or comprises the amino acid sequence of SEQ ID NO: 91.

In several embodiments, the chimeric receptor comprises the fragment of NKG2D coupled to an IgG4 hinge, a CD8α transmembrane domain, OX40 and CD3zeta, is encoded by the nucleic acid sequence of SEQ ID NO: 100, and/or comprises the amino acid sequence of SEQ ID NO: 101.

In several embodiments, the chimeric receptor comprises the fragment of NKG2D coupled to a CD8α hinge, a CD28 transmembrane/intracellular domain, and CD3zeta, is encoded by the nucleic acid sequence of SEQ ID NO: 92, and/or comprises the amino acid sequence of SEQ ID NO: 93.

In several embodiments, the chimeric receptor comprises the fragment of NKG2D coupled to a CD8α hinge, a CD28 transmembrane/intracellular domain, 4-1BB, and CD3zeta, is encoded by the nucleic acid sequence of SEQ ID NO: 94, and/or comprises the amino acid sequence of SEQ ID NO: 95.

In several embodiments, the chimeric receptor comprises the fragment of NKG2D coupled to an IgG4 hinge, a CD28 transmembrane/intracellular domain and CD3zeta, is encoded by the nucleic acid sequence of SEQ ID NO: 98, and/or comprises the amino acid sequence of SEQ ID NO: 99.

In several embodiments, the chimeric receptor comprises a fragment of NKG2D coupled to a CD8α hinge, a CD3zeta transmembrane region, and an effector domain comprising a CD16, 4-1BB, a GS3 linker, and NKp80. In one embodiment, the chimeric receptor is encoded by the nucleic acid sequence of SEQ ID NO: 74 and/or comprises the amino acid sequence of SEQ ID NO: 75.

In several embodiments, the chimeric receptor comprises the fragment of NKG2D that is codon optimized coupled to a GS3 linker, an additional NKG2D fragment, a CD8α hinge, a CD3zeta transmembrane region, and an effector domain comprising a CD16, 4-1BB, a GS3 linker, and NKp80. In one embodiment, the chimeric receptor is encoded by the nucleic acid sequence of SEQ ID NO: 76 and/or comprises the amino acid sequence of SEQ ID NO: 77. In several embodiments, the chimeric receptor comprises a fragment of NKG2D that is codon optimized coupled to a CD8α hinge, a CD3zeta transmembrane region, and an effector domain comprising 4-1BB, a GS3 linker, and NKp80. In one embodiment, the chimeric receptor is encoded by the nucleic acid sequence of SEQ ID NO: 82 and/or comprises the amino acid sequence of SEQ ID NO: 83.

In several embodiments, the chimeric receptor comprises a fragment of NKG2D that is codon optimized coupled to a CD8α hinge, a CD3zeta transmembrane region, and an effector domain comprising 4-1BB and CD3zeta. In one embodiment, the chimeric receptor is encoded by the nucleic acid sequence of SEQ ID NO: 80 and/or comprises the amino acid sequence of SEQ ID NO: 81.

Depending on the embodiment, the effector domain may also comprise FcRγ. For example, in several embodiments, the chimeric receptor comprises a fragment of NKG2D coupled to a CD8α hinge, a CD3zeta transmembrane region, and an effector domain comprising 4-1BB and FcRγ. In one embodiment, the chimeric receptor is encoded by the nucleic acid sequence of SEQ ID NO: 86 and/or comprises the amino acid sequence of SEQ ID NO: 87.

Depending on the embodiment, the effector domain may also comprise CD28. For example, in several embodiments, the chimeric receptor comprises a fragment of NKG2D coupled to a CD8α hinge, a CD3zeta transmembrane region, and an effector domain comprising CD28 and CD3zeta. In several embodiments, the chimeric receptor is encoded by the nucleic acid sequence of SEQ ID NO: 102 and/or comprises the amino acid sequence of SEQ ID NO: 103.

In several embodiments, the effector domain comprises a GS linker.

In several embodiments, the extracellular receptor domain further comprises a CD8α signal peptide, wherein the signal peptide comprises the nucleic acid sequence of SEQ ID NO. 4. In several embodiments, the extracellular receptor domain further comprises 2 extracellular residues of CD3zeta directly adjacent to the CD3zeta transmembrane region. In several embodiments, the extracellular receptor domain comprises a CD8α signal peptide, wherein the signal peptide comprises the nucleic acid sequence of SEQ ID NO. 4.

In several embodiments, the chimeric receptor comprises one or more GS3 linkers. In several embodiments, the chimeric receptor domain comprises a hinge region. In several embodiments, the hinge region is encoded by the nucleic acid sequence of SEQ ID NO: 5, while in some embodiments, the hinge region is encoded by a fragment of the nucleic acid sequence of SEQ ID NO: 5. In several embodiments, the hinge region is a CD8α hinge. In several embodiments, the hinge region comprises a glycine-serine repeating motif having the amino acid sequence of SEQ ID NO: 31. In several embodiments, the hinge region comprises the amino acid sequence of SEQ ID NO: 32 and in some embodiments, the hinge region comprises the amino acid sequence of SEQ ID NO: 33. In additional embodiments, the hinge region is encoded by the nucleic acid sequence of SEQ ID NO: 34. In several embodiments, the hinge region comprises a portion of the beta-adrenergic receptor. In some such embodiments, the hinge region is encoded by the nucleic acid sequence of SEQ ID NO: 40. In additional embodiments, the hinge region is encoded by the nucleic acid sequence of SEQ ID NO: 42. In several embodiments, the hinge region is Immunoglobulin G4 (IgG4) hinge encoded by the nucleic acid sequence of SEQ ID NO: 104. In several embodiments, the hinge region is an Immunoglobulin G4 (IgG4) hinge encoded by a fragment of the nucleic acid sequence of SEQ ID NO: 104. In several embodiments, the chimeric receptor comprises the fragment of NKG2D coupled to a CD8α hinge and a CD8α transmembrane domain.

In one embodiment, the chimeric receptor comprises the fragment of NKG2D coupled to CD16, is encoded by the nucleic acid sequence of SEQ ID NO: 23, and/or comprises the amino acid sequence of SEQ ID NO: 24. In one embodiment, the chimeric receptor comprises the fragment of NKG2D coupled to NCR1. In some such embodiments, the chimeric receptor is encoded by the nucleic acid sequence of SEQ ID NO: 27 and/or comprises the amino acid sequence of SEQ ID NO: 28. In several embodiments, the chimeric receptor comprises at least a portion of the amino acid sequence of SEQ ID NO: 21. In several embodiments, the chimeric receptor comprises the fragment of NKG2D coupled to NCR3, in several embodiments is encoded by the nucleic acid sequence of SEQ ID NO. 29 and/or comprises the amino acid sequence of SEQ ID NO. 30.

In several embodiments, the chimeric receptor comprises the fragment of NKG2D coupled to a CD16 transmembrane/intracellular domain and 4-1BB. In several embodiments, the chimeric receptor comprises the fragment of NKG2D coupled to a CD8α hinge, a CD16 transmembrane/intracellular domain and 4-1BB, is encoded by the nucleic acid sequence of SEQ ID NO: 25, and/or comprises the amino acid sequence of SEQ ID NO: 26.

In several embodiments, the chimeric receptor comprises the fragment of NKG2D coupled to NCR1 and 4-1BB, wherein the chimeric receptor comprises the NCR1 amino acid sequence of SEQ ID NO: 20.

In several embodiments, the chimeric receptor comprises the fragment of NKG2D coupled to CD8α, 4-1BB and CD3zeta, is encoded by the nucleic acid sequence of SEQ ID NO. 18 and/or comprises the amino acid sequence of SEQ ID NO. 19.

In several embodiments, the chimeric receptor comprises the fragment of NKG2D coupled to NCR3 and 4-1BB, and wherein the NCR3 comprises the amino acid sequence of SEQ ID NO: 22. In one embodiment, the chimeric receptor comprises one or more of the NCR1 transmembrane/intracellular domain of SEQ ID NO: 20 or the NCR3 transmembrane/intracellular domain of SEQ ID NO: 22.

In several embodiments, the chimeric receptor comprises the fragment of NKG2D coupled to a GS3 linker, a CD8α hinge, a CD16 transmembrane/intracellular domain and 4-1BB. In several embodiments, the chimeric receptor is encoded by the nucleic acid sequence of SEQ ID NO: 43. In several embodiments, the chimeric receptors comprises the fragment of NKG2D coupled to a GS3 linker, a CD16 transmembrane/intracellular domain and 4-1BB. In one embodiment, the chimeric receptor is encoded by the nucleic acid sequence of SEQ ID NO: 44.

In several embodiments, the chimeric receptor comprises the fragment of NKG2D coupled to a CD16 transmembrane/intracellular domain and 4-1BB and is encoded by the nucleic acid sequence of SEQ ID NO: 45.

In several embodiments, the chimeric receptor comprises the fragment of NKG2D coupled to a CD8α hinge, a CD8α transmembrane domain, 4-1BB, and 2B4 and is encoded by the nucleic acid sequence of SEQ ID NO: 46.

In several embodiments, the chimeric receptor comprises the fragment of NKG2D coupled to a beta-adrenergic extracellular domain, a beta-adrenergic transmembrane domain, 4-1BB, and 2B4 and is encoded by the nucleic acid sequence of SEQ ID NO: 47.

In several embodiments the chimeric receptor comprises the fragment of NKG2D coupled to a CD8α hinge, a CD8α transmembrane domain, 4-1BB, 2B4, a GS3 linker, and NKp80 and is encoded by the nucleic acid sequence of SEQ ID NO: 48.

In several embodiments, the chimeric receptor comprises the fragment of NKG2D coupled to a CD8α hinge, a CD8α transmembrane domain, 4-1BB, a GS3 linker, and NKp80 and is encoded by the nucleic acid sequence of SEQ ID NO: 49.

In several embodiments, the chimeric receptor comprises the fragment of NKG2D that is codon optimized coupled to a GS3 linker, an additional NKG2D fragment, a beta-adrenergic extracellular domain, a beta-adrenergic transmembrane domain, 4-1BB, an additional GS3 linker, and NKp80 and is encoded by the nucleic acid sequence of SEQ ID NO: 50.

In several embodiments, the chimeric receptor comprises the fragment of NKG2D that is codon optimized coupled to a GS3 linker, an additional NKG2D fragment, a CD8α hinge, a CD8α transmembrane domain, 4-1BB, an additional GS3 linker, and NKp80 and is encoded by the nucleic acid sequence of SEQ ID NO: 51.

In several embodiments, the chimeric receptor comprises the fragment of NKG2D that is codon optimized coupled to a GS3 linker, an additional NKG2D fragment, a CD8α hinge, a CD16 transmembrane/intracellular domain, and 4-1BB and is encoded by the nucleic acid sequence of SEQ ID NO: 52.

In several embodiments, the chimeric receptor comprises the fragment of NKG2D coupled to a CD8α hinge, a CD16 transmembrane/intracellular domain, 4-1BB, and 2B4 and is encoded by the nucleic acid sequence of SEQ ID NO: 53.

In several embodiments, the chimeric receptor comprises the fragment of NKG2D coupled to a CD8α hinge, a CD16 transmembrane/intracellular domain, 4-1BB, a GS3 linker, and NKp80 and is encoded by the nucleic acid sequence of SEQ ID NO: 54.

In several embodiments, the chimeric receptor constructs are encoded by a polynucleotide that encodes a chimeric receptor wherein the extracellular receptor domain comprises a second peptide that binds native ligands of NKG2D, (e.g., one or more of MICA, MICB, ULBP1, ULBP2, ULBP3, ULBP4, ULBP5 or ULBP6. Depending on the embodiment, the peptide that binds native ligands of NKG2D has at least 80% homology to SEQ ID NO: 1, SEQ ID NO: 2, or SEQ ID NO. 3.

In several embodiments, the polynucleotide is co-expressed with an additional construct encoding membrane-bound interleukin 15 (mbIL15). In several embodiments, the chimeric receptor is encoded by the nucleic acid sequence of SEQ ID NO: 18. In several embodiments, the chimeric receptor is encoded by the amino acid sequence of SEQ ID NO: 19.

According to several embodiments, the chimeric receptor does not comprise DNAX-activating protein 10 (DAP10) and/or the chimeric receptor does not encode an immunoreceptor tyrosine-based activation (ITAM) motif.

In several embodiments, the polynucleotides disclosed herein are mRNA. Additionally, in several embodiments, the polynucleotide disclosed herein are operably linked to at least one regulatory element for the expression of the chimeric receptor.

Also provided for herein are vectors that comprise the polynucleotides disclosed herein. In several embodiments, the polynucleotide is operatively linked to at least one regulatory element for expression of the chimeric receptor. In several embodiments, the vector is a retrovirus.

Also provided for herein are genetically engineered natural killer cells comprising the any one or more of the polynucleotides disclosed herein. In several embodiments, the natural killer cells are for autologous use, while in some embodiments they are for allogeneic use.

Also provided for herein are methods of enhancing NK cell cytotoxicity in a mammal in need thereof, comprising administering to the mammal NK cells, wherein said NK cells express a chimeric receptor encoded by a polynucleotide disclosed herein.

Additionally, there are provided methods for treating or preventing cancer or an infectious disease in a mammal in need thereof, said method comprising administering to said mammal a therapeutically effective amount of NK cells, wherein said NK cells express a chimeric receptor encoded by a polynucleotide disclosed herein. As disclosed above, the NK cells can be allogeneic or autologous.

There is provided a use of a polynucleotide as disclosed herein in the manufacture of a medicament for enhancing NK cell cytotoxicity in a mammal in need thereof. Further there is provided a use of a polynucleotide in the manufacture of a medicament for treating or preventing cancer or an infectious disease in a mammal in need thereof.

Also provided is the use of a vector comprising a polynucleotide disclosed herein in the manufacture of a medicament for enhancing NK cell cytotoxicity in a mammal in need thereof. Also provided is the use of a vector comprising a polynucleotide disclosed herein in the manufacture of a medicament for treating or preventing cancer or an infectious disease in a mammal in need thereof.

Also provided is the use of an isolated genetically engineered natural killer cell expressing a chimeric receptor as disclosed herein for enhancing NK cell cytotoxicity in a mammal in need thereof. Also provided is the use of an isolated genetically engineered natural killer cell expressing a chimeric receptor as disclosed herein for treating or preventing cancer or an infectious disease in a mammal in need thereof.

The compositions and related methods summarized above and set forth in further detail below describe certain actions taken by a practitioner; however, it should be understood that they can also include the instruction of those actions by another party. Thus, actions such as “administering a population of NK cells expressing a chimeric receptor” include “instructing the administration of a population of NK cells expressing a chimeric receptor.”

### BRIEF DESCRIPTION OF THE DRAWINGS

The descriptions of the figures below are related to experiments and results that represent non-limiting embodiments of the inventions disclosed herein.

FIGS. 1A-1C depict schematic representations of the chimeric receptors according to several embodiments disclosed herein. FIG. 1A depicts endogenous NKG2D, FIG. 1B depicts NKG2D-DAP10-CD3zeta, and FIG. 1C depicts NKG2D-41BB-CD3zeta.

FIGS. 2A-2B depict schematic representations of the chimeric receptors, according to several embodiments disclosed herein. FIG. 2A depicts NKG2DCD16 and FIG. 2B depicts NKG2D-CD16-41BB.

FIGS. 3A-3B depict plasmid maps illustrating the point of insertion of certain constructs according to several embodiments into the plasmids, illustrated is a Murine Stem Cell Virus (MSCV) plasmid. FIG. 3A shows gene constructs for NKG2D-DAP10-CD3zeta and NKG2D-41BB-CD3zeta that were inserted into the EcoRI and NotI restriction sites, with removal the IRES-GFP sequence in the vector. FIG. 3B depicts the plasmids for NKG2D-CD16 and NKG2D-CD16-41BB that were inserted into EcoRI and XhoI restriction sites located in the multiple cloning site (MCS). IRES-GFP sequence in the vector allows for the tracing of transduction efficiency.

FIGS. 4A-4C depict data related to the expression of NKG2D-DAP10-CD3zeta and NKG2D-41BB-CD3zeta in NK cells. FIG. 4A shows flow cytometry data illustrating the percentage of NKG2D-positive NK cells after transduction. FIG. 4B shows a dot plots summarizing the percentage of NKG2D-positive NK cells. FIG. 4C shows data related to the mean fluorescence intensity (MFI) in different group of NK cells after transduction.

FIGS. 5A-5C depict data related to the cytotoxicity of the various constructs generated from NK cells from Donor 1, Donor 2, and Donor 3 (FIGS. 5A, 5B, and 5C, respectively) against cultured REH cells.

FIGS. 6A-6C depict data related to the cytotoxicity of the various constructs generated from NK cells from Donor 1, Donor 2, and Donor 3 (FIGS. 6A, 6B, and 6C, respectively) against cultured U-2 OS cells.

FIGS. 7A-7B depict data related to the production of interferon-gamma by NK cells expressing various NKG2D constructs in the presence and absence of stimulation with REH cells. FIG. 7A depicts the relative amount of IFNγ in the different groups of NK cells with or without stimulation by REH cells. FIG. 7B depicts levels of IFNγ between different groups of NK cells after stimulation (median values represented).

FIGS. 8A-8C depict data related to the expression of NKG2D-DAP10-CD3zeta and NKG2D-CD16 in NK cells. FIG. 8A shows flow cytometry data illustrating the percentage of NKG2D-positive NK cells after transduction. FIG. 8B shows a dot plots summarizing the percentage of NKG2D-positive NK cells. FIG. 8C shows data related to the mean fluorescence intensity (MFI) in different group of NK cells after transduction.

FIGS. 9A-9C depict data related to the cytotoxicity of the various constructs generated from NK cells from 3 donors (FIGS. 9A, 9B, and 9C, respectively) against cultured REH cells.

FIGS. 10A-10C depict data related to the cytotoxicity of the various constructs generated from NK cells from 3 donors (FIGS. 10A, 10B, and 10C, respectively) against cultured U-2 OS cells.

FIG. 11 depicts data related to the production of interferon-gamma by NK cells expressing various NKG2D constructs in the presence and absence of stimulation with REH cells.

FIGS. 12A-12B depict data related to expression of NKG2D-DAP10-CD3zeta and NKG2D-CD16-41BB in NK cells. FIG. 12A shows flow cytometry data illustrating the percentage of NKG2D-positive NK cells after transduction. FIG. 12B shows a histogram related to relative amount of surface expression of the various constructs on NK cells.

FIGS. 13A-13B depict data related to the degree of cytotoxicity of various NKG2d constructs. FIG. 13A depicts the degree of cytotoxicity against cultured REH cells.

FIG. 13B depicts the degree of cytotoxicity against cultured U2OS cells.

FIG. 14 schematically depicts construct maps of several NKG2D constructs according to some embodiments disclosed herein.

FIG. 15 schematically depicts construct maps of additional NKG2D constructs according to some embodiments disclosed herein.

FIGS. 16A-16C depict data related to the expression of the various NKG2D constructs in NK cells. FIG. 16A shows data related to the mean fluorescence intensity (MFI) of the various NKG2D constructs in NK cells. FIG. 16B shows flow cytometry data illustrating the percentage of NKG2D-positive and CD56-positive NK cells after transduction of various NKG2D constructs into the NK cells of two donors (505 and 870). FIG. 16C shows data related to the mean fluorescence intensity (MFI) in NK cells from 2 donors seven days after transduction.

FIG. 17 depicts data related to the cytotoxicity of the various NKG2D constructs 14 days post-transduction into NK cells at a 1:1 E:T ratio.

FIGS. 18A-18B depicts data related to the expression of the various NKG2D constructs following transduction into NK cells. FIG. 18A shows data related to the mean fluorescence intensity (MFI) in NK cells seven days after transduction. FIG. 18B shows data related to the fold-change in MFI of the various NKG2D constructs relative to the mock-transduced NK cells.

FIGS. 19A-19B depict data related to the cytotoxicity of the various NKG2D constructs. FIG. 19A shows data related to the cytotoxicity of the various NKG2D constructs transduced into NK cells at a 1:1 E:T ratio. FIG. 19B shows data related to the percent change in cytotoxicity of the various NKG2D constructs relative to the mock-transduced NK cells.

FIG. 20 depicts data related to the cytotoxicity of the various NKG2D constructs 14 days post-transduction into NK cells at a 1:1 E:T ratio. Prior to analysis NK cells were cultured in media supplemented with 40 IU of IL-2/mL.

FIG. 21 depicts data related to the cytotoxicity of the various NKG2D constructs 10 days post-transduction into Donor 238 NK cells (with 4 days of culturing in media supplemented with 40 IU of IL-2/mL every two days) against cultured REH cells at 1:1 and 1:2 E:T ratios for two hours.

FIG. 22 schematically depicts construct maps of additional NKG2D constructs according to embodiments disclosed herein.

FIGS. 23A-23B depict data related to the persistence of the various NKG2D constructs generated from NK cells from two different donors (Donor 61 and Donor 103 in FIGS. 23A and 23B, respectively). NK cells were cultured in media supplemented with 40 IU of IL-2/mL.

FIG. 24 depicts data related to the expression of the various NKG2D constructs. NK cells were expanded from peripheral blood mononuclear cells (PBMC) of 4 healthy donors (224, 225, 362 and 363) and transduced with viruses directing the expression of the indicated constructs. Three days following transduction, NK cells were stained with a fluorescently labelled anti-NKG2D antibody and analyzed using flow cytometry. Relative NKG2D expression was assessed by mean fluorescence intensity (MFI) of labeled cells.

FIGS. 25A-25B depict data related to the cytotoxicity of NK cells transduced with various NKG2D constructs. NK cells were expanded from PBMC of 4 donors; Eight days after transduction, NK cytotoxicity against cultured REH and HL60 cells (FIGS. 25A and 25B, respectively) was measured at a 1:1 E:T ratio. NK cells were cultured in media supplemented with 40 IU of IL-2/mL prior to analysis.

FIGS. 26A-26C depict data related to the production of interferon-gamma (IFNγ), tumor necrosis factor-alpha (TNFα), and granulocyte-macrophage colony-stimulating factor (GM-CSF) by NK cells expressing various NKG2D constructs after overnight stimulation with REH tumor cells. Eight days after transduction with the indicated constructs, 1×10⁵ NK cells were stimulated with 1×10⁵ REH cells in individual wells of a 96-well round bottom plate; after overnight incubation, supernatants were harvested, and cytokine levels measured against relevant standards using a Meso Scale Discovery device. FIG. 26A depicts the accumulated levels of IFNγ, FIG. 26B depicts the levels of TNFα, and FIG. 26C depicts the levels of GM-CSF in the different groups of NK cells following stimulation. Prior to analysis NK cells were cultured in media supplemented with 40 IU of IL-2/mL.

FIGS. 27A-27B depict data related to the persistence of NK cells from two donors (donors 224 and 225 in FIGS. 27A and 27B, respectively) expressing the various NKG2D constructs 7, 14, and 21 days post-transduction. Prior to analysis NK cells were cultured in media supplemented with 40 IU of IL-2/mL.

FIGS. 28A-28B depict data related to the cytotoxicity of NK cells transduced with the indicated NKG2D constructs. NK cytotoxicity was measured against U2OS cells stably transduced to express Red Fluorescent Protein; U2OS cells were cultured with NK cells at a 1:4 and 1:2 E:T ratios (FIGS. 28A and 28B, respectively). Live U2OS cells were counted every 60 minutes for 72 hours using an Incucyte S3 Live-Cell Analysis System. Prior to analysis NK cells were cultured in media supplemented with 40 IU of IL-2/mL.

### DETAILED DESCRIPTION

### General

The emergence and persistence of aberrant cells (including virally infected and malignant cells) underlying many diseases is enabled by an insufficient immune response to said aberrant cells. A goal of immunotherapy is to initiate or augment the response of the patient's immune system, for example, to boost the ability of immune cells, such as Natural Killer (NK) cells to damage, kill, or otherwise inhibit damaged or diseased cells. One immunotherapy approach is the recombinant expression of chimeric receptors in immune cells for targeted recognition and destruction of the aberrant cells. In general, chimeric receptors comprise an extracellular receptor domain that recognizes ligands on target cells, an anchoring transmembrane domain, and an effector domain that transduces activating signals upon ligand binding. Some embodiments disclosed herein utilize chimeric receptors having that general structure, or having variations in that general structure. Additionally, in several embodiments, the transmembrane domain and the effector domain are separate peptides fused together. In several other embodiments, the transmembrane and the effector domain are derived from the same peptide. In some such embodiments, the transmembrane and effector domains comprise a single peptide (e.g., one peptide that passes through the membrane and is also poised to initiate a signaling cascade). As discussed in more detail below, truncations, mutations, additional linkers/spacer elements, dimers, and the like are used to generate chimeric receptor constructs that exhibit a desired degree of expression in an immune cell (e.g., an NK cell), induce cytotoxic activity from the NK cell, balanced with a degree of target avidity that avoids adverse effects on non-target cells. The recombinant expression of chimeric receptors as disclosed herein on the surface of immune cells can redirect the targeting of immune cells to aberrant cells of interest as well as augment the immune activation upon engagement.

### NK Cells for Immunotherapy

One immunotherapy approach involves administering to patients T cells engineered to express chimeric receptors to elicit a positive immune response. However, a drawback of this approach is that it necessitates the use of autologous cells to prevent the induction of graft-versus-host-disease in the patient. As is provided in several embodiments disclosed herein, compositions comprising engineered NK cells enjoy several advantages. For example, either autologous or donor-derived allogeneic cells can be employed with an NK cell approach. Additionally, according to several embodiments, the engineered NK cells as provided for herein do not significantly increase cytotoxicity against normal cells. Further, NK cells have a significant cytotoxic effect, once activated. In view of this, it is unexpected that the engineered NK cells as provided for herein, are able to further elevate that cytotoxic effect, thus providing an even more effective means of selectively killing diseased target cells. Accordingly, in several embodiments, there is provided a method of treating or preventing cancer or an infectious disease, comprising administering a therapeutically effective amount of NK cells expressing the chimeric receptors described herein. In one embodiment, the NK cells administered are autologous cells. In a further embodiment, the NK cells administered are donor-derived (allogeneic) cells.

In several embodiments, engagement and activation of a recombinant NK cell (e.g., by binding to a ligand on a target cell) expressing a chimeric receptor leads to the direct killing of the stressed and/or aberrant cell (e.g., tumor cells, virally-infected cells, etc.) by cytolysis. Accordingly, in several embodiments, there is provided a method of enhancing NK cell cytotoxicity, comprising administering NK cells engineered to express the chimeric receptors described herein. In one embodiment, the NK cells administered are autologous cells. In a further embodiment, the NK cells are donor-derived (allogenic) cells. In several embodiments, engineered NK cells lead to indirect destruction or inhibition of stressed and/or aberrant cell (e.g., tumor cells, virally-infected cells, etc.).

### Ligand Binding Domains

As mentioned above, in several embodiments NK cells recognize and destroy aberrant cells, including tumor cells and virally-infected cells. The cytotoxic activity of these innate immune cells is regulated by the balance of signaling from inhibitory and activating receptors, respectively, that reside on the cell surface. The former bind self-molecules expressed on the surface of healthy cells while the latter bind ligands expressed on aberrant cells. The increased engagement of activating receptors relative to inhibitory receptors leads to NK cell activation and target cell lysis. Natural killer Group 2 member D (NKG2D) is an important NK cell activating receptor that recognizes a number of ligands expressed on stressed and aberrant cells. The surface expression of various NKG2D ligands is generally low in healthy cells but is upregulated upon malignant transformation or viral infection. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells.

NKG2D's ability to recognize a plurality of surface markers of cell stress and infection make it a potentially useful component of a chimeric receptor-based immunotherapy approach. However, complicating the use of NKG2D as a chimeric receptor is its relationship with partner DAP10. NKG2D is a type II transmembrane glycoprotein that forms homodimers and assembles with two homodimers of DNAX-activating protein 10 (DAP10) to yield hexameric complexes on the membrane surface. This NKG2D-DAP10 association is necessary for both surface membrane expression of endogenous NKG2D as well as for transduction of the activation signal upon ligand binding. In several embodiments, a full length NKG2D is used. In one embodiment, full length NKG2D has the nucleic acid sequence of SEQ ID NO. 1. According to several embodiments disclosed herein, polynucleotides encoding chimeric receptors are provided wherein the extracellular receptor domain is a fragment of NKG2D that lacks its native transmembrane or intracellular domains yet advantageously retains its ability to bind native ligands of NKG2D, as well as transduce activation signals upon ligand binding. Thus, in several embodiments, the chimeric receptor encoded by the polypeptides disclosed herein does not comprise DAP10. In several embodiments, the NKG2D fragment is encoded by SEQ ID NO. 2. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO. 2, but retains, or in some embodiments, has enhanced, ligand-binding function. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO. 3. Additionally, in several embodiments signal peptides are used. The species or sequence of the signal peptide can vary with the construct. However, in several embodiments, a signal peptide derived from CD8 is used. In one embodiment, the signal peptide is from CD8α and has the sequence of SEQ ID NO. 4. In one embodiment, a sequence encoding a codon optimized NKG2D fragment comprises the sequence of SEQ ID NO. 68. In several embodiments, the fragment may have one or more additional mutations from SEQ ID NO. 68, but retains ligand-binding function. In several embodiments, the fragment may have one or more additional mutations from SEQ ID NO. 68, but has improved ligand-binding function.

### Transmembrane, Signaling and Combination Domains

As mentioned above, the general chimeric antigen receptor structure comprises at least one transmembrane domain, linking the ligand binding domain to a signaling domain(s). In several embodiments, however, a transmembrane domain can also serve to provide signaling function.

In several embodiments, the NKG2D fragment retains at least a portion of its normal transmembrane domain. In several embodiments, the transmembrane domain comprises at least a portion of CD8, which is a transmembrane glycoprotein normally expressed on both T cells and NK cells. In several embodiments, the transmembrane domain comprises CD8α, while in some embodiments CD8β is used. In several embodiments, the “hinge” of CD8α has the sequence of SEQ ID NO. 5. In several embodiments, the CD8α can be truncated or modified, such that it is at least 70%, at least 75%, at least 80%, at least 85%, at least 90%, at least 95% homologous with the CD8α having the sequence of SEQ ID NO. 5. In several embodiments, CD8β has the sequence of SEQ ID NO. 6. In several embodiments, the CD8β can be truncated or modified, such that it is at least 70%, at least 75%, at least 80%, at least 85%, at least 90%, at least 95% homologous with the CD8β having the sequence of SEQ ID NO. 6. In several embodiments, dimers of CD8α and CD8β are used.

In several embodiments, the transmembrane domain comprises CD16, which serves as a signaling domain as well. CD16 exists in two isoforms, a and b (also known as Fe gamma receptor IIIa and IIIb, respectively). These receptors normally bind to the Fc portion of IgG antibodies that in turn activates NK cells. Accordingly, in several embodiments, the transmembrane domain comprises CD16a, while in some embodiments CD16b is used. In several embodiments, CD16a has the sequence of SEQ ID NO. 7. In several embodiments, the CD16a can be truncated or modified, such that it is at least 70%, at least 75%, at least 80%, at least 85%, at least 90%, at least 95% homologous with the CD16a having the sequence of SEQ ID NO. 7. In several embodiments, CD16b has the sequence of SEQ ID NO. 8. In several embodiments, the CD16b can be truncated or modified, such that it is at least 70%, at least 75%, at least 80%, at least 85%, at least 90%, at least 95% homologous with the CD16b having the sequence of SEQ ID NO. 8. In several embodiments, dimers of CD16a and CD16b are used. In several embodiments the modifications to the CD16 transmembrane domain comprise additional nucleic acid residues to increase the length of the domain. Alternatively, CD16 may be shortened. The modifications to the length of CD16 advantageously can facilitate enhanced ligand-receptor interactions.

In several embodiments, the chimeric receptor comprises the Natural Killer Receptor 2B4 domain (referred to herein as “2B4”, and also known as CD244), which serves as a signaling domain as well. 2B4 is expressed on NK cells and regulates non-major histocompatibility complex (MHC) restricted killing through interactions between this receptor and its ligands on target cells. In several embodiments, the transmembrane domain comprises 2B4, while in several embodiments the 2B4 domain is an intracellular signaling domain. In several embodiments, 2B4 has the sequence of SEQ ID NO. 9. In several embodiments, the 2B4 can be truncated or modified, such that it is at least 70%, at least 75%, at least 80%, at least 85%, at least 90%, at least 95% homologous with the 2B4 having the sequence of SEQ ID NO. 9. In several embodiments, 2B4 is used as the sole transmembrane/signaling domain in the construct, however, in several embodiments, 2B4 can be used with one or more other domains. For example, combinations of CD16, 4-1BB, and/or 2B4 are used in some embodiments.

In some embodiments, signaling is achieved through DAP10, as mentioned above. In several embodiments, the fragment of NKG2D associates with DAP10 to provide pro-cytotoxic signals to the NK cell. In several embodiments, dimers of DAP10 are used. In several embodiments, the transmembrane domain comprises DAP10. In several embodiments, DAP10 has the sequence of SEQ ID NO. 10. In several embodiments, DAP10 can be truncated or modified, such that it is at least 70%, at least 75%, at least 80%, at least 85%, at least 90%, at least 95% homologous with the DAP10 having the sequence of SEQ ID NO. 10. Similarly, in some embodiments, DAP12 can be used, as it can also transduce such signals. In several embodiments, DAP12 has the sequence of SEQ ID NO. 11. In several embodiments, DAP12 can be truncated or modified, such that it is at least 70%, at least 75%, at least 80%, at least 85%, at least 90%, at least 95% homologous with the DAP12 having the sequence of SEQ ID NO. 11. In several embodiments, heterodimers of DAP10 and DAP12 are used.

In several embodiments, signaling is provided through 4-1BB (also known as CD137 and tumor necrosis factor receptor superfamily member 9 (TNFRSF 9)). 4-1BB is a co-stimulatory immune checkpoint molecule, typically functioning as a stimulatory molecule for activated T cells (e.g., crosslinking of 4-1BB enhances T cell proliferation and cytolytic activity). However, in several embodiments, the function of 4-1BB is advantageously used in conjunction with NK cells. In several embodiments, 4-1BB has the sequence of SEQ ID NO. 12. In several embodiments, 4-1BB can be truncated or modified, such that it is at least 70%, at least 75%, at least 80%, at least 85%, at least 90%, at least 95% homologous with the 4-1BB having the sequence of SEQ ID NO. 12. In several embodiments, 4-1BB is the sole signaling domain, but as discussed above, in several embodiments, 4-1BB functions unexpectedly well in combination with one or more of the other transmembrane/signaling domains disclosed herein. For example, in several embodiments, CD16 in conjunction with 4-1BB provides synergistic stimulation effects, resulting in particularly effective (e.g., cytotoxic) NK cells. In several embodiments, DAP10 in conjunction with 4-1BB provides synergistic stimulation effects, resulting in particularly effective (e.g., cytotoxic) NK cells. In several embodiments, DAP10 in conjunction with 4-1BB and/or 2B4 provides synergistic stimulation effects, resulting in particularly effective (e.g., cytotoxic) NK cells. Other improved characteristics result, in several embodiments, such as improved expression, improved persistence, and the like.

In several embodiments, the signaling domain comprises at least a portion of the CD3 T cell receptor complex. The T cell receptor complex comprises multiple subunits, including the zeta, alpha, beta, gamma, delta, and epsilon subunits. In several embodiments, the NK cells engineered according to several embodiments disclosed herein comprise at least one of these subunits (or a fragment thereof). In several embodiments, the signaling domain comprises the CD3 zeta subunit. In several embodiments, CD3 zeta has the sequence of SEQ ID NO. 13. In several embodiments, CD3 zeta can be truncated or modified, such that it is at least 70%, at least 75%, at least 80%, at least 85%, at least 90%, at least 95% homologous with the CD3 zeta having the sequence of SEQ ID NO. 13. In several embodiments, the CD3 zeta is mutated (e.g., amino acid mutations, insertions, or deletions) such that the domain no longer is consistent with the canonical immunoreceptor tyrosine-based activation motif or ITAM motif. Thus, in several embodiments, the NK cells comprise an engineered receptor that does not contain an ITAM motif. In some embodiments, the resultant engineered NK cells exhibit particularly enhanced cytotoxicity against target cells, with limited or reduced adverse side effects. This, in several embodiments, results from the synergistic interactions of the various portions of the chimeric receptor that are used in that given embodiment. In several embodiments, CD3zeta in conjunction with 4-1BB provides synergistic stimulation effects, resulting in particularly effective (e.g., cytotoxic) NK cells. In several embodiments, CD3zeta in conjunction with 2B4 provides synergistic stimulation effects, resulting in particularly effective (e.g., cytotoxic) NK cells. In several embodiments, CD3zeta in combination with 2B4 and 4-1BB provides synergistic stimulation effects, resulting in particularly effective (e.g., cytotoxic) NK cells. In several embodiments, the chimeric receptors leverage the dimerization of CD3zeta via its transmembrane domain. Thus, in several embodiments, the transmembrane domain comprises the CD3zeta transmembrane domain (or a fragment thereof). In some embodiments, 1, 2, 3, 4, 5, 6 or more extracellular CD3zeta residues (the “juxta-membrane portion”) are directly adjacent to the CD3zeta transmembrane domain. In some embodiments, CD3zeta transmembrane domain has the sequence of SEQ ID NO. 69. In several embodiments, the CD3zeta transmembrane domain can be truncated or modified, such that it is at least 70%, at least 75%, at least 80%, at least 85%, at least 90%, at least 95% homologous with the CD3zeta transmembrane domain having the sequence of SEQ ID NO. 69. In several embodiments the modifications to the CD3zeta transmembrane domain comprise additional nucleic acid residues to increase the length of the domain. In several embodiments, the CD3zeta transmembrane domain and CD3zeta juxta-membrane portion recruits full-length CD3zeta molecule to the synapse. In several embodiments, the recruitment of native CD3zeta to the engineered receptor (as compared to a receptor without a CD3zeta transmembrane domain) is increased by about 20%, by about 30%, by about 40% by about 50%, or more, depending on the embodiment. In several embodiments, the CD3zeta transmembrane domain is coupled to an effector domain comprising one or more of CD16, NCR1, NCR2, NCR3, 4-1BB, NKp80, FcRγ, CD3zeta and 2B4.

In several embodiments, the chimeric receptor comprises a CD28 domain. In several embodiments, the transmembrane domain comprises CD28, while in several embodiments the CD28 domain is an intracellular signaling domain, while in several embodiments the CD28 domain is a transmembrane/intracellular signaling domain. In several embodiments, the CD28 transmembrane domain has the sequence of SEQ ID NO. 105. In several embodiments, the CD28 transmembrane domain can be truncated or modified, such that it is at least 70%, at least 75%, at least 80%, at least 85%, at least 90%, at least 95% homologous with the CD28 having the sequence of SEQ ID NO. 105. In several embodiments, the CD28 intracellular signaling domain has the sequence of SEQ ID NO. 106. In several embodiments, the CD28 intracellular signaling domain can be truncated or modified, such that it is at least 70%, at least 75%, at least 80%, at least 85%, at least 90%, at least 95% homologous with the CD28 having the sequence of SEQ ID NO. 106. In several embodiments, CD28 is used as the sole transmembrane/signaling domain in the construct, however, in several embodiments, CD28 can be used with one or more other domains. For example, combinations of CD28, OX40, 4-1BB, and/or CD3zeta are used in some embodiments.

In several embodiments, the chimeric receptor comprises an OX40 domain. In several embodiments the OX40 domain is an intracellular signaling domain. In several embodiments, the OX40 intracellular signaling domain has the sequence of SEQ ID NO. 107. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO. 107. In several embodiments, OX40 is used as the sole transmembrane/signaling domain in the construct, however, in several embodiments, OX40 can be used with one or more other domains. For example, combinations of CD28, OX40, 4-1BB, and/or CD3zeta are used in some embodiments.

In still further embodiments, the signaling portion of the chimeric receptor comprises a portion of an ITAM, for example a hemi-tam. In several embodiments, these portions do not make up the canonical ITAM sequence, but rather comprise a portion that still can convey the signal required for NK cell cytotoxicity. In several embodiments, the hemi-tam has the sequence of SEQ ID NO. 14 (wherein X can be any residue). In several embodiments, the hemi-tam can be truncated or modified, such that it is at least 70%, at least 75%, at least 80%, at least 85%, at least 90%, at least 95% homologous with the hemi-tam having the sequence of SEQ ID NO. 14. In several embodiments, the chimeric receptor construct comprises the hemi-tam of SEQ ID NO. 14. In several embodiments, multiple hemi-tams can be used, for example in a head to tail, tail to head, head to head, or tail to tail configuration. In several embodiments, the presence of at least on hemi-tam confers enhanced signaling and cytotoxicity to the NK cells comprising a chimeric receptor employing the at least one hemi-tam. As discussed in more detail below, in several chimeric receptor comprises NKp80, which is one nonlimiting example of a hemi-tam.

In several embodiments, additional signaling regions are used, including, for example, signaling regions derived from receptors of the signaling lymphocytic activation molecule (SLAM) family. These receptors include, but are not limited to 2B4 (discussed above). Receptors of the SLAM family share a consensus motif that is tyrosine-based, in their cytoplasmic tails. That motif is S/TxYxxL/I, which are referred to as immunoreceptor tyrosine-based switch motifs (ITSM) (SEQ ID NO. 15). These receptors transmit activation signals through the SLAM-associated protein (SAP, encoded by the gene SH2D1A), which recruits the tyrosine kinase Fyn. Thus, according to several embodiments, the signaling region comprise a polypeptide sequence (or the nucleic acid encoding the same) comprising an ITSM motif. In several embodiments, the ITSM motif need not be fully encoded, but the signaling region is able to transmit an activation signal through SAP (or another similar pathway). In several embodiments, the ITSM motif has the sequence of SEQ ID NO. 15 (wherein X can be any amino acid residue). In several embodiments, the ITSM motif can be truncated or modified, such that it is at least 70%, at least 75%, at least 80%, at least 85%, at least 90%, at least 95% homologous with the ITSM motif having the sequence of SEQ ID NO. 15. In several embodiments, the ITSM motif comprises the sequence of SEQ ID NO. 15.

In addition to these variations in the NKG2D receptor, the transmembrane domain and signaling domain (and the combination transmembrane/signaling domains), additional co-activating molecules can be provided, in several embodiments. For example, in several embodiments, the NK cells are engineered to express membrane-bound interleukin 15 (mbIL15). In such embodiments, the presence of the mbIL15 on the NK cell function to further enhance the cytotoxic effects of the NK cell by synergistically enhancing the proliferation and longevity of the NK cells. In several embodiments, mbIL15 has the nucleic acid sequence of SEQ ID NO. 16. In several embodiments, mbIL15 can be truncated or modified, such that it is at least 70%, at least 75%, at least 80%, at least 85%, at least 90%, at least 95% homologous with the sequence of SEQ ID NO. 16. In several embodiments, the mbIL15 has the amino acid sequence of SEQ ID NO. 17. In conjunction with the chimeric receptors disclosed herein, such embodiments provide particularly effective NK cell compositions for targeting and destroying particular target cells.

### Chimeric Receptor Constructs

In view of the disclosure provided herein, there are a variety of chimeric receptors that can be generated and expressed in NK cells in order to target and destroy particular target cells, such as diseased or cancerous cells. Non-limiting examples of such chimeric receptors are discussed in more detail below.

As discussed above, portions of the T cell receptor complex, in particular CD3zeta, serve as potent activators of immune signaling cascades. Likewise, the receptor 4-1BB, a tumor necrosis factor superfamily member, activates NK cells upon ligand binding. In several embodiments, these two signaling components act in a synergistic manner to activate NK cells upon binding of a ligand to the chimeric receptor. Thus, in several embodiments, there are provided polynucleotides encoding a NKG2D/CD8α/4-1BB/CD3zeta chimeric receptor, which comprises an NKG2D fragment extracellular receptor domain that binds native ligands of NKG2D, a CD8 transmembrane region, and an effector domain comprising the signaling domains of 4-1BB and CD3zeta. In one embodiment, this chimeric receptor is encoded by the nucleic acid sequence of SEQ ID NO: 18. In one embodiment, this chimeric receptor is encoded by the nucleic acid sequence of SEQ ID NO: 108. In yet another embodiment, the NKG2D/CD8α/4-1BB/CD3zeta chimeric receptor comprises the amino acid sequence of SEQ ID NO: 19. In several embodiments, this construct is particularly efficacious when the NK cells concurrently express mbIL15, the mbIL15 provides a further synergistic effect with respect to the activation and cytotoxic nature of the NK cells. In some embodiments, the sequence of the chimeric receptor may vary from SEQ ID NO. 18 (such as, for example, SEQ ID NO: 108), but remains, depending on the embodiment, at least 70%, at least 75%, at least 80%, at least 85%, at least 90%, or at least 95% homologous with SEQ ID NO. 18. In several embodiments, while the chimeric receptor may vary from SEQ ID NO. 18 (such as, for example, SEQ ID NO: 108), the chimeric receptor retains, or in some embodiments, has enhanced, NK cell activating and/or cytotoxic function.

The receptor 2B4 possesses several immunoreceptor tyrosine-based switch motifs (ITSMs) and has the potential to transduce activating signals. Likewise, signaling through the receptor 4-1BB, a tumor necrosis factor superfamily member, also activates NK cells upon ligand binding. Thus, capitalizing on the ability of these signaling molecules to cooperate to generate unexpectedly effectively cytotoxic NK cells, in several embodiments, there are provided polynucleotides encoding a NKG2D/CD8α/2B4/4-1BB chimeric receptor, which comprises an NKG2D fragment extracellular receptor domain that binds native ligands of NKG2D, a CD8α transmembrane region, and an effector domain comprising the signaling domains of 4-1BB and 2B4. Additionally, in several embodiments, this construct can optionally be co-expressed with mbIL15.

In several embodiments, combinations of 2B4 with CD3zeta are used with NK cells to generate enhanced cytotoxicity against target cells. Thus, in several embodiments, there are provided polynucleotides encoding a NKG2D/CD88a/2B4/CD3zeta chimeric receptor, which comprises an NKG2D fragment extracellular receptor domain that binds native ligands of NKG2D, a CD8α transmembrane region, and an effector domain comprising the signaling domains of CD3zeta and 2B4. Additionally, in several embodiments, this construct can optionally be co-expressed with mbIL15. As discussed above, 4-1BB, like CD3zeta and 2B4, can function as a potent activator of immune signaling cascades. In several embodiments, these three signaling components act in a synergistic manner to activate NK cells upon binding of a ligand to the chimeric receptor. Thus, in several embodiments, there are provided polynucleotides encoding a NKG2D/CD8α/4-1BB/2B4/CD3zeta chimeric receptor, which comprises an NKG2D fragment extracellular receptor domain that binds native ligands of NKG2D, a CD8 transmembrane region, and an effector domain comprising the signaling domains of 4-1BB, 2B4 and CD3zeta. In one embodiment, this chimeric receptor is encoded by the nucleic acid sequence of SEQ ID NO: 58. In yet another embodiment, the NKG2D/CD8α/4-1BB/CD3zeta chimeric receptor comprises the amino acid sequence of SEQ ID NO: 59. In several embodiments, this construct is particularly efficacious when the NK cells concurrently express mbIL15, the mbIL15 provides a further synergistic effect with respect to the activation and/or cytotoxic nature of the NK cells. In some embodiments, the sequence of the chimeric receptor may vary from SEQ ID NO. 58, but remains, depending on the embodiment, at least 70%, at least 75%, at least 80%, at least 85%, at least 90%, or at least 95% homologous with SEQ ID NO. 58. In several embodiments, while the chimeric receptor may vary from SEQ ID NO. 58, the chimeric receptor retains, or in some embodiments, has enhanced, NK cell activating and/or cytotoxic function.

In several alternative embodiments, there are provided polynucleotides encoding a NKG2D/CD8α/DAP10/4-1BB chimeric receptor, which comprises an NKG2D fragment extracellular receptor domain that binds native ligands of NKG2D, a CD8α transmembrane region, and an effector domain comprising the signaling domains of 4-1BB and DAP10. In one embodiment, this chimeric receptor is encoded by the nucleic acid sequence of SEQ ID NO: 60. In yet another embodiment, the NKG2D/CD8α/4-1BB/DAP10 chimeric receptor comprises the amino acid sequence of SEQ ID NO: 61. Additionally, in several embodiments, this construct can optionally be co-expressed with mbIL15. In several embodiments, this construct is particularly efficacious when the NK cells concurrently express mbIL15, the mbIL15 provides a further synergistic effect with respect to the activation and cytotoxic nature of the NK cells. In some embodiments, the sequence of the chimeric receptor may vary from SEQ ID NO. 60, but remains, depending on the embodiment, at least 70%, at least 75%, at least 80%, at least 85%, at least 90%, or at least 95% homologous with SEQ ID NO. 60. In several embodiments, while the chimeric receptor may vary from SEQ ID NO. 60, the chimeric receptor retains, or in some embodiments, has enhanced, NK cell activating and/or cytotoxic function. Further, as discussed above, 2B4, like DAP10 and 4-1BB, is a potent activator of immune signaling cascades. In several embodiments, these three signaling components act in a synergistic manner to activate NK cells upon binding of a ligand to the chimeric receptor. Thus, in several embodiments, there are provided polynucleotides encoding a NKG2D/CD8α/4-1BB/DAP10/2B4 chimeric receptor, which comprises an NKG2D fragment extracellular receptor domain that binds native ligands of NKG2D, a CD8 transmembrane region, and an effector domain comprising the signaling domains of 4-1BB, 2B4 and DAP10, wherein 4-1BB is followed by DAP10, and DAP10 is followed by 2B4. In one embodiment, this chimeric receptor is encoded by the nucleic acid sequence of SEQ ID NO: 62. In yet another embodiment, the NKG2D/CD8α/4-1BB/CD3zeta chimeric receptor comprises the amino acid sequence of SEQ ID NO: 63. In several embodiments, this construct is particularly efficacious when the NK cells concurrently express mbIL15, the mbIL15 provides a further synergistic effect with respect to the activation and cytotoxic nature of the NK cells. In some embodiments, the sequence of the chimeric receptor may vary from SEQ ID NO. 62, but remains, depending on the embodiment, at least 70%, at least 75%, at least 80%, at least 85%, at least 90%, or at least 95% homologous with SEQ ID NO. 62. In several embodiments, while the chimeric receptor may vary from SEQ ID NO. 62, the chimeric receptor retains, or in some embodiments, has enhanced, NK cell activating and/or cytotoxic function. In several other embodiments, there are provided polynucleotides encoding a NKG2D/CD8α/4-1BB/2B4/DAP10 chimeric receptor, which comprises an NKG2D fragment extracellular receptor domain that binds native ligands of NKG2D, a CD8 transmembrane region, and an effector domain comprising the signaling domains of 4-1BB, 2B4 and DAP10, wherein 4-1BB is followed by 2B4, and 2B4 is followed by DAP10. In one embodiment, this chimeric receptor is encoded by the nucleic acid sequence of SEQ ID NO: 64. In yet another embodiment, the NKG2D/CD8α/4-1BB/CD3zeta chimeric receptor comprises the amino acid sequence of SEQ ID NO: 65. In several embodiments, this construct is particularly efficacious when the NK cells concurrently express mbIL15, the mbIL15 provides a further synergistic effect with respect to the activation and cytotoxic nature of the NK cells. In some embodiments, the sequence of the chimeric receptor may vary from SEQ ID NO. 64, but remains, depending on the embodiment, at least 70%, at least 75%, at least 80%, at least 85%, at least 90%, or at least 95% homologous with SEQ ID NO. 64. In several embodiments, while the chimeric receptor may vary from SEQ ID NO. 64, the chimeric receptor retains, or in some embodiments, has enhanced, NK cell activating and/or cytotoxic function.

In several additional embodiments, transmembrane and effector domains (and associated function) of the chimeric receptor are derived from the same peptide. CD16 is a potent activating receptor expressed on the surface of NK cells. Thus, in several embodiments, polynucleotides are provided encoding a NKG2D/CD16 chimeric receptor, which comprises an NKG2D fragment extracellular receptor domain that binds native ligands of NKG2D and a CD16 peptide comprising both the transmembrane region and intracellular effector domain. In one embodiment, this chimeric receptor comprises the nucleic acid sequence of SEQ ID NO: 23. In yet another embodiment, this chimeric receptor is encoded by the amino acid sequence of SEQ ID NO: 24. In some embodiments, the sequence of the chimeric receptor may vary from SEQ ID NO. 23, but remains, depending on the embodiment, at least 70%, at least 75%, at least 80%, at least 85%, at least 90%, or at least 95% homologous with SEQ ID NO. 23. In several embodiments, while the chimeric receptor may vary from SEQ ID NO. 23, the chimeric receptor retains, or in some embodiments, has enhanced, NK cell activating and/or cytotoxic function. Additionally, in several embodiments, this construct can optionally be co-expressed with mbIL15.

In several additional embodiments, polynucleotides are provided encoding a NKG2D/CD16/4-1BB chimeric receptor, wherein the signaling domain of 4-1BB acts as a second transducer of activating signals in the effector domain. Additionally, in several embodiments, this construct can optionally be co-expressed with mbIL15.

CD3zeta dimerizes via its transmembrane domain. Thus, in several embodiments, chimeric receptors are provided wherein a CD3zeta transmembrane domain recruits full-length CD3zeta molecule to the synapse. In several embodiments, there are provided polynucleotides encoding a chimeric receptor which comprises a NKG2D fragment that binds native ligands of NKG2D, a CD8α hinge, 0, 1, 2, 3, 4, 5, 6 or more extracellular CD3zeta residues (the “juxta-membrane portion”) directly adjacent to a CD3zeta transmembrane domain, and an effector domain comprising one or more of CD16, NCR1, NCR2, NCR3, 4-1BB, NKp80, FcRγ, CD3zeta and 2B4.

In several embodiments, chimeric receptors are provided wherein a CD3zeta transmembrane domain is coupled to an effector domain comprising one or both of 4-1BB and CD16. Thus, in several embodiments, polynucleotides are provided encoding a NKG2D/CD3zetaTM/4-1BB chimeric receptor, which comprises a fragment of NKG2D that is codon optimized coupled to a CD8α hinge, a CD3zeta transmembrane region, and an effector domain comprising 4-1BB. In one embodiment, this chimeric receptor comprises the nucleic acid sequence of SEQ ID NO: 78. In yet another embodiment, this chimeric receptor is encoded by the amino acid sequence of SEQ ID NO: 79. In some embodiments, the sequence of the chimeric receptor may vary from SEQ ID NO. 78, but remains, depending on the embodiment, at least 70%, at least 75%, at least 80%, at least 85%, at least 90%, or at least 95% homologous with SEQ ID NO. 78. In several embodiments, while the chimeric receptor may vary from SEQ ID NO. 78, the chimeric receptor retains, or in some embodiments, has enhanced, NK cell activating and/or cytotoxic function. Additionally, in several embodiments, this construct can optionally be co-expressed with mbIL15.

In several embodiments, polynucleotides are provided encoding a NKG2D/CD3zetaTM/CD16/4-1BB chimeric receptor, which comprises a fragment of NKG2D that is codon optimized coupled to a CD8α hinge, a CD3zeta transmembrane region, and an effector domain comprising CD16 followed by 4-1BB. In one embodiment, this chimeric receptor comprises the nucleic acid sequence of SEQ ID NO: 70. In yet another embodiment, this chimeric receptor is encoded by the amino acid sequence of SEQ ID NO: 71. In some embodiments, the sequence of the chimeric receptor may vary from SEQ ID NO. 70, but remains, depending on the embodiment, at least 70%, at least 75%, at least 80%, at least 85%, at least 90%, or at least 95% homologous with SEQ ID NO. 70. In several embodiments, while the chimeric receptor may vary from SEQ ID NO. 70, the chimeric receptor retains, or in some embodiments, has enhanced, NK cell activating and/or cytotoxic function. Additionally, in several embodiments, this construct can optionally be co-expressed with mbIL15. Further, in several embodiments, polynucleotides are provided encoding a NKG2D/CD3zetaTM/4-1BB/CD16 chimeric receptor, which comprises a fragment of NKG2D that is codon optimized coupled to a CD8α hinge, a CD3zeta transmembrane region, and an effector domain comprising 4-1BB followed by CD16. In some embodiments, the effector domain further comprises a GS3 linker. In some embodiments, the GS3 linker is positioned between 4-1BB and CD16. In one embodiment, this chimeric receptor comprises the nucleic acid sequence of SEQ ID NO: 84. In yet another embodiment, this chimeric receptor is encoded by the amino acid sequence of SEQ ID NO: 85. In some embodiments, the sequence of the chimeric receptor may vary from SEQ ID NO. 84, but remains, depending on the embodiment, at least 70%, at least 75%, at least 80%, at least 85%, at least 90%, or at least 95% homologous with SEQ ID NO. 84. In several embodiments, while the chimeric receptor may vary from SEQ ID NO. 84, the chimeric receptor retains, or in some embodiments, has enhanced, NK cell activating and/or cytotoxic function. Additionally, in several embodiments, this construct can optionally be co-expressed with mbIL15. Further, in several embodiments, polynucleotides are provided encoding a NKG2D×2/CD3zetaTM/CD16/4-1BB chimeric receptor, which comprises the fragment of NKG2D that is codon optimized coupled to a GS3 linker, an additional NKG2D fragment, a CD8α hinge, a CD3zeta transmembrane region, and an effector domain comprising a CD16 and 4-1BB. In one embodiment, this chimeric receptor comprises the nucleic acid sequence of SEQ ID NO: 72. In yet another embodiment, this chimeric receptor is encoded by the amino acid sequence of SEQ ID NO: 73. In some embodiments, the sequence of the chimeric receptor may vary from SEQ ID NO. 72, but remains, depending on the embodiment, at least 70%, at least 75%, at least 80%, at least 85%, at least 90%, or at least 95% homologous with SEQ ID NO. 72. In several embodiments, while the chimeric receptor may vary from SEQ ID NO. 72, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein a CD3zeta transmembrane domain is coupled to an effector domain comprising NKp80. Thus, in several embodiments, polynucleotides are provided encoding a NKG2D/CD3zetaTM/CD16/4-1BB/NKp80 chimeric receptor, which chimeric receptor comprises a fragment of NKG2D coupled to a CD8α hinge, a CD3zeta transmembrane region, and an effector domain comprising a CD16, 4-1BB, and NKp80. In some embodiments, the effector domain further comprises a GS3 linker. In some embodiments, the GS3 linker is positioned between 4-1BB and NKp80. In one embodiment, this chimeric receptor comprises the nucleic acid sequence of SEQ ID NO: 74. In yet another embodiment, this chimeric receptor is encoded by the amino acid sequence of SEQ ID NO: 75. In some embodiments, the sequence of the chimeric receptor may vary from SEQ ID NO. 74, but remains, depending on the embodiment, at least 70%, at least 75%, at least 80%, at least 85%, at least 90%, or at least 95% homologous with SEQ ID NO. 74. In several embodiments, while the chimeric receptor may vary from SEQ ID NO. 74, the chimeric receptor retains, or in some embodiments, has enhanced, NK cell activating and/or cytotoxic function. Additionally, in several embodiments, this construct can optionally be co-expressed with mbIL15. Further, in several embodiments, polynucleotides are provided encoding a 2×NKG2D/CD3zetaTM/CD16/4-1BB/NKp80 chimeric receptor, which comprises the fragment of NKG2D that is codon optimized coupled to a GS3 linker, an additional NKG2D fragment, a CD8α hinge, a CD3zeta transmembrane region, and an effector domain comprising a CD16, 4-1BB, and NKp80. In some embodiments, the effector domain further comprises a GS3 linker. In some embodiments, the GS3 linker is positioned between 4-1BB and NKp80. In one embodiment, this chimeric receptor comprises the nucleic acid sequence of SEQ ID NO: 76. In yet another embodiment, this chimeric receptor is encoded by the amino acid sequence of SEQ ID NO: 77. In some embodiments, the sequence of the chimeric receptor may vary from SEQ ID NO. 76, but remains, depending on the embodiment, at least 70%, at least 75%, at least 80%, at least 85%, at least 90%, or at least 95% homologous with SEQ ID NO. 76. In several embodiments, while the chimeric receptor may vary from SEQ ID NO. 76, the chimeric receptor retains, or in some embodiments, has enhanced, NK cell activating and/or cytotoxic function. Additionally, in several embodiments, this construct can optionally be co-expressed with mbIL15. Further, in several embodiments, polynucleotides are provided encoding a NKG2D/CD3zetaTM/4-1BB/NKp80 chimeric receptor, which comprises a fragment of NKG2D that is codon optimized coupled to a CD8α hinge, a CD3zeta transmembrane region, and an effector domain comprising 4-1BB and NKp80. In some embodiments, the effector domain further comprises a GS3 linker. In some embodiments, the GS3 linker is positioned between 4-1BB and NKp80. In one embodiment, this chimeric receptor comprises the nucleic acid sequence of SEQ ID NO: 82. In yet another embodiment, this chimeric receptor is encoded by the amino acid sequence of SEQ ID NO: 83. In some embodiments, the sequence of the chimeric receptor may vary from SEQ ID NO. 82, but remains, depending on the embodiment, at least 70%, at least 75%, at least 80%, at least 85%, at least 90%, or at least 95% homologous with SEQ ID NO. 82. In several embodiments, while the chimeric receptor may vary from SEQ ID NO. 82, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein a CD3zeta transmembrane domain is coupled to an effector domain comprising CD3zeta. Thus, in several embodiments, polynucleotides are provided encoding a NKG2D/CD3zetaTM/4-1BB/CD3zeta chimeric receptor, which comprises a fragment of NKG2D that is codon optimized coupled to a CD8α hinge, a CD3zeta transmembrane region, and an effector domain comprising 4-1BB and CD3zeta. In one embodiment, this chimeric receptor comprises the nucleic acid sequence of SEQ ID NO: 80. In yet another embodiment, this chimeric receptor is encoded by the amino acid sequence of SEQ ID NO: 81. In some embodiments, the sequence of the chimeric receptor may vary from SEQ ID NO. 80, but remains, depending on the embodiment, at least 70%, at least 75%, at least 80%, at least 85%, at least 90%, or at least 95% homologous with SEQ ID NO. 80. In several embodiments, while the chimeric receptor may vary from SEQ ID NO. 80, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein a CD3zeta transmembrane domain is coupled to an effector domain comprising FcRγ. Thus, in several embodiments, polynucleotides are provided encoding a NKG2D/CD3zetaTM/4-1BB/FcRγ chimeric receptor, which comprises a fragment of NKG2D coupled to a CD8α hinge, a CD3zeta transmembrane region, and an effector domain comprising 4-1BB and FcRγ. In one embodiment, this chimeric receptor comprises the nucleic acid sequence of SEQ ID NO: 86. In yet another embodiment, this chimeric receptor is encoded by the amino acid sequence of SEQ ID NO: 87. In some embodiments, the sequence of the chimeric receptor may vary from SEQ ID NO. 86, but remains, depending on the embodiment, at least 70%, at least 75%, at least 80%, at least 85%, at least 90%, or at least 95% homologous with SEQ ID NO. 86. In several embodiments, while the chimeric receptor may vary from SEQ ID NO. 86, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein a CD3zeta transmembrane domain is coupled to an effector domain comprising CD28. Thus, in several embodiments, polynucleotides are provided encoding a NKG2D/CD3zetaTM/CD28/CD3zeta chimeric receptor, which comprises an NKG2D fragment extracellular receptor domain that binds native ligands of NKG2D, a CD8α hinge, a CD3zeta transmembrane region, and intracellular effector domain comprising CD28 and CD3zeta. In one embodiment, this chimeric receptor comprises the nucleic acid sequence of SEQ ID NO: 102. In yet another embodiment, this chimeric receptor is encoded by the amino acid sequence of SEQ ID NO: 103. In some embodiments, the sequence of the chimeric receptor may vary from SEQ ID NO. 102, but remains, depending on the embodiment, at least 70%, at least 75%, at least 80%, at least 85%, at least 90%, or at least 95% homologous with SEQ ID NO. 102. In several embodiments, while the chimeric receptor may vary from SEQ ID NO. 102, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein the extracellular domain comprises a fragment of NKG2D coupled IL15. Thus, in several embodiments, polynucleotides are provided encoding an IL15/NKG2D/CD8α/4-1BB/CD3zeta chimeric receptor, which comprises an NKG2D fragment extracellular receptor domain that binds native ligands of NKG2D linked to IL-15, a CD8α hinge, a CD8α transmembrane domain, and intracellular effector domain comprising 4-1BB and CD3zeta. In some embodiments, the extracellular domain further comprises a GS3 linker. In some embodiments, the GS3 linker is positioned between IL15 and the NKG2D fragment extracellular receptor domain. In one embodiment, this chimeric receptor comprises the nucleic acid sequence of SEQ ID NO: 88. In yet another embodiment, this chimeric receptor is encoded by the amino acid sequence of SEQ ID NO: 89. In some embodiments, the sequence of the chimeric receptor may vary from SEQ ID NO. 88, but remains, depending on the embodiment, at least 70%, at least 75%, at least 80%, at least 85%, at least 90%, or at least 95% homologous with SEQ ID NO. 88. In several embodiments, while the chimeric receptor may vary from SEQ ID NO. 88, the chimeric receptor retains, or in some embodiments, has enhanced, NK cell activating and/or cytotoxic function.

In several embodiments, chimeric receptors are provided wherein the extracellular domain comprises a fragment of NKG2D coupled to a IgG4 short hinge. Thus, in several embodiments, polynucleotides are provided encoding a NKG2D/IgG4/CD8α/4-1BB/CD3zeta chimeric receptor, which comprises an NKG2D fragment extracellular receptor domain that binds native ligands of NKG2D, an IgG4 short hinge, a CD8α transmembrane domain, and intracellular effector domain comprising 4-1BB, and CD3zeta. In one embodiment, this chimeric receptor comprises the nucleic acid sequence of SEQ ID NO: 96. In yet another embodiment, this chimeric receptor is encoded by the amino acid sequence of SEQ ID NO: 97. In some embodiments, the sequence of the chimeric receptor may vary from SEQ ID NO. 96, but remains, depending on the embodiment, at least 70%, at least 75%, at least 80%, at least 85%, at least 90%, or at least 95% homologous with SEQ ID NO. 96. In several embodiments, while the chimeric receptor may vary from SEQ ID NO. 96, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided wherein the effector domain comprises OX40. Thus, in several embodiments, polynucleotides are provided encoding a NKG2D/CD8α/OX40/CD3zeta chimeric receptor, which comprises an NKG2D fragment extracellular receptor domain that binds native ligands of NKG2D, a CD8α hinge, a CD8α transmembrane domain, and an intracellular effector domain comprising OX40, and CD3zeta. In one embodiment, this chimeric receptor comprises the nucleic acid sequence of SEQ ID NO: 90. In yet another embodiment, this chimeric receptor is encoded by the amino acid sequence of SEQ ID NO: 91. In some embodiments, the sequence of the chimeric receptor may vary from SEQ ID NO. 90, but remains, depending on the embodiment, at least 70%, at least 75%, at least 80%, at least 85%, at least 90%, or at least 95% homologous with SEQ ID NO. 90. In several embodiments, while the chimeric receptor may vary from SEQ ID NO. 90, the chimeric receptor retains, or in some embodiments, has enhanced, NK cell activating and/or cytotoxic function. Additionally, in several embodiments, this construct can optionally be co-expressed with mbTL15. In several embodiments, polynucleotides are provided encoding a NKG2D/IgG4/CD8α/OX40/CD3zeta chimeric receptor, which comprises an NKG2D fragment extracellular receptor domain that binds native ligands of NKG2D, an IgG4 hinge, a CD8α transmembrane domain, and intracellular effector domain comprising OX40 and CD3zeta. In one embodiment, this chimeric receptor comprises the nucleic acid sequence of SEQ ID NO: 100. In yet another embodiment, this chimeric receptor is encoded by the amino acid sequence of SEQ ID NO: 101. In some embodiments, the sequence of the chimeric receptor may vary from SEQ ID NO. 100, but remains, depending on the embodiment, at least 70%, at least 75%, at least 80%, at least 85%, at least 90%, or at least 95% homologous with SEQ ID NO. 100. In several embodiments, while the chimeric receptor may vary from SEQ ID NO. 100, the chimeric receptor retains, or in some embodiments, has enhanced, NK cell activating and/or cytotoxic function. Additionally, in several embodiments, this construct can optionally be co-expressed with mbIL15.

In several embodiments, chimeric receptors are provided comprising a CD28 peptide comprising both the transmembrane region and intracellular effector domain. Thus, in several embodiments, polynucleotides are provided encoding a NKG2D/CD28/CD3zeta chimeric receptor, which comprises an NKG2D fragment extracellular receptor domain that binds native ligands of NKG2D, a CD8α hinge, a CD28 transmembrane/intracellular domain, and CD3zeta. In one embodiment, this chimeric receptor comprises the nucleic acid sequence of SEQ ID NO: 92. In yet another embodiment, this chimeric receptor is encoded by the amino acid sequence of SEQ ID NO: 93. In some embodiments, the sequence of the chimeric receptor may vary from SEQ ID NO. 92, but remains, depending on the embodiment, at least 70%, at least 75%, at least 80%, at least 85%, at least 90%, or at least 95% homologous with SEQ ID NO. 92. In several embodiments, while the chimeric receptor may vary from SEQ ID NO. 92, the chimeric receptor retains, or in some embodiments, has enhanced, NK cell activating and/or cytotoxic function. Additionally, in several embodiments, this construct can optionally be co-expressed with mbIL15. In further embodiments, polynucleotides are provided encoding a NKG2D/CD28/CD3zeta/4-1BB chimeric receptor, which comprises an NKG2D fragment extracellular receptor domain that binds native ligands of NKG2D, a CD8α hinge, a CD28 transmembrane/intracellular domain, and 4-1BB and CD3zeta. In one embodiment, this chimeric receptor comprises the nucleic acid sequence of SEQ ID NO: 94. In yet another embodiment, this chimeric receptor is encoded by the amino acid sequence of SEQ ID NO: 95. In some embodiments, the sequence of the chimeric receptor may vary from SEQ ID NO. 94, but remains, depending on the embodiment, at least 70%, at least 75%, at least 80%, at least 85%, at least 90%, or at least 95% homologous with SEQ ID NO. 94. In several embodiments, while the chimeric receptor may vary from SEQ ID NO. 94, the chimeric receptor retains, or in some embodiments, has enhanced, NK cell activating and/or cytotoxic function. Additionally, in several embodiments, this construct can optionally be co-expressed with mbTL15. In further embodiments, polynucleotides are provided encoding a NKG2D/IgG4/CD28/CD3zeta chimeric receptor, which comprises an NKG2D fragment extracellular receptor domain that binds native ligands of NKG2D, an IgG4 hinge, a CD28 transmembrane/intracellular domain, and CD3zeta. In one embodiment, this chimeric receptor comprises the nucleic acid sequence of SEQ ID NO: 98. In yet another embodiment, this chimeric receptor is encoded by the amino acid sequence of SEQ ID NO: 99. In some embodiments, the sequence of the chimeric receptor may vary from SEQ ID NO. 98, but remains, depending on the embodiment, at least 70%, at least 75%, at least 80%, at least 85%, at least 90%, or at least 95% homologous with SEQ ID NO. 98. In several embodiments, while the chimeric receptor may vary from SEQ ID NO. 98, the chimeric receptor retains, or in some embodiments, has enhanced, NK cell activating and/or cytotoxic function. Additionally, in several embodiments, this construct can optionally be co-expressed with mbIL15.

NCR1 (NKp46), NCR2 (NKp44) and NCR3 (NKp30) are receptors on NK cells that transduce activation signals upon ligand binding. Thus, in several embodiments, polynucleotides are provided encoding a NKG2D/NCR1 chimeric receptor, which comprises an NKG2D fragment extracellular receptor domain that binds native ligands of NKG2D and a NCR1 peptide comprising both the transmembrane region and intracellular effector domain. In one embodiment, this chimeric receptor comprises the nucleic acid sequence of SEQ ID NO: 27. In yet another embodiment, this chimeric receptor is encoded by the amino acid sequence of SEQ ID NO: 28. In some embodiments, the sequence of the chimeric receptor may vary from SEQ ID NO. 30, but remains, depending on the embodiment, at least 70%, at least 75%, at least 80%, at least 85%, at least 90%, or at least 95% homologous with SEQ ID NO. 27. In several embodiments, while the chimeric receptor may vary from SEQ ID NO. 27, the chimeric receptor retains, or in some embodiments, has enhanced, NK cell activating and/or cytotoxic function. Additionally, in several embodiments, this construct can optionally be co-expressed with mbIL15.

In several additional embodiments, polynucleotides are provided encoding a NKG2D/NCR1/4-1BB chimeric receptor, wherein the signaling domain of 4-1BB acts as a second transducer of activating signals in the effector domain, leading to synergistically enhanced NK cell activation and cytotoxicity. In several additional embodiments, polynucleotides are provided encoding a NKG2D/NCR2 chimeric receptor, which comprises an NKG2D fragment extracellular receptor domain that binds native ligands of NKG2D and a NCR2 peptide comprising both the transmembrane region and intracellular effector domain. As with NCR1, in several embodiments these constructs are particularly amenable for use in creating NK cells expressing the chimeric receptor, due to their relatively small size and simplicity on sequence. However, they retain the ability, in several embodiments, to yield highly effective NK cells, despite the apparent simplicity of the construct. Additionally, in several embodiments, these constructs can optionally be co-expressed with mbIL15.

In several additional embodiments, polynucleotides are provided encoding a NKG2D/NCR3 chimeric receptor, which comprises an NKG2D fragment extracellular receptor domain that binds native ligands of NKG2D and a NCR3 peptide comprising both the transmembrane region and intracellular effector domain. As with NCR1 and or NCR2, in several embodiments these constructs are particularly amenable for use in creating NK cells expressing the chimeric receptor, due to their relatively small size and simplicity on sequence. However, they retain the ability, in several embodiments, to yield highly effective NK cells, despite the apparent simplicity of the construct. In one embodiment, this chimeric receptor comprises the nucleic acid sequence of SEQ ID NO: 29. In yet another embodiment, this chimeric receptor is encoded by the amino acid sequence of SEQ ID NO: 30. In some embodiments, the sequence of the chimeric receptor may vary from SEQ ID NO. 29, but remains, depending on the embodiment, at least 70%, at least 75%, at least 80%, at least 85%, at least 90%, or at least 95% homologous with SEQ ID NO. 29. In several embodiments, while the chimeric receptor may vary from SEQ ID NO. 29, the chimeric receptor retains, or in some embodiments, has enhanced, NK cell activating and/or cytotoxic function. Additionally, in several embodiments, this construct can optionally be co-expressed with mbIL15.

In several additional embodiments, polynucleotides are provided encoding a NKG2D/NCR2/4-1BB chimeric receptor, wherein the signaling domain of 4-1BB acts as a second transducer of activating signals in the effector domain, thereby leading to a synergistic effect between the signaling domains, and unexpectedly effectively cytotoxic NK cells. Additionally, in several embodiments, this construct can optionally be co-expressed with mbIL15.

In several additional embodiments, polynucleotides are provided encoding a NKG2D/NCR3/4-1BB chimeric receptor, wherein the signaling domain of 4-1BB acts as a second transducer of activating signals in the effector domain, thereby leading to a synergistic effect between the signaling domains, and unexpectedly effectively cytotoxic NK cells. Additionally, in several embodiments, this construct can optionally be co-expressed with mbIL15.

In some embodiments the surface expression and efficacy of the chimeric receptors disclosed herein are enhanced by variations in a spacer region (hinge), which, in several embodiments, are located in the extracellular domain between the NKG2D fragment and the transmembrane domain. In some embodiments, the hinge regions can be included between other portions of the chimeric receptor (e.g., between intracellular and transmembrane domains, or between multiple intracellular domains). In some embodiments, domains that serve certain purposes as disclosed elsewhere herein, can serve additional functions. For example, in several embodiments, CD8α is repurposed to serve as a hinge region (encoded, in several embodiments, by the nucleic acid sequence of SEQ ID NO: 5). In yet another embodiment, the hinge region comprises an N-terminal truncated form of CD8α and/or a C-terminal truncated form of CD8α. Depending on the embodiment, these truncations can be at least about 50%, at least about 60%, at least about 70%, at least about 80%, or at least about 90% homologous to the hinge encoded by SEQ ID NO. 5. In several additional embodiments, the hinge comprises spans of Glycine and Serine residues (herein termed “GS linkers”) where GSn represents the sequence (Gly-Gly-Gly-Gly-Ser)n (SEQ ID NO. 42). In one embodiment, the hinge comprises both CD8α and GS3, and is encoded by the amino acid sequence of SEQ ID NO: 32, for example, where n=3. In additional embodiments, the value of n may be equal to 1, 2, 4, 5, 6, 7, 8, 9, 10, 11, 12, 13, 14, 15 or greater depending on the embodiment. In several embodiments, the hinge could also be structured as GSn/CD8α. Alternatively, the GS linker can comprise the entire hinge region. In one such embodiment, the hinge region is encoded by the nucleic acid sequence of SEQ ID NO: 33. In another such embodiment, the hinge region is encoded by the nucleic acid sequence of SEQ ID NO: 34. In several embodiments, IgG4 is repurposed as a hinge region (encoded, in several embodiments, by the nucleic acid sequence of SEQ ID NO: 104). In yet another embodiment, the hinge region comprises an N-terminal truncated form of IgG4 and/or a C-terminal truncated form of IgG4. Depending on the embodiment, these truncations can be at least about 50%, at least about 60%, at least about 70%, at least about 80%, or at least about 90% homologous to the hinge encoded by SEQ ID NO. 104.

In several embodiments, the chimeric receptor constructs employ a 2B4 intracellular signaling domain. In several embodiments, this domain is encoded by the amino acid sequence of SEQ ID NO. 35. In some embodiments, the 2B4 domain is encoded by the nucleic acid sequence of SEQ ID NO. 36. In some embodiments, the sequence of the 2B4 intracellular domain used in a chimeric receptor may vary from SEQ ID NO. 36, but remains, depending on the embodiment, at least 70%, at least 75%, at least 80%, at least 85%, at least 90%, or at least 95% homologous with SEQ ID NO. 36. In several embodiments, while the signaling domain of the chimeric receptor may vary from SEQ ID NO. 36, the chimeric receptor retains, or in some embodiments, has enhanced, NK cell activating and/or cytotoxic function. Likewise, in several embodiments an NKp80 intracellular domain is used, in several embodiments. In some embodiments, the NKp80 domain is the sole intracellular signaling domain, while in some embodiments, that domain is used in conjunction with one or more additional domains. In several embodiments, the NKp80 is encoded by the amino acid sequence of SEQ ID NO. 37. In some embodiments, the NKp80 domain is encoded by the nucleic acid sequence of SEQ ID NO. 38. In some embodiments, the sequence of the NKp80 intracellular domain used in a chimeric receptor may vary from SEQ ID NO. 38, but remains, depending on the embodiment, at least 70%, at least 75%, at least 80%, at least 85%, at least 90%, or at least 95% homologous with SEQ ID NO. 38. In several embodiments, while the signaling domain of the chimeric receptor may vary from SEQ ID NO. 38, the chimeric receptor retains, or in some embodiments, has enhanced, NK cell activating and/or cytotoxic function.

In several embodiments, the chimeric receptor uses a portion of a beta-adrenergic receptor as a transmembrane domain. In several embodiments, the portion comprises a portion of the beta-adrenergic extracellular domain. In several embodiments, the portion is a portion of the beta-adrenergic receptor transmembrane domain. In several embodiments, a combination of an extracellular domain and a transmembrane domain of the beta adrenergic receptor is used. Depending on the embodiment the portions are from the beta-1 and/or beta-2 adrenergic receptor. In several embodiments, a portion of the N-terminal extracellular region of the beta-2 adrenergic receptor is used. In several embodiments that portion has the amino acid sequence of SEQ ID NO. 39. In some embodiments, the extracellular beta-2 adrenergic domain is encoded by the nucleic acid sequence of SEQ ID NO. 40. In some embodiments, the sequence of the extracellular beta-2 adrenergic domain used in a chimeric receptor may vary from SEQ ID NO. 39, but remains, depending on the embodiment, at least 70%, at least 75%, at least 80%, at least 85%, at least 90%, or at least 95% homologous with SEQ ID NO. 39. In several embodiments, the first transmembrane helix of the beta-2 adrenergic receptor is used, optionally in conjunction with the extracellular beta-2 adrenergic domain. In several embodiments, the first transmembrane helix of the beta-2 adrenergic receptor has the amino acid sequence of SEQ ID NO. 41. In some embodiments, the first transmembrane helix of the beta-2 adrenergic receptor is encoded by the nucleic acid sequence of SEQ ID NO. 42. In some embodiments, the sequence of the first transmembrane helix of the beta-2 adrenergic receptor used in a chimeric receptor may vary from SEQ ID NO. 41, but remains, depending on the embodiment, at least 70%, at least 75%, at least 80%, at least 85%, at least 90%, or at least 95% homologous with SEQ ID NO. 41.

In one embodiment, the chimeric receptor comprises CD8, truncated NKG2D, CD8α, transmembrane domain, a CD16 intracellular domain, and 4-1BB as a costimulatory molecule. In several embodiments, such a construct is encoded by SEQ ID NO. 25. In some embodiments, the chimeric receptor may vary from SEQ ID NO. 25, but remains, depending on the embodiment, at least 70%, at least 75%, at least 80%, at least 85%, at least 90%, or at least 95% homologous with SEQ ID NO. 25. In several embodiments, hinge regions surrounding CD8 are increased by way of addition of GS linkers (disclosed herein), such as GS3, by way of non-limiting example. In such embodiments, the construct is encoded by the nucleic acid of SEQ ID NO. 43. In some embodiments, the chimeric receptor may vary from SEQ ID NO. 43, but remains, depending on the embodiment, at least 70%, at least 75%, at least 80%, at least 85%, at least 90%, or at least 95% homologous with SEQ ID NO. 43. In several embodiments, hinge regions surrounding CD8 are increased by way of addition of longer GS linkers, such as GS12, or other linker. In several embodiments, hinge regions are decreased by way of truncating CD8. For example, in several embodiments, the N-terminal region of CD8α is truncated by at least 20%, at least 30%, at least 40%, or at least 50%. In several embodiments, the CD8 hinge is replaced with a GS linker. For example, in several embodiments, the hinge region comprises a GS3 linker, thereby the construct comprises NKG2D-GS3-CD16-4-1BB. In one embodiment, such a construct is encoded by the nucleic acid of SEQ ID NO. 44. In some embodiments, the chimeric receptor may vary from SEQ ID NO. 44, but remains, depending on the embodiment, at least 70%, at least 75%, at least 80%, at least 85%, at least 90%, or at least 95% homologous with SEQ ID NO. 44. In several embodiments, neither CD8 nor GSn are used. In one embodiment, such a construct is encoded by the nucleic acid of SEQ ID NO. 45. In some embodiments, the chimeric receptor may vary from SEQ ID NO. 45, but remains, depending on the embodiment, at least 70%, at least 75%, at least 80%, at least 85%, at least 90%, or at least 95% homologous with SEQ ID NO. 45.

As discussed above, in several embodiments, codon optimized sequences are employed. For example in several embodiments, codon optimization (full or partial) is performed on the NKG2D domain of a chimeric receptor. In several embodiments, however, codon optimization is not performed. In several embodiments, a chimeric receptor construct is provided with an NKG2D extracellular domain that is not optimized, a CD8α hinge, and a 4-1BB signaling domain. In several embodiments, a chimeric receptor construct is provided with an NKG2D extracellular domain that is not optimized, a CD8α hinge and transmembrane domain, and a 4-1BB signaling domain. In several embodiments, a chimeric receptor construct is provided with an NKG2D extracellular domain that is not optimized, a CD8α hinge and transmembrane domain, a 4-1BB signaling domain and a 2B4 signaling domain. In several embodiments, such a construct has the nucleic acid sequence of SEQ ID NO. 46. In some embodiments, the chimeric receptor may vary from SEQ ID NO. 46, but remains, depending on the embodiment, at least 70%, at least 75%, at least 80%, at least 85%, at least 90%, or at least 95% homologous with SEQ ID NO. 46.

In several embodiments, a chimeric receptor construct is provided with an NKG2D extracellular domain that is not optimized, a beta-adrenergic derived transmembrane domain, and a 4-1BB signaling domain. In several embodiments, a chimeric receptor construct is provided with an NKG2D extracellular domain that is not optimized, a beta-adrenergic derived transmembrane domain made up of the extracellular region of the beta-2 adrenergic receptor and the first transmembrane helix of the beta-2 adrenergic receptor, and a 4-1BB signaling domain. In several embodiments, a chimeric receptor construct is provided with an NKG2D extracellular domain that is not optimized, a beta-adrenergic derived transmembrane domain made up of the extracellular region of the beta-2 adrenergic receptor and the first transmembrane helix of the beta-2 adrenergic receptor, a 4-1BB signaling domain and a 2B4 signaling domain. In several embodiments, such a construct has the nucleic acid sequence of SEQ ID NO. 47. In some embodiments, the chimeric receptor may vary from SEQ ID NO. 47, but remains, depending on the embodiment, at least 70%, at least 75%, at least 80%, at least 85%, at least 90%, or at least 95% homologous with SEQ ID NO. 47.

In several embodiments, a chimeric receptor construct is provided with an NKG2D extracellular domain that is not optimized, a CD8α hinge, and a 2B4 signaling domain. In several embodiments, a chimeric receptor construct is provided with an NKG2D extracellular domain that is not optimized, a CD8α hinge and transmembrane domain, and both a 2B4 and a 4-1BB signaling domain. In several embodiments, a chimeric receptor construct is provided with an NKG2D extracellular domain that is not optimized, a CD8α hinge and transmembrane domain, a 4-1BB signaling domain and a 2B4 signaling domain, as well as a NKp80 domain. In several embodiments, a GS linker, such as a GS3 linker joins the 2B4 and NKp80 domains. In several embodiments, such a construct has the nucleic acid sequence of SEQ ID NO. 48. In some embodiments, the chimeric receptor may vary from SEQ ID NO. 48, but remains, depending on the embodiment, at least 70%, at least 75%, at least 80%, at least 85%, at least 90%, or at least 95% homologous with SEQ ID NO. 48.

In several embodiments, a chimeric receptor construct is provided with an NKG2D extracellular domain that is not optimized, a CD8α hinge, and a NKp80 signaling domain. In several embodiments, a chimeric receptor construct is provided with an NKG2D extracellular domain that is not optimized, a CD8α hinge and transmembrane domain, and a NKp80 signaling domain. In several embodiments, a chimeric receptor construct is provided with an NKG2D extracellular domain that is not optimized, a CD8α hinge and transmembrane domain, a 4-1BB signaling domain and a NKp80 domain. In several embodiments, a GS linker, such as a GS3 linker joins the 4-1BB and NKp80 domains. In several embodiments, such a construct has the nucleic acid sequence of SEQ ID NO. 49. In some embodiments, the chimeric receptor may vary from SEQ ID NO. 49, but remains, depending on the embodiment, at least 70%, at least 75%, at least 80%, at least 85%, at least 90%, or at least 95% homologous with SEQ ID NO. 49.

In several embodiments, a CD8 transmembrane domain is coupled with a 2B4 intracellular domain. In several embodiments, a CD8 transmembrane domain is replaced with a 2B4 domain that is transmembrane and intracellular. In several embodiments, the CD8 transmembrane domain is replaced with 2B4 and 4-1BB is expressed in a proximal configuration.

In several embodiments, a CD16 intracellular signaling domain is coupled with a CD3zeta or gamma subunit which are exogenously expressed in trans to the chimeric receptors described herein. As discussed above, such constructs can result in unexpectedly enhanced signal transduction, and thus an unexpected increase in cytotoxic effects of the NK cells.

In several embodiments, the chimeric receptors are configured to dimerize, as discussed in additional detail herein. In several embodiments a truncated NKG2D receptor according to several embodiments disclosed herein is optionally dimerized. Dimerization may comprise homodimers or heterodimers, depending on the embodiment. In several embodiments, dimerization results in a shift of avidity of the chimeric receptor (and hence the NK cells expressing the receptor) to better ligand recognition with a coordinate balance in reduced (or lack) of adverse toxic effects. In still further embodiments, the extracellular receptor domain further comprises a CD8α signal peptide. In several embodiments, the chimeric receptors employ internal dimers, or repeats of one or more component subunits. For example, in several embodiments, the chimeric receptor comprises a NKG2D extracellular domain coupled to a second NKG2D extracellular domain, and a transmembrane/signaling region (or a separate transmembrane region along with a separate signaling region). In several embodiments, one or more of the NKG2D extracellular domains are codon optimized. In several embodiments, the two NKG2D extracellular domains are separated by a linker, for example a GSn linker. In one embodiment, a GS3 linker is used. In several embodiments, the transmembrane domain comprises an extracellular region of the beta-adrenergic receptor. In several embodiments, the transmembrane domain transmembrane domain comprises an extracellular region of the beta-2 adrenergic receptor and further comprises the first transmembrane domain of the beta-2 adrenergic receptor. In several embodiments, the signaling region comprises 4-1BB. In several embodiments, the signaling region comprises NKp80. In several embodiments, the signaling region comprises a CD16 transmembrane-intracellular domain. In several embodiments, the signaling region comprises 4-1BB in conjunction with NKp80 or a CD16 transmembrane-intracellular domain. In several embodiments, the chimeric receptor has the nucleic acid sequence of SEQ ID NO. 50. In some embodiments, the chimeric receptor may vary from SEQ ID NO. 50, but remains, depending on the embodiment, at least 70%, at least 75%, at least 80%, at least 85%, at least 90%, or at least 95% homologous with SEQ ID NO. 50. In several embodiments, the chimeric receptor has the nucleic acid sequence of SEQ ID NO. 51. In some embodiments, the chimeric receptor may vary from SEQ ID NO. 51, but remains, depending on the embodiment, at least 70%, at least 75%, at least 80%, at least 85%, at least 90%, or at least 95% homologous with SEQ ID NO. 51. In several embodiments, the chimeric receptor has the nucleic acid sequence of SEQ ID NO. 52. In some embodiments, the chimeric receptor may vary from SEQ ID NO. 52, but remains, depending on the embodiment, at least 70%, at least 75%, at least 80%, at least 85%, at least 90%, or at least 95% homologous with SEQ ID NO. 52. In several embodiments, the chimeric receptor comprises a hinge region. In several embodiments, CD8α is repurposed to serve as a hinge region (encoded, in several embodiments, by the nucleic acid sequence of SEQ ID NO: 5). In several embodiments, the chimeric receptor comprises a CD8α transmembrane domain. In several embodiments, the signaling region comprises 4-1BB in conjunction with 2B4 and CD3zeta. In some embodiments, the chimeric receptor comprises the fragment of NKG2D that is codon optimized coupled to a GS3 linker, an additional NKG2D fragment, a CD8α hinge, a CD8α transmembrane domain, and an effector domain comprising 4-1BB and CD3zeta. In several embodiments, the chimeric receptor has the nucleic acid sequence of SEQ ID NO. 66. In some embodiments, the chimeric receptor may vary from SEQ ID NO. 66, but remains, depending on the embodiment, at least 70%, at least 75%, at least 80%, at least 85%, at least 90%, or at least 95% homologous with SEQ ID NO. 50. In several embodiments, the chimeric receptor chimeric receptor comprises the amino acid sequence of SEQ ID NO: 67. In some embodiments, the chimeric receptor may vary from SEQ ID NO. 66, but remains, depending on the embodiment, at least 70%, at least 75%, at least 80%, at least 85%, at least 90%, or at least 95% homologous with SEQ ID NO. 50.

In several embodiments, the chimeric receptors are configured to be bispecific, as discussed in additional detail herein. In several embodiments, a truncated NKG2D receptor according to several embodiments disclosed herein is bispecific due to a second peptide that binds, for example, non-NKG2D ligands. In several embodiments, bi-specificity results in a shift of the targeting of the chimeric receptor (and hence the NK cells expressing the receptor) to better target cell recognition with a coordinate balance in reduced (or lack) of adverse toxic effects. In still further embodiments, the extracellular receptor domain further comprises a CD8α signal peptide. For example, in several embodiments, the chimeric receptor comprises a NKG2D extracellular domain coupled to a second extracellular domain that binds other (non-NKG2D) ligands, and a transmembrane/signaling region (or a separate transmembrane region along with a separate signaling region). In several embodiments, the two extracellular domains are separated by a linker, for example a GSn linker. In one embodiment, a GS3 linker is used.

According to several embodiments disclosed herein, additional chimeric receptors employing codon optimized NKG2D domains are provided for (optionally, these constructs can also be replicated with non-optimized or partially optimized domains). For example, in several embodiments, a codon optimized extracellular domain is coupled with a hinge and at least two transmembrane/signaling domains. In several embodiments, the multiple signaling domains provide enhanced cytotoxic efficacy of the NK cells because multiple, non-redundant signal cascades are set in motion. While in some embodiments these multiple pathways may converge on a single signaling molecule (e.g., IFNγ), the overall cytotoxic effect is unexpectedly increased because of the overall magnitude of signaling molecules driving a cytotoxic endpoint. As a non-limiting example, in several embodiments an NKG2D is coupled to a CD8α hinge followed by a CD16 transmembrane-intracellular signaling domain and a 4-1BB signaling domain. In several embodiments, this construct further comprises a 2B4 signaling domains. In several embodiments, such a chimeric receptor has the nucleic acid sequence of SEQ ID NO. 53. In some embodiments, the chimeric receptor may vary from SEQ ID NO. 53, but remains, depending on the embodiment, at least 70%, at least 75%, at least 80%, at least 85%, at least 90%, or at least 95% homologous with SEQ ID NO. 53. In additional embodiments, the NKG2D-CD8α-CD16IC/TM construct further comprises a NKp80 signaling domain. In several embodiments, such a construct further comprises a GS3 linker between the 4-1BB and NKp80 domains. In several embodiments, such a chimeric receptor has the nucleic acid sequence of SEQ ID NO. 54. In some embodiments, the chimeric receptor may vary from SEQ ID NO. 54, but remains, depending on the embodiment, at least 70%, at least 75%, at least 80%, at least 85%, at least 90%, or at least 95% homologous with SEQ ID NO. 54.

In still additional embodiments, certain components of a chimeric receptor can be replaced with one or more additional subunits that lead to enhanced efficacy (e.g., activation or cytotoxicity of NK cells). For example, in one embodiment, a CD16 intracellular signaling domain can be replaced with a quad-repeat of DAP10 (e.g., 4×DAP10). In an additional embodiment, a CD16 intracellular signaling domain can be replaced with a Zap70 subunit. Certain such embodiments lead to unexpectedly enhanced NK cell cytotoxicity.

In several additional embodiments, the effector domain comprises one or more consensus hemi-ITAM sequences to enhance the transduction of activation signaling upon ligand binding. In additional embodiments, the inclusion of a GS linker between the signaling domains of 4-1BB, CD16, NCR1, NCR2 and/or NCR3 enhances signal transduction. Moreover, in several embodiments one or both of CD3zeta and FcRγ are additionally expressed along with the chimeric receptors described herein (either on the same or a separate construct), which results in unexpectedly enhanced signal transduction, and thus an unexpected increase in cytotoxic effects of the NK cells. Depending on the embodiment, the engineered expression of one or more of CD3zeta and FcRγ supplements endogenous expression of these molecules by NK cells, thereby further enhancing the signaling and ultimate cytotoxic potency of the NK cells.

Optionally, depending on the embodiment, any of the polynucleotides disclosed herein may also encode truncations and/or variants of one or more of the constituent subunits of a chimeric receptor, yet retain their ability to direct NK cells to target cells and in several embodiments unexpectedly enhance cytotoxicity upon binding. In addition, any of the polynucleotides disclosed herein may also optionally include codon-optimized nucleotide sequences encoding the various constituent subunits of a chimeric receptor. As used herein, the terms “fragment” and “truncated” shall be given their ordinary meaning and shall also include N- and C-terminal deletion variants of proteins.

The polynucleotides encoding the chimeric receptors described herein may be inserted into vectors to achieve recombinant protein expression in NK cells. In one embodiment, the polynucleotide is operably linked to at least one regulatory element for the expression of the chimeric receptor. In specific embodiments, transcriptional regulatory elements heterologous, such as, for example an internal ribosome entry site (IRES) or enhancer element, to the peptides disclosed herein are employed to direct the transcription of the chimeric receptor. In some embodiments, the polynucleotide comprises one or more cytosolic protease cleavage sites. In some embodiments, the cleavage site is recognized and cleaved by a cytosolic protease. In some embodiments, this cleavage site is selected from the group comprising a T2A cleavage site, a P2A cleavage site, an E2A cleavage site, and an F2A cleavage site. Depending on the embodiment, the various constituent parts of a chimeric receptor can be delivered to an NK cell in a single vector, or alternatively in multiple vectors. In some embodiments, a chimeric receptor construct is delivered in a single vector, while another factor that enhances efficacy of the chimeric receptor, such as mbIL15, is delivered in a separate vector. In several embodiments, a chimeric receptor and a factor that enhances efficacy of the chimeric receptor (e.g., mbIL15), is delivered in a single vector. Regardless of the number of vectors used, any polynucleotide may optionally include a tag sequence, allowing identification of the presence of NK cells expressing the construct. For example, in several embodiments a FLAG tag (DYKDDDDK, SEQ ID NO. 55) is used. Also available are other tag sequences, such as a polyhistidine tag (His-tag) (HHHHHH, SEQ ID NO. 56), HA-tag or myc-tag (EQKLISEEDL; SEQ ID NO: 57). Alternatively, green fluorescent protein, or other fluorescent moiety, is used. Combinations of tag types can also be used, to individually recognize sub-components of a chimeric receptor.

In several embodiments, the polynucleotide encoding the chimeric receptor is an mRNA that may be introduced into NK cells by electroporation. In another embodiment, the vector is a virus, preferably a retrovirus, which may be introduced into NK cells by transduction. In several embodiments, the vector is a Murine Stem Cell Virus (MSCV). In additional embodiments, other vectors may be used, for example lentivirus, adenovirus, adeno-associated virus, and the like may be used. In several embodiments, non-HIV-derived retroviruses are used. The vector chosen will depend upon a variety of factors, including, without limitation, the strength of the transcriptional regulatory elements and the cell to be used to express a protein. The vector can be a plasmid, phagemid, cosmid, viral vector, phage, artificial chromosome, and the like. In additional embodiments, the vectors can be episomal, non-homologously, or homologously integrating vectors, which can be introduced into the appropriate cells by any suitable means (transformation, transfection, conjugation, protoplast fusion, electroporation, calcium phosphate-precipitation, direct microinjection, etc.) to transform them. Other approaches to induce expression of chimeric receptors in NK cells are used in several embodiments, including for example, the SV40 early promoter region, the promoter contained in the 3′ long terminal repeat of Rous sarcoma virus, the herpes thymidine kinase promoter, the regulatory sequences of the metallothionein gene, an adenovirus (ADV) promoter, a cytomegalovirus (CMV) promoter, the bovine papilloma virus (BPV) promoter, the parovirus B19p6 promoter, the beta-lactamase promoter, the tac promoter, the nopaline synthetase promoter region or the cauliflower mosaic virus 35S RNA promoter, the promoter of ribulose biphosphate carboxylase, the Gal 4 promoter, the ADC (alcohol dehydrogenase) promoter, the PGK (phosphoglycerol kinase) promoter, the synthetic MND promoter containing the U3 region of a modified MoMuLV LTR with the myeloproliferative sarcoma virus enhancer, and the alkaline phosphatase promoter.

Natural killer cells may be engineered to express the chimeric receptors disclosed herein. Chimeric receptor expression constructs may be introduced into NK cells using any of the techniques known to one of skill in the art. In one embodiment, the chimeric receptors are transiently expressed in the NK cells. In another embodiment, the chimeric receptors are stably expressed in NK cells. In an additional embodiment, the NK cells are autologous cells. In yet another embodiment, the NK cells are donor-derived (allogeneic) cells.

Further provided herein are methods of treating a subject having cancer or an infectious disease comprising administering to the subject a composition comprising NK cells engineered to express a chimeric receptor as disclosed herein, the chimeric receptor designed to target a marker or ligand expressed differentially on the damaged or diseased cells or tissue (e.g., expressed to a different degree as compared to a normal cell or tissue). As used herein, the terms “express”, “expressed” and “expression” be given their ordinary meaning and shall refer to allowing or causing the information in a gene or polynucleotide sequence to become manifest, for example producing a protein by activating the cellular functions involved in transcription and translation of a corresponding gene or DNA sequence. The expression product itself, e.g., the resulting protein, may also be said to be “expressed” by the cell. An expression product may be characterized as intracellular, extracellular or transmembrane. The term “intracellular” shall be given its ordinary meaning and shall refer to inside a cell. The term “extracellular” shall be given its ordinary meaning and shall refer to outside a cell. The term “transmembrane” shall be given its ordinary meaning and shall refer to at least a portion of a polypeptide is embedded in a cell membrane. The term “cytoplasmic” shall be given its ordinary meaning and shall refer to residing within the cell membrane, outside the nucleus. As used herein, the terms “treat,” “treating,” and “treatment” in the context of the administration of a therapy to a subject shall be given their ordinary meaning and shall refer to the beneficial effects that a subject derives from a therapy. In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Administration can be by a variety of routes, including, without limitation, intravenous, intra-arterial, subcutaneous, intramuscular, intrahepatic, intraperitoneal and/or local delivery to an affected tissue. Doses of NK cells can be readily determined for a given subject based on their body mass, disease type and state, and desired aggressiveness of treatment, but range, depending on the embodiments, from about 10⁵ cells per kg to about 10¹² cells per kg (e.g., 10⁵⁻, 10⁷, 10⁷⁻, 10¹⁰, 10¹⁰⁻, 10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated. Various embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

Further, various embodiments provided for herein include treatment or prevention of the following non-limiting examples of infectious diseases including, but not limited to, infections of bacterial origin may include, for example, infections with bacteria from one or more of the following genera: Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia, and mutants or combinations thereof. In several embodiments, methods are provided to treat a variety to treat viral infections, such as those caused by one or more viruses, such as adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus.

In some embodiments, also provided herein are nucleic acid and amino acid sequences that have homology of at least 80%, 85%, 90%, 95%, 96%, 97%, 98%, 99% (and ranges therein) as compared with the respective nucleic acid or amino acid sequences of SEQ ID NOS. 1-68 and that also exhibit one or more of the functions as compared with the respective SEQ ID NOS. 1-68: including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

There are provided for herein, according to several embodiments, polynucleotides encoding chimeric receptors, comprising an extracellular receptor domain, wherein the extracellular receptor domain comprises a peptide that binds native ligands of Natural Killer Group 2 member D (NKG2D), wherein the peptide that binds native ligands of NKG2D is a fragment of NKG2D, an effector domain comprising a transmembrane region and an intracellular signaling domain. In several embodiments, the fragment of NKG2D is encoded by a polynucleotide comprising the sequence of SEQ ID NO. 2 or 3 or 68, or functional equivalent thereof. In several embodiments, the polynucleotide encodes an effector domain comprising CD16. In several embodiments, the polynucleotide encodes an effector domain comprising NCR1. In several embodiments, the polynucleotide encodes an effector domain comprising NCR2. In several embodiments, the polynucleotide encodes an effector domain comprising NCR3. In some embodiments, the polynucleotide encodes an additional effector domain portion comprising 4-1BB. In several embodiments, the polynucleotide encodes a chimeric receptor made up of NKG2D and CD16. In several embodiments, the polynucleotide encodes a chimeric receptor made up of NKG2D and NCR1. In several embodiments, the polynucleotide encodes a chimeric receptor made up of NKG2D and NCR2. In additional embodiments, the polynucleotide encodes a chimeric receptor made up of NKG2D coupled to CD16 and optionally 4-1BB. In several embodiments, CD16 is replaced by NCR1, and in some embodiments, by NCR2, or even NCR3, depending on the embodiment. In several embodiments, the effector domain further comprises a GS linker between, for example, 4-1BB and one of CD16, NCR1, NCR2, or NCR3.

In several embodiments, the extracellular receptor domain further comprises a hinge region. In several embodiments, the hinge region comprises CD8α. However, in additional embodiments, the hinge region further comprises one or more linkers, which in some embodiments, comprise GS9, CD8α/GS3, truncated CD8α, GS3, and the like.

In several embodiments, the extracellular receptor domain further comprises a CD8α signal peptide. In several embodiments, the effector domain comprises one or more hemi-ITAM sequences. In several embodiments, the chimeric receptor does not comprise DNAX-activating protein 10 (DAP10). In several embodiments, the chimeric receptor does not comprise an ITAM motif, but rather employs an alternative signaling region, such as an ITSM, hemi-tam or other co-stimulatory region.

In one embodiment, there is provided a polynucleotide encoding a chimeric receptor comprising an extracellular receptor domain, wherein the extracellular receptor domain comprises a peptide that binds native ligands of Natural Killer Group 2 member D (NKG2D), wherein the peptide that binds native ligands of NKG2D is a fragment of NKG2D, a transmembrane region, wherein the transmembrane region comprises CD8α, and an effector domain, wherein the effector domain comprises 4-1BB and CD3 zeta, wherein the polynucleotide is co-expressed with an additional construct encoding membrane-bound interleukin 15 (mbIL15).

There is also provided in several embodiments, a polynucleotide encoding a chimeric receptor comprising an extracellular receptor domain, wherein the extracellular receptor domain comprises a peptide that binds native ligands of Natural Killer Group 2 member D (NKG2D), wherein the peptide that binds native ligands of NKG2D is a fragment of NKG2D, a transmembrane region, wherein the transmembrane region comprises CD8α, and an effector domain, wherein the effector domain comprises 4-1BB and the intracellular domain of 2B4 or DAP10. The polynucleotide encoding a chimeric receptor as described herein comprises a second peptide that binds native ligands of NKG2D. In several embodiments, the native ligands of NKG2D include, but are not limited to, MICA, MICB, ULBP1, ULBP2, ULBP3, ULBP4, ULBP5 or ULBP6. In several embodiments, the portion of the chimeric receptor that binds native ligands of NKG2D has at least 80% homology to SEQ ID NO: 1, 2, 3, or 68.

In several embodiments, the provided polynucleotide is an mRNA. In some embodiments, the polynucleotide is operably linked to at least one regulatory element for the expression of the chimeric receptor. As used herein, the terms “nucleic acid,” “nucleotide,” and “polynucleotide” shall be given their ordinary meanings and shall include deoxyribonucleotides, deoxyribonucleic acids, ribonucleotides, and ribonucleic acids, and polymeric forms thereof, and includes either single- or double-stranded forms. Nucleic acids include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleic acid analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), locked-nucleic acids (LNAs), and the like. As used herein, the term “operably linked,” for example in the context of a regulatory nucleic acid sequence being “operably linked” to a heterologous nucleic acid sequence, shall be given its ordinary meaning and shall mean that the regulatory nucleic acid sequence is placed into a functional relationship with the heterologous nucleic acid sequence. In the context of an IRES, “operably linked to” refers to a functional linkage between a nucleic acid sequence containing an internal ribosome entry site and a heterologous coding sequence initiation in the middle of an mRNA sequence resulting in translation of the heterologous coding sequence. As used herein, the term “vector” shall be given its ordinary meaning and shall refer to a vehicle by which a DNA or RNA sequence (e.g., a foreign gene) can be introduced into a genetically engineered cell, so as to transform the genetically engineered cell and promote expression (e.g., transcription and/or translation) of the introduced sequence. Vectors include viruses, plasmids, phages, etc. The term “chimeric receptor” as used herein shall be given its ordinary meaning and shall refer to a cell-surface receptor comprising at least two polypeptide domains not naturally found together on a single protein. The term “chimeric receptor complex” as used herein refers to a first polypeptide, which may comprise at least two polypeptide domains in a combination that are not naturally found together on a single protein, which first polypeptide is associated with a second polypeptide, for example, an adaptor polypeptide, a signaling molecule, or a stimulatory molecule. Additional terms relating to generation and use of chimeric receptors as disclosed here are readily understood by one of ordinary skill in the art and can also be found in International Publication WO 2014/117121 and U.S. Pat. No. 7,994,298, each of which are incorporated by reference in their entirety herein.

Additionally provided, according to several embodiments, is a vector comprising the polynucleotide encoding any of the polynucleotides provided for herein, wherein the polynucleotides are optionally operatively linked to at least one regulatory element for expression of a chimeric receptor. In several embodiments, the vector is a retrovirus.

Further provided herein are engineered natural killer cells comprising the polynucleotide, vector, or chimeric receptors as disclosed herein. In several embodiments, these NK cells are suitable for use in the treatment of prevention of disease, such as, for example, cancer and/or infectious disease.

### EXAMPLES

### Methods

The following experimental methods and materials were used in the non-limiting experimental examples disclosed below.

### Cell Lines and Culture Conditions

The human acute lymphoblastic leukemia cell line REH, human osteosarcoma cell line U-2 OS and human embryonic kidney fibroblast 293T (HEK 293T) cells were obtained from the American Type Culture Collection (ATCC; Manassas, Virginia). REH cells were maintained and grown in Roswell Park Memorial Institute series 1640 (RPMI-1640; Gibco, Carlsbad, California) supplemented with 10% fetal bovine serum (FBS; Hyclone, Logan, Utah) and 1% penicillin-streptomycin. Both HEK 293T and U-2 OS cells were maintained and grown in Dulbecco's modified Eagles Medium (DMEM; Hyclone) supplemented with 10% FBS and 1% penicillinstreptomycin. All mammalian cells were incubated at 37° C. with 5% CO2.

### DNA Plasmids

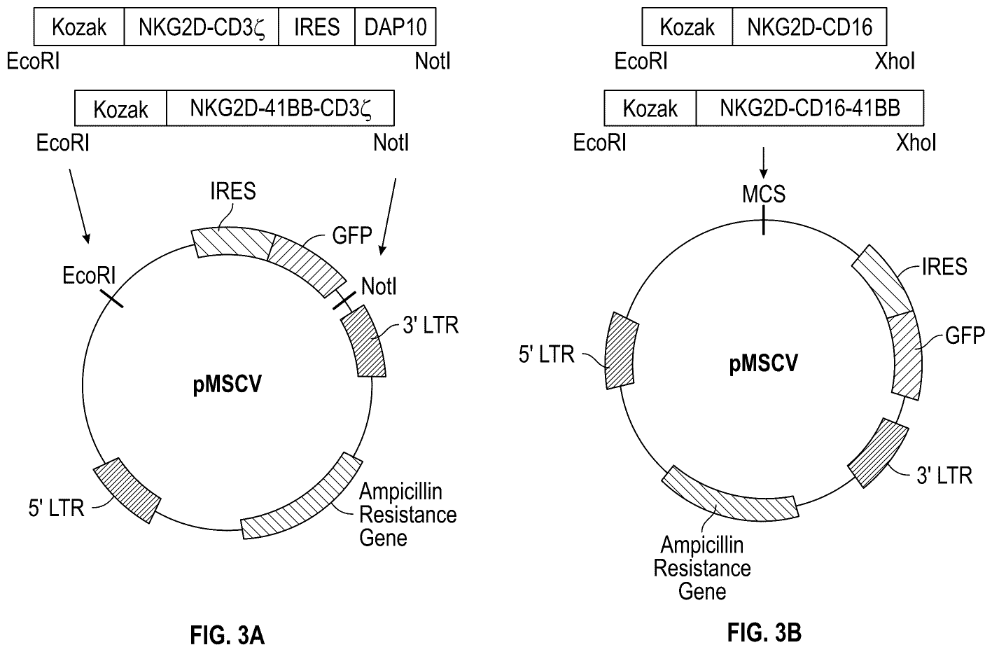

A DNA plasmid containing the chimeric receptor NKG2D-DAP10-CD3zeta was made as previously described (see Chang et al. Cancer Research, Vol. 73(6): 2013). Splicing by overlapping extension polymerase chain reaction (SOE-PCR) was used to fuse the individual domains forming the NKG2D-41BB-CD3zeta construct. That construct was then inserted into the Murine Stem Cell Virus (MSCV) retroviral vector (FIG. 3A). The constructs for NKG2D-CD16 and NKG2D-CD16-41BB were codon optimized and inserted into the MSCV vector (FIG. 3B) by GenScript (Nanjing, China). The sequences of the constructs were verified by DNA sequencing.

### Expansion of Human NK Cells

Human peripheral blood mononuclear cells (PBMCs) were obtained by Ficoll density centrifugation of blood samples from healthy adult donors. To expand the NK cells, PBMCs were cultured with K562 genetically modified with membrane bound IL-15 and 4-1BB ligand (K562-mb15-41BBL). Cells were cultured in Stem Cell Growth Medium (SCGM; Cell Genix, Freiburg, Germany) supplemented with 40IU of IL-2/ml every two days.

After 7 days of culture, NK cells were T-cell depleted using anti-CD3 Dynabeads (Invitrogen, Carlsbad, California). NK cells were then cultured in SCGM supplemented with 40-200 IU of IL-2/ml every two days.

### Production of Retrovirus and Transduction of NK Cells

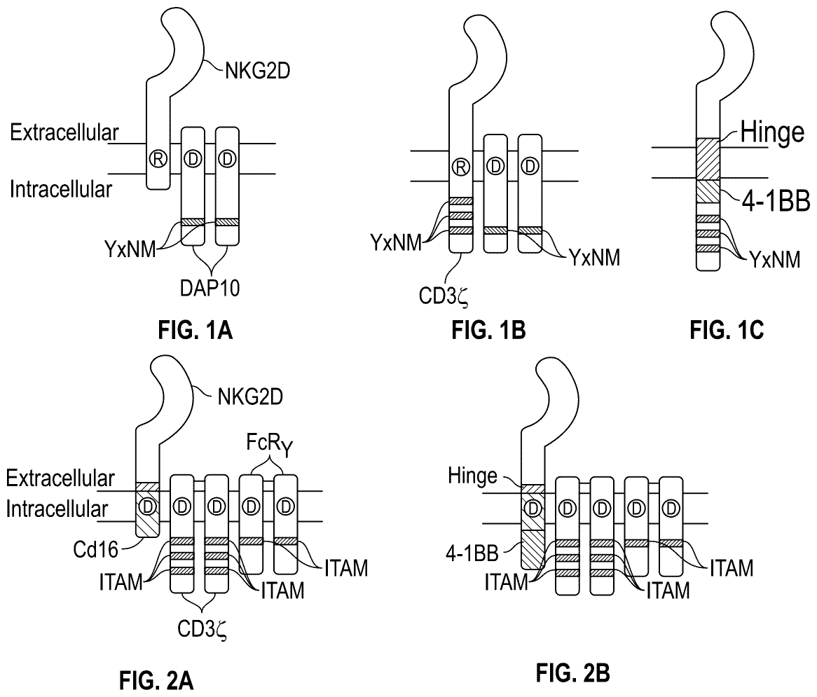

Production of retrovirus was carried out by transiently transfecting HEK 293T cells with retroviral packaging plasmids. HEK 293T cells were first seeded to a concentration of 2.5×10⁶ cells in 12 ml of DMEM 18 hours before the transfection. The cells were then transfected with 3.5 μg of MSCV vector containing the respective NKG2D chimeric receptors (non-limiting constructs are illustrated schematically in FIGS. 1B-1C and 2A-2B), 3.5 μg of pEQ-PAM3, and 3.0 μg of pRDF. For control, empty MSCV vector containing GFP was used. X-tremeGENE 9 DNA Transfection Reagent (Roche, Basel, Switzerland) was used for the transfection. DMEM was replaced with conditioned RPMI-1640 24 hours after the transfection.

Transduction of NKG2D chimeric receptor transgene into NK cells was done 18 hours after the changing of media. NK cells were first suspended at a concentration of 0.25×10⁶ cells in 2 ml of conditioned RPMI-1640. Cells were subsequently seeded into RetroNectin (TaKaRa, Otsu, Japan) coated tubes. RPMI-1640 containing the retrovirus (virus supernatant) was harvested from the HEK 293T cell cultures and fresh conditioned medium was added back to the cultures. The viral supernatant was supplemented with 200 IU of IL-2/ml and 3 ml of the viral supernatant was dispensed into each RetroNectin coated tubes (containing the seeded NK cells). In accordance with certain embodiments of producing NK cells, seeded NK cells were transduced six times, once every 12 hours with fresh viral media. Transduced NK cells were then harvested 48 hours after the last transduction, and cultured in SCGM with the addition of 200 IU of IL-2/ml every two days. The transduced NK cells were used for experiments 14 to 28 days after expansion.

### Detection of Chimeric Receptor Expression by Flow Cytometry

Transduced NK cells were washed once with phosphate-buffered saline containing albumin, and 2 μl of rabbit serum was added. The cells were then stained with peridinin chlorophyll (PerCP)-conjugated anti-human NKG2D antibody (clone 149810; R&D Systems, Minneapolis, USA) for 10 minutes in the dark. For controls, the transduced NK cells were stained with the respective PerCP-conjugated IgG isotype antibody. All NK cells were washed again and fixed with 300 μl 0.5% formaldehyde before analysis using Accuri C6 flow cytometer (BD, Franklin Lakes, New Jersey). Data was analyzed using a paired t-test.

### Cytotoxicity Assays

REH cells were stained with calcein AM red-orange (Thermo Fisher Scientific, Waltham, Massachusetts). REH cells were seeded into a 96-well round bottom plate (CoStar, Corning, New York). Transduced NK cells were then added at various effector:target (E:T) ratio. The cell cultures were incubated for four hours at 37° C. and 5% CO2. Stained viable target cells were counted using the Accuri C6 flow cytometer. U-2 OS cells were seeded into 96-well flat bottom white plate (Costar) and incubated for four hours. Transduced NK cells was then added according to different E:T ratios. Cell cultures were then incubated for another four hours. Prior to analysis, Bright-Glo substrate (Promega, Madison, Wisconsin) was added to the cells. Intensity of luminescence from viable target cells was measured using FLx800 Fluorescence Reader (Bio Tek, Winooski, Vermont). Differences between intensity of luminescence and control were converted to percentage cytotoxicity.

### Interferon Gamma (IFNγ) Production Assay

To determine the amount of IFNγ produced by the NK cells, effector and target cells were first cultured with (E:T of 1:1) or without REH in a 96-well round bottom plate. Cells were incubated for one hour before the addition of GolgiPlug (brefeldin A; BD Biosciences). After another 5 hours of culture, cells were labeled with phycoerythrin (PE)-conjugated anti-human CD56 antibody (clone MY31, BD Biosciences). Cells were permeabilized using a proprietary permeabilization reagent and incubated for 40 minutes in the dark. The cells were then washed with a proprietary wash buffer. Intracellular IFNγ was detected with allophycocyanin (APC)-conjugated IFNγ antibody (clone 25723.11; BD Biosciences) for 45 minutes. The cells were then fixed and analyzed using Accuri C6 flow cytometer.

### Example 1—CD3-Zeta Containing NKG2D Constructs

As disclosed herein, various constructs comprising NKG2D and/or NKG2D variants coupled with various transmembrane and/or signaling domains are provided. The present experiment was conducted to evaluate the expression and cytotoxic activity of constructs comprising CD3zeta signaling domains. Two CD3zeta constructs were prepared and tested according to the methods and materials described above. Depending on the construct, the methods used can be readily adjusted to account for variations required for generating, expressing and testing a construct. The two constructs were NKG2D-DAP10-CD3zeta and NKG2D-41BB-CD3zeta. For reference FIG. 1A schematically depicts an endogenous NKG2D. In NK cells, ionic interactions between the transmembrane region of NKG2D allow association with its adaptor protein DAP10 (Wu et al., 1999). Upon ligand binding, NKG2D signals are transduced through the signaling motif, YxNM, found on DAP10. CD3zeta transduce signals through its immunoreceptor tyrosine-based activation motif (ITAM; Lanier, 2008). The two experimental constructs are illustrated schematically in FIGS. 1B and 1C, respectively. FIG. 1B shows NKG2D-DAP10-CD3zeta, with signaling occurring through both the YxNM and ITAM motifs. FIG. 1C shows the NKG2D-41BB-CD3zeta construct, which employs a CD8α hinge region as a transmembrane domain and 4-1BB and CD3zeta as signaling domains.

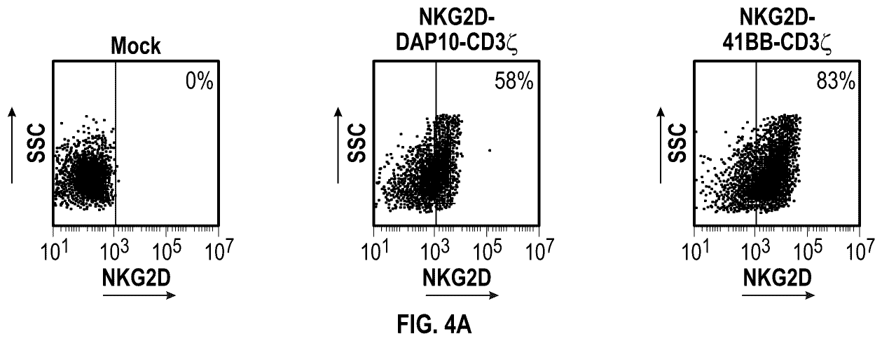

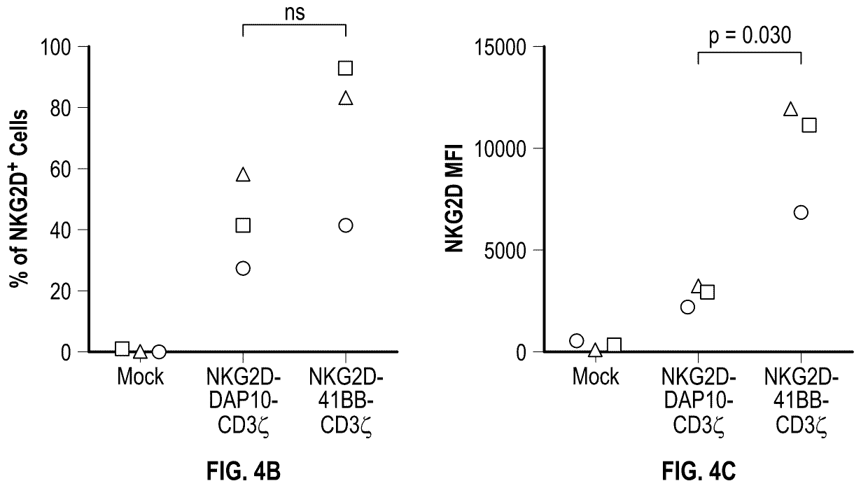

The ability of NK cells to effectively express these constructs was first assessed. NK cells expanded from PBMC of healthy adult donors were transduced with one of the two chimeric receptors. Mock-transduced NK cells were used as control (transduced with empty MSCV vector containing GFP only). The presence and relative abundance of the chimeric receptors were determined through staining the NK cells with a Per-CP conjugated anti-NKG2D antibody. FIG. 4A depicts representative flow cytometry data related to the percentage of NKG2D-positive NK cells after transduction with Mock (left panel), NKG2D-DAP10-CD3zeta (center panel) or NKG2D-41BB-CD3zeta (right panel) constructs. Mock transduced NK cell showed no NKG2D expression with the antibody used (which does not showing staining above an isotype-matched nonreactive antibody, despite the naturally high NKG2D expression on activated NK cells), while just under 60% of cells transduced with the NKG2D-DAP10-CD3zeta construct exhibited NKG2D expression above the isotype-matched non-reactive antibody control, and over 80% of NK cells transduced with the NKG2D-41BB-CD3zeta. Pooled data for the percentage of NKG2D positive NK cells from all donors is shown in FIG. 4B. Both engineered NKG2D constructs result in substantial gain in NKG2D expression compared to Mock, though there is not a significant difference between the percent expression of the two constructs. FIG. 4C depicts expression data based on Mean Fluorescence Intensity (MFI), which represents, within the population expressing the NKG2D construct, the degree to which that cell expresses the construct (e.g., multiple copies of the construct per cell would yield a greater MFI). By this measure, the expression of the NKG2D-41BB-CD3zeta is significantly greater than that of the NKG2D-DAP10-CD3zeta construct.

Collectively, these data demonstrate that, in accordance with several embodiments disclosed herein, engineered constructs can successfully be expressed on NK cells. In several embodiments, enhanced expression of the construct can be achieved by repeated transduction of the NK cells with a particular construct. In several embodiments, the components of the constructs can be delivered to a cell in a single vector, or alternatively using multiple vectors. Depending on the embodiment, the construct itself may lead to enhanced expression, for example a linear or head to tail construct may yield increased expression because of a lesser degree of in-cell assembly that a multiple subunit construct requires.

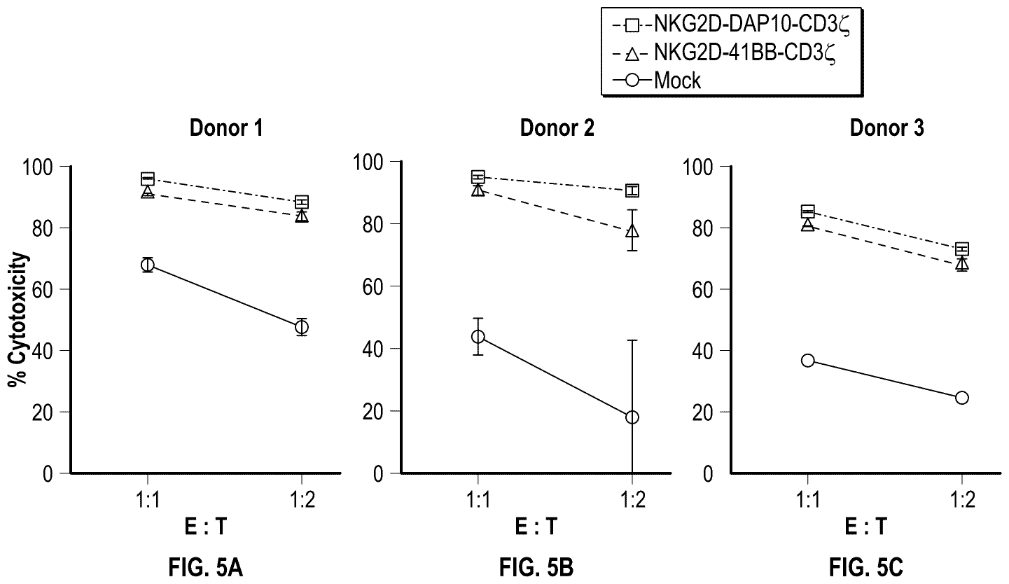

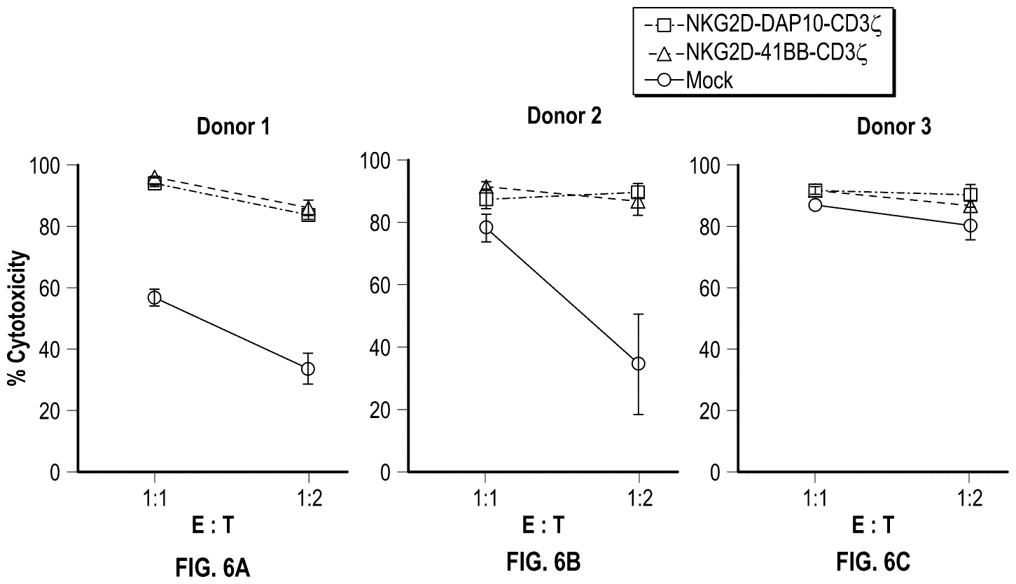

Further to successfully expressing NKG2D constructs on NK cells, effective signaling of the NK cells is required to act on target cells. To evaluate the potency of the two populations of transduced NK cells, cytotoxicity assays were performed using two cell lines that are sensitive to NK cell activity, REH (suspension cells) and U-2 OS (adherent cells). Data summarizing the percentage cytotoxicity of the different groups of NK cells against REH cells and across independent donors at two E:T ratios are shown in FIGS. 5A-5C (error bars represent standard deviation; all experiments are done in triplicates; n=3 (P <0.001)). As depicted in FIGS. 5A-5C, NK cells expressing either NKG2D chimeric receptor (NKG2D-DAP10-CD3zeta shown with an arrow labeled (a) and NKG2D-41BB-CD3zeta shown with an arrow labeled (b)) had a significantly higher cytotoxicity against REH for all three donors as compared to mock NK cells (shown with an arrow labeled (c)). The mean percentage cytotoxicity of NKG2D-DAP10-CD3zeta-expressing NK cells was 91.8%±5.8% (1:1 E:T ratio) and 83.9%±5.6% (1:2 E:T ratio). Those NK cells transduced with NKG2D-41BB-CD3zeta showed similar potencies—87.4%±6.1% at a 1:1 E:T ratio and 76.2%±4.8% at a 1:2 E:T ratio. Chimeric receptor-expressing NK cells also showed elevated cytotoxicity against U-2 OS when compared to mock-transduced NK cells (See FIGS. 6A-6C, FIG. 6A depicts NKG2D-DAP10-CD3zeta shown with an arrow labeled (a), FIG. 6B depicts NKG2D-41BB-CD3zeta shown with an arrow labeled (b) and FIG. 6C depicts mock NK cells shown with an arrow labeled (c)).

These data provide evidence that NK cells can not only be engineered to express chimeric receptor constructs, but those cells that express the chimeric receptors are able to be activated and successfully generate enhanced cytotoxic effects against target cells. Importantly, these data also show that there is only a slight decrease in the potency of the cells when in the presence of a greater number of target cells (doubled in this experiment). This suggests that the desired cytotoxic effects of the engineered NK cells can still be realized, even when the NK cells are present in smaller numbers vis-à-vis target cells, as would likely be the case in clinical use. Moreover, these data indicate that, according to some embodiments, a lesser density or degree of chimeric receptor expression on a given NK cell does not necessarily result in coordinately reduced cytotoxic effects, and can be associated with an unexpected efficacy of the NK cells in view of their lesser construct expression. Additionally, these data embody the unexpectedly enhanced cytotoxicity that is achieved according to several embodiments. While non-engineered NK cells are cytotoxic, and express a significant amount of NKG2D upon activation, it is unexpected that the engineered cells disclosed herein can push the cytotoxic effects significantly beyond what can be considered an already elevated ceiling (e.g., native NK cell cytotoxicity).

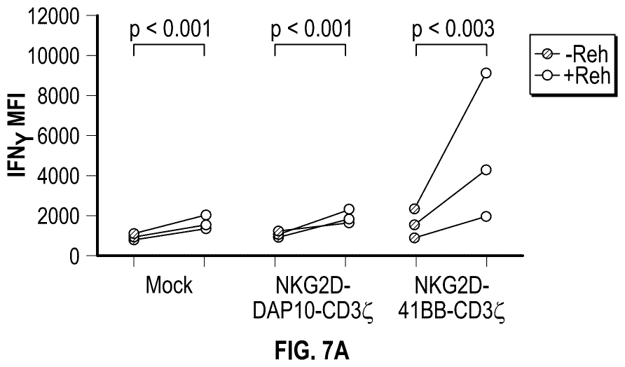

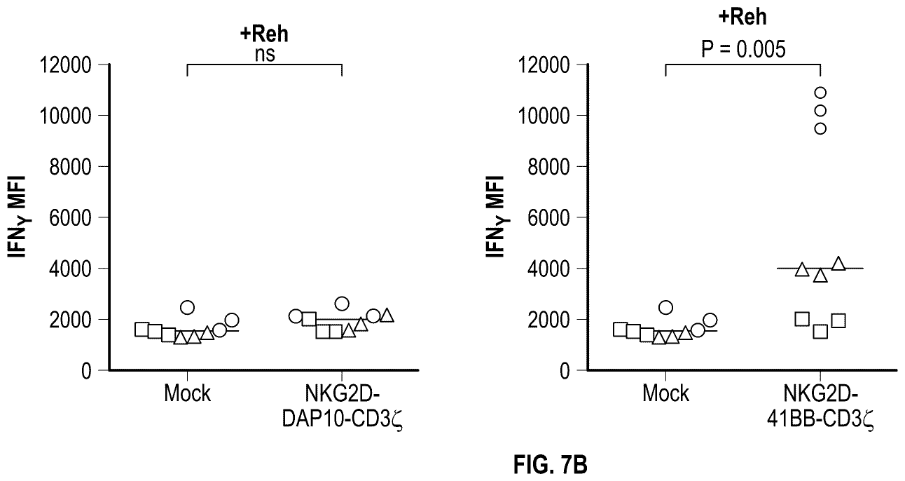

Further to the cytotoxicity data, the mechanism by which the NK cells are exerting these effects was examined, by evaluating the production if interferon-gamma (IFNγ) by the NK cells expressing the various NKG2D constructs. IFNγ is a key cytokine produced and released by NK cells (typically during an innate immune response) that recruits macrophages and has immunostimulatory effects. FIG. 7A shows the relative amount of IFNγ production (measured by MFI) in Mock (left panel), NKG2D-DAP10-CD3zeta-expressing NK cells (center panel), and NKG2D-41BB-CD3zeta-expressing NK cells (right panel) with or without stimulation by REH cells. NK cells were stained by APC-conjugated anti-IFNγ antibody for intracellular IFNγ. Data was analyzed by paired t test. These data show that each of the three groups of NK cells were observed to have a similar level of IFNγ production without stimulation, with an increase observed after stimulation by REH cells. As provided for in several embodiments, engineered NK cells expressing NKG2D constructs can lead to robust cytokine production. The presence of a target cell (here, REH cells) to which the engineered NK cells responds sets into motion the biochemical cascade which leads to IFNγ production and ultimately cytotoxic effects. As shown in FIG. 7A, the NKG2D-41BB-CD3zeta-expressing NK cells show a robust production of IFNγ in the presence of stimulatory REH cells. Interestingly, the NKG2D-DAP10-CD3zeta-expressing NK cells failed to show a similar degree of response. This is further demonstrated in FIG. 7B, where levels of IFNγ between different groups of NK cells after stimulation with REH cells (median values were represented; data was analyzed by unpaired t test) are evaluated. All IFNγ experiments were conducted in triplicates, with three independent donors, n=9. FIG. 7B shows that IFNγ production by NKG2D-DAP10-CD3zeta-expressing NK cells was not significantly different from mock-transduced NK cells. In contrast, the NKG2D-41BB-CD3zeta-expressing NK cells show a significant increase in IFNγ production as compared to mock-transduced NK cells. These data are interesting because they demonstrate that, as discussed herein, signaling by a chimeric receptor in response to ligand binding is an essential step in generating cytotoxic effects against a target cell of interest. However, there is not a singular pathway through which the various constructs signal, as NK cells transduced with two different chimeric receptors both exhibit relatively similar cytotoxicity, but without mirroring levels of IFNγ production. Thus, according to some embodiments, constructs are provided that achieve cytotoxic effects through an elevated production of IFNγ, or other immunostimulatory cytokine, as compared to normal NK cells. However, in several embodiments, increased production of IFNγ is not necessarily achieved or detected, rather another immunostimulatory pathway can be exploited by a given chimeric construct to achieve elevated cytotoxic effects.

### Example 2—CD16 and CD16-4-1BB Containing NKG2D Constructs

Additional constructs were generated to evaluate expression, cytotoxicity and cytokine production. As provided for herein, several embodiments relate to constructs comprising a truncated NKG2D (in some embodiments codon optimized), that employ a CD16 transmembrane and/or signaling domain. The constructs generated for evaluation in this experiment are schematically shown in FIGS. 2A-2B, which show the structure of A) NKG2D-CD16 and B) NKG2D-CD16-41BB chimeric receptors. Both chimeric receptors rely on the transmembrane region of CD16 to associate with either CD3zeta or FcRγ. The plasmids used to generate these constructs are shown in FIG. 3B. As discussed above, in several embodiments, the constructs employed rely on endogenous expression of CD3zeta or FcRγ, however, in several embodiments the plasmid encoding the chimeric receptor (or a separate plasmid) is configured to elevate expression of CD3zeta and/or FcRγ by the NK cell, thereby enhancing the potency of the cells.

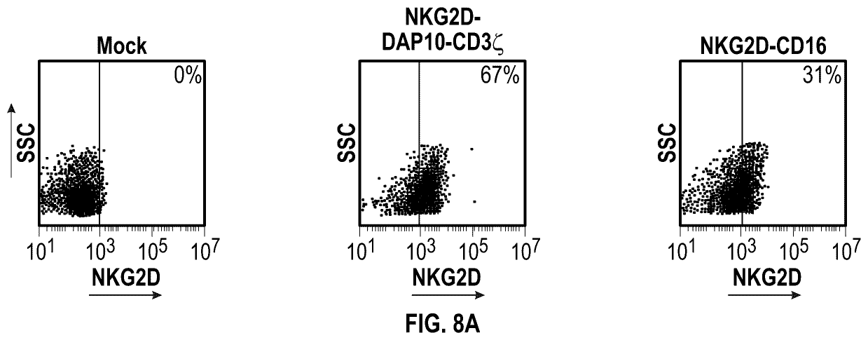

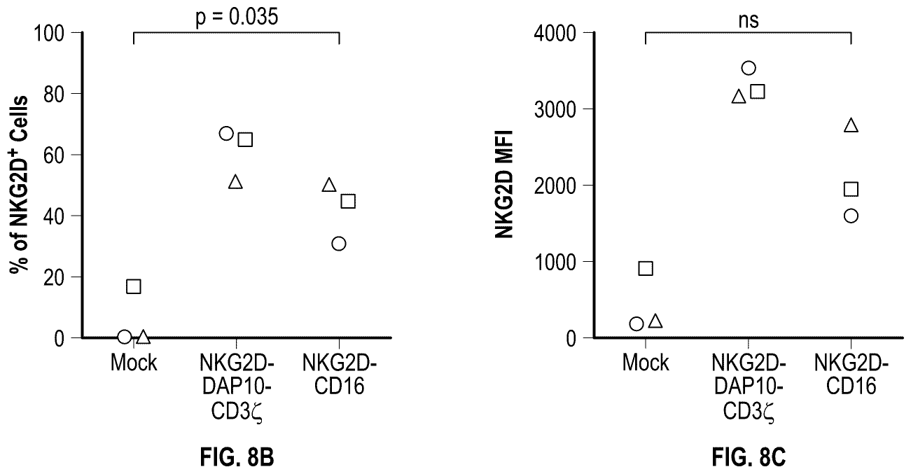

As above, expression levels of the constructs were evaluated. FIG. 8A depicts representative flow cytometry data for mock (left panel), NKG2D-DAP10-CD3zeta-expressing NK cells (center panel), and NKG2D-CD16-expressing NK cells (Experiments were conducted using cells from three independent donors represented by different symbols. Data was analyzed by paired t test). FIG. 8B shows summary data relating to the percentage of cells that that express NKG2D (and hence the constructs). As expected, mock-transfected NK cells show low levels of NKG2D expression with the antibody used. In contrast, both of the engineered constructs exhibited significantly enhanced expression, with NKG2D-CD16-transduced NK cells expressing 35.8%±6.9% greater expression as compared to mock-transduced NK cells. Additionally, as evaluated by MFI (FIG. 8C), NKG2D-CD16-transduced NK cells also exhibited increased expression of the construct. These data are important to demonstrate that the constructs can effectively be introduced into NK cells and are expressed.

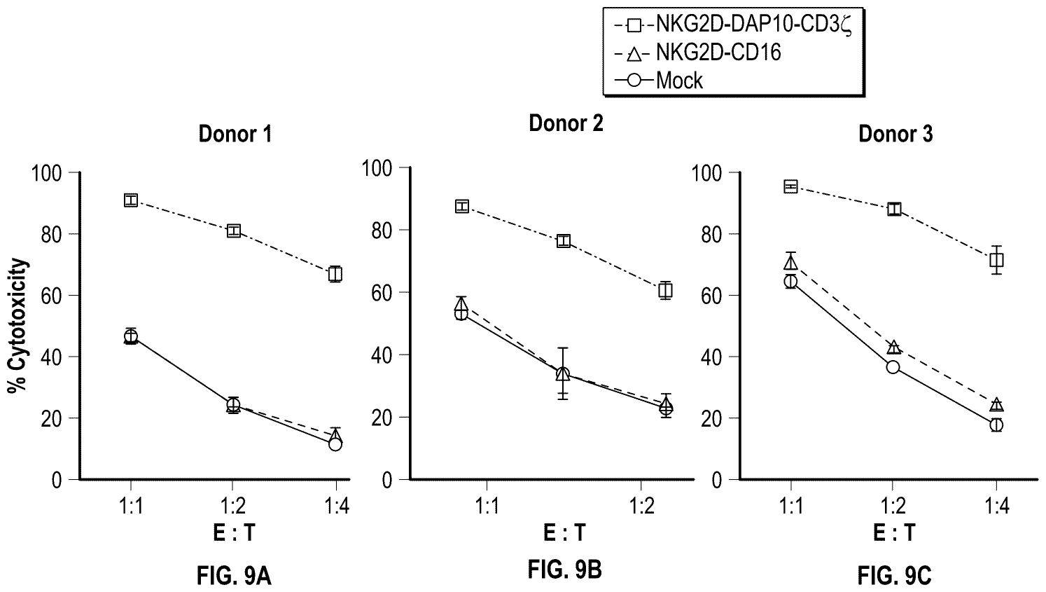

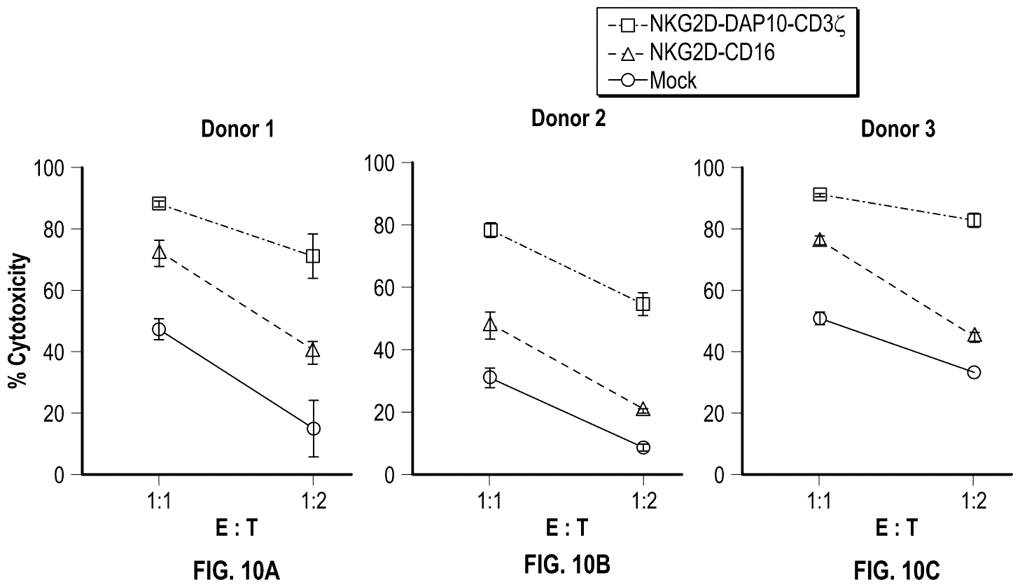

Having established expression of the constructs, their ability to exhibit cytotoxic effects was evaluated. As discussed above, NK cells from three donors were tested for cytotoxic effects against REH cells and U-2 OS cells, each at three E:T ratios (all experiments were done in triplicate, n=3). Interestingly, the enhanced expression of the NKG2D-CD16 construct as compared to mock NK cells did not result in increased cytotoxicity (see FIG. 9A-9C, error bars represent standard deviations). As with the prior example, NKG2D-DAP10-CD3zeta-expressing NK cells (shown with an arrow labeled (a)) did exhibit an increased cytotoxicity. With respect to cytotoxicity against U-2 OS cells, the NKG2D-CD16 (shown with an arrow labeled (b)) did exhibit an increased cytotoxicity as compared to mock NK cells (shown with an arrow labeled (c)) (see FIGS. 10A-10C). These data indicate that the degree of cytotoxic impact on a particular given target cell type may vary with the NK construct used. In some embodiments, a particular construct may not be as effective, however, in several embodiments, combinations of populations of NK cells can be used and exhibit synergistic effects. In other words, a population of NK cells, with a portion expressing NKG2D-CD16 and a portion expressing NKG2D-DAP10-CD3zeta (or other combination of any of the constructs disclosed herein), may exhibit unexpectedly enhanced cytotoxicity as compared to either sub-population alone.

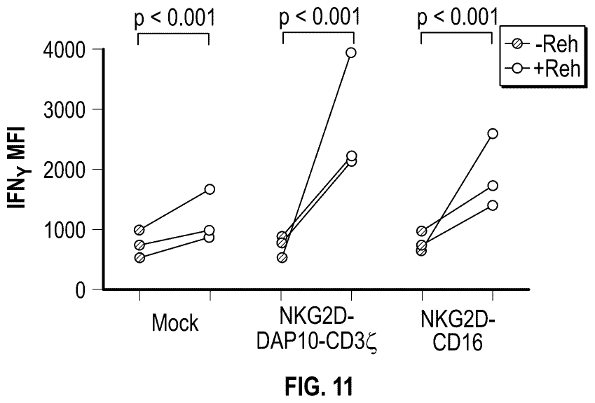

Interferon-γ production was measured next, in order to confirm the mechanism of action of the transfected NK cells. The NK cells expressing the various constructs were either stimulated by REH cells, or not, and the production of IFNγ was measured. These data are shown in FIG. 11 (data was analyzed by paired t test). All groups of NK cells had similar level of IFNγ without stimulation, and an increase after incubation with REH cells. The NKG2D-CD16-expressing NK cells exhibited an increase in IFNγ production of 634±211 MFI, which was greater than the increase exhibited by the mock-transduced NK cells (423±70 MFI). However, the increase was lower than that observed for NKG2D-DAP10-CD3zeta-expressing NK cells, which increased 2041±411 MFI. In line with data, according to several embodiments the production of IFNγ is correlated with the cytotoxic effects that NK cells expressing certain constructs exhibit.

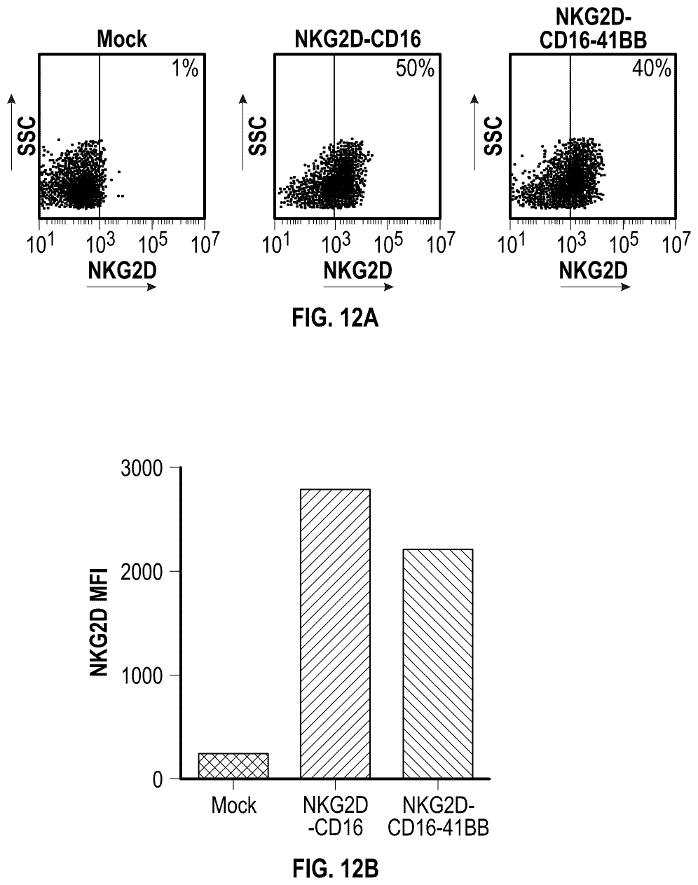

In accordance with several embodiments disclosed herein, multiple signaling regions may be used. Additional experiments were conducts to evaluate the expression of a NKG2D-CD16-41BB in expanded NK cells (experiments were conducted using cells from one donor). The expression data is shown in FIGS. 12A-12B. FIG. 12A shows raw flow cytometry data that demonstrate that the addition of the 4-1BB signaling region does not significantly impair the expression of the construct by NK cells, as compared to the NKG2D-CD16 construct. This is also reflected in the summary histogram of FIG. 12B that shows the relative amount of NKG2D receptors on the surface of each of the NK cell groups tested. The NKG2D-CD16-41BB shows slightly reduced MFI as compared to NKG2D-CD16, but both constructs show elevated expression versus mock.

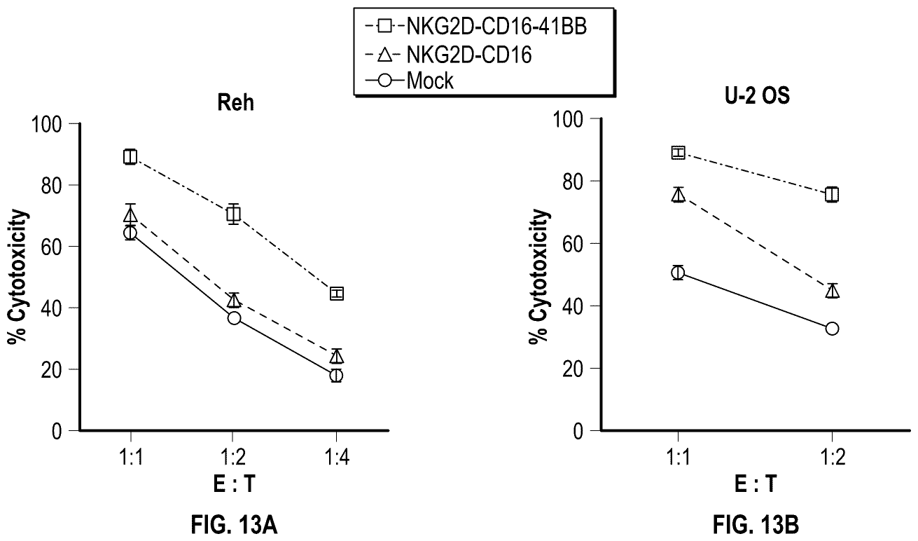

Cytotoxic effects were evaluated as described above, using both REH and U-2 OS cells as targets. FIGS. 13A-13B depict the resultant data (error bars represent standard deviations; all experiments were conducted in triplicates, n=3). FIG. 13A shows the cytotoxic effects of the constructs against REH cells. Similar to the experiment above, the NKG2D-CD16-expressing cells shown with an arrow labeled (b)) did not show significantly elevated cytotoxic effects as compared to mock NK cells shown with an arrow labeled (a). In contrast, NK cells expressing NKG2D-CD16-41BB (shown with an arrow labeled (c)) showed enhanced cytotoxicity against REH cells. With respect to efficacy against U-2 OS cells, both the NKG2D-CD16 and NKG2DCD16-41BB expressing cells showed enhanced cytotoxicity, with the NKG2D-CD16-41BB expressing cells exhibiting a more robust cytotoxic effect. This demonstrates that, in accordance with several embodiments, use of a combination of signaling domains can result in unexpected enhancements in the efficacy of a transduced NK cell. Thus, as described above, several embodiments employ two or more transmembrane/signaling domains that work synergistically together to yield enhanced cytotoxicity against target cells.

### Example 3—Additional NKG2D Constructs

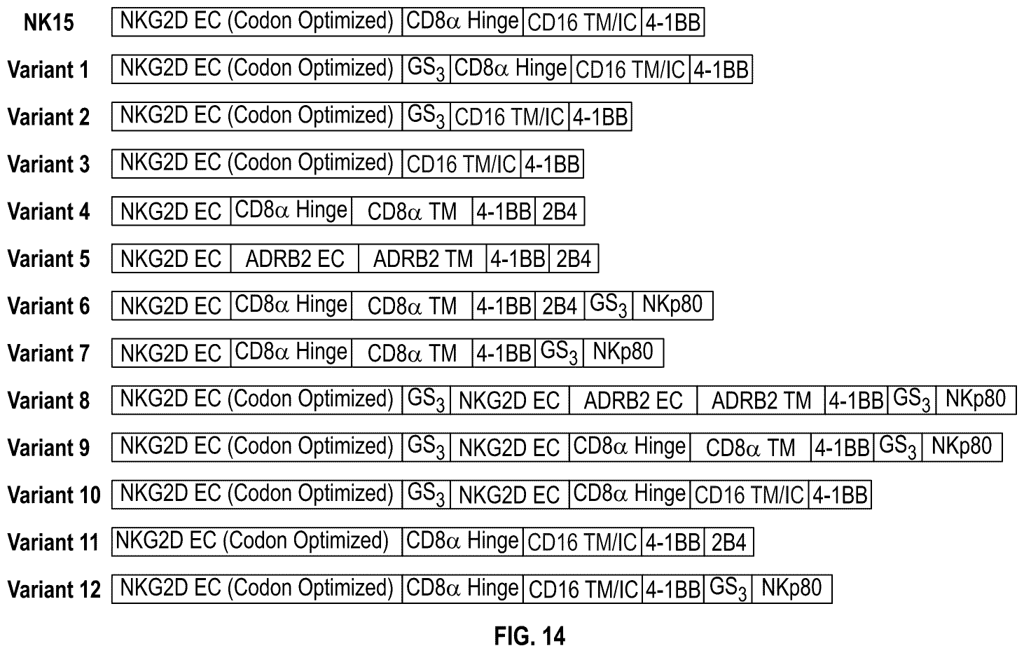

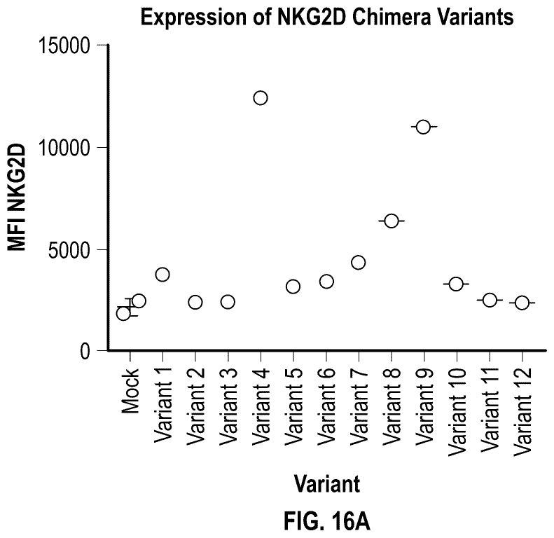

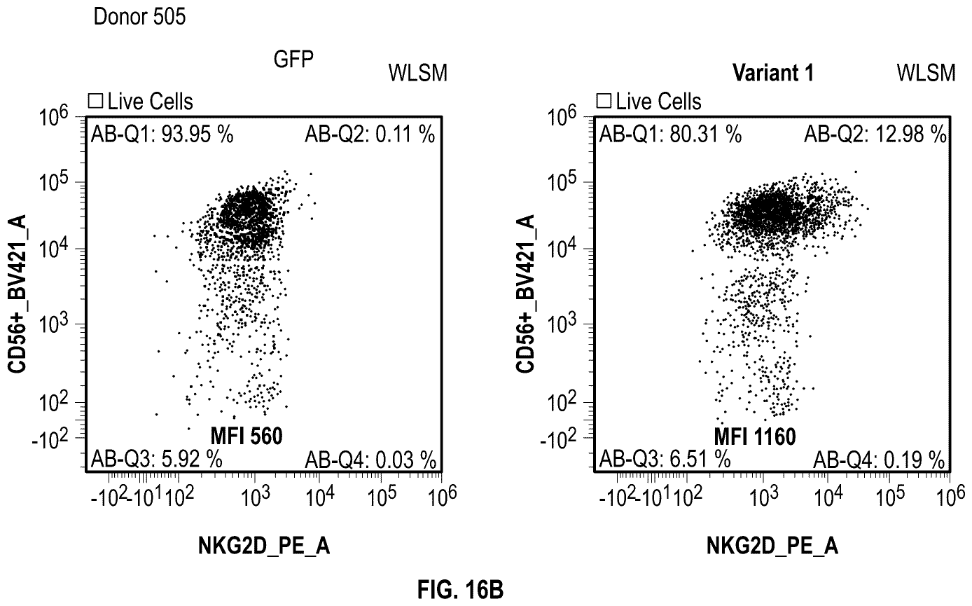

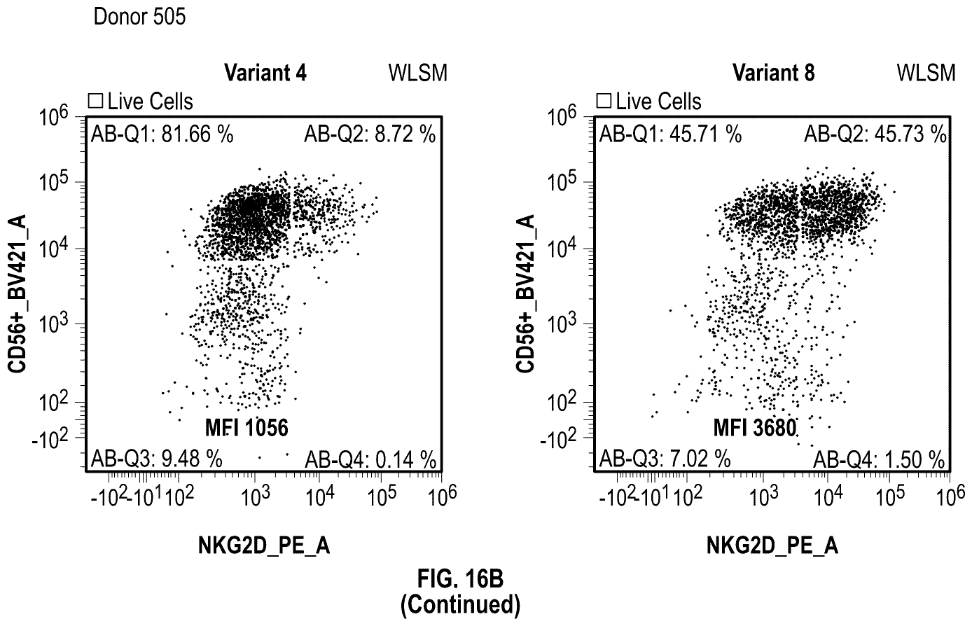

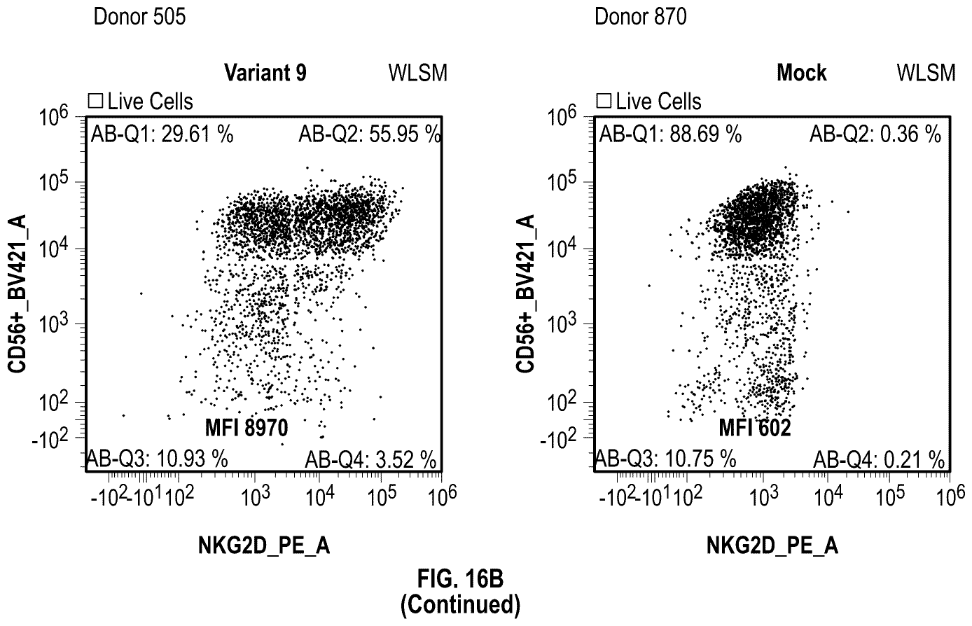

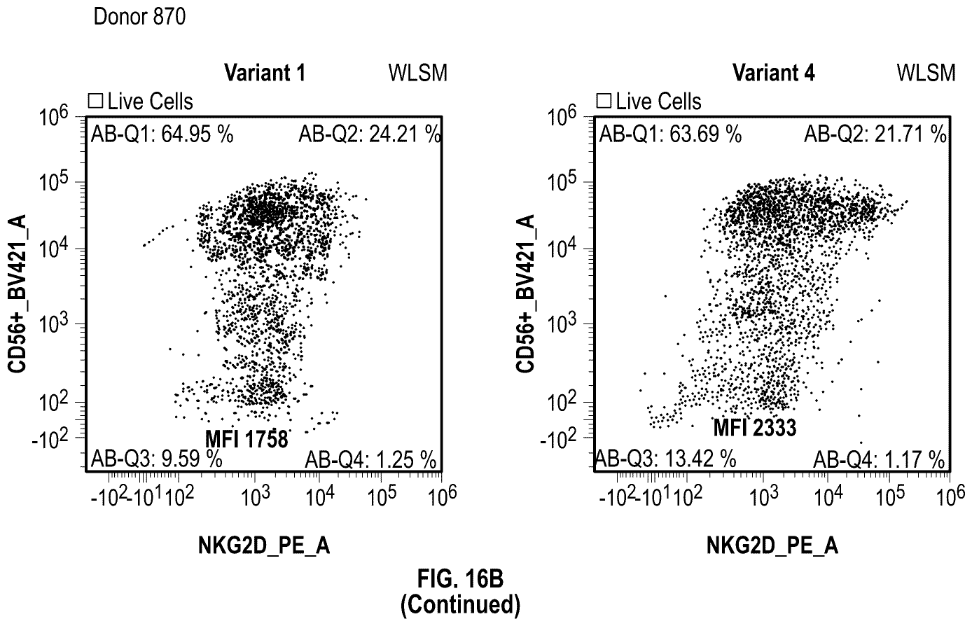

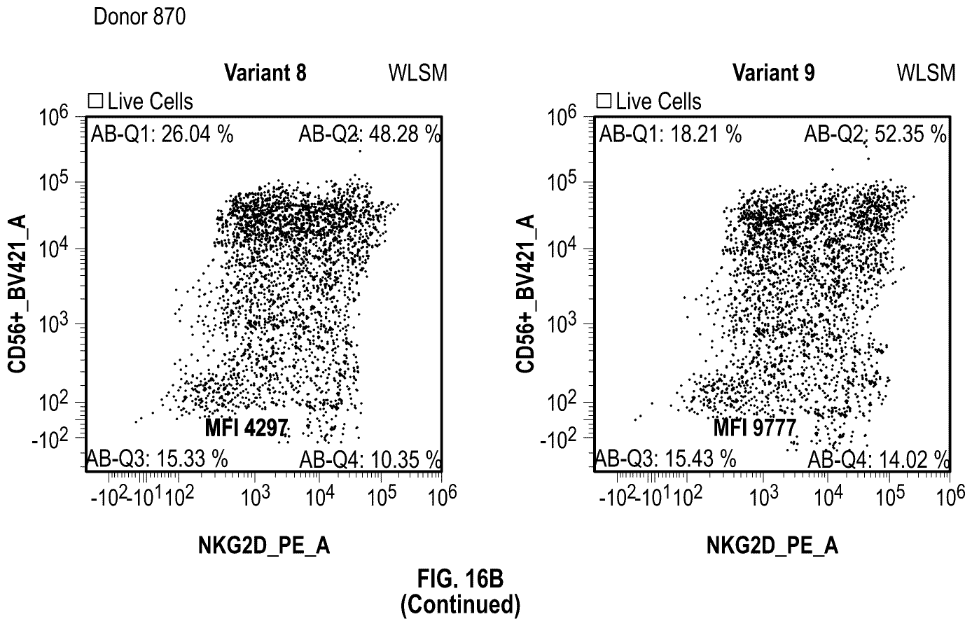

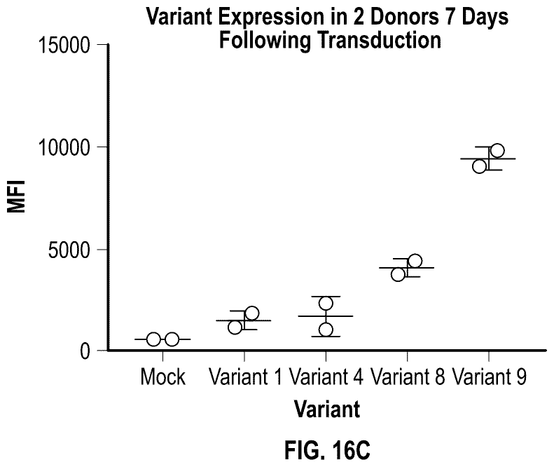

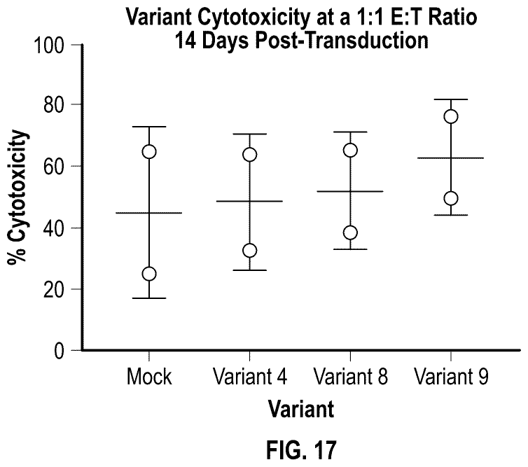

Additional constructs with varying extracellular domains, transmembrane domains, and intracellular effector domains were generated to evaluate their expression and cytotoxicity. The 12 constructs generated for evaluation in this experiment are schematically shown in FIG. 14. Some of these variant chimeric receptors rely on a CD16 transmembrane region to associate with either CD3zeta or FcRγ. As discussed above, in several embodiments, the constructs employed rely on endogenous expression of CD3zeta or FcRγ, however, in several embodiments the plasmid encoding the chimeric receptor (or a separate plasmid) is configured to elevate expression of CD3zeta and/or FcRγ by the NK cell, thereby enhancing the potency of the cells. As above, expression levels of the constructs were evaluated. Mock-transfected NK cells show low levels of NKG2D expression as evaluated by MFI (FIG. 16A). In contrast, NK cells transduced with the variant NKG2D constructs described above showed varying levels of NKG2D expression, with engineered variant constructs 4 and 9 exhibiting significantly enhanced expression in NK cells. FIG. 16B depicts representative flow cytometry data for variant NKG2D constructs 1, 4, 8, 9 after transduction into the NK cells of two donors. Relative to mock-transduced NK cells, Variant 8- and 9-transduced NK cells showed particularly strong expression of the chimeric receptor. Variant construct expression persisted in the NK cells of two donors 7 days following transduction, with Variants 8 and 9 showing particularly elevated levels as evaluated by MFI (FIG. 16C). These data are important to demonstrate that the constructs can effectively be introduced into NK cells and are expressed. Having established expression of the constructs, their ability to deliver cytotoxic effects in transduced NK cells was also evaluated. The cytotoxicity of the NKG2D variant constructs 4, 8, and 9 were evaluated 14 days post-transduction into NK cells at a 1:1 E:T ratio (FIG. 17).

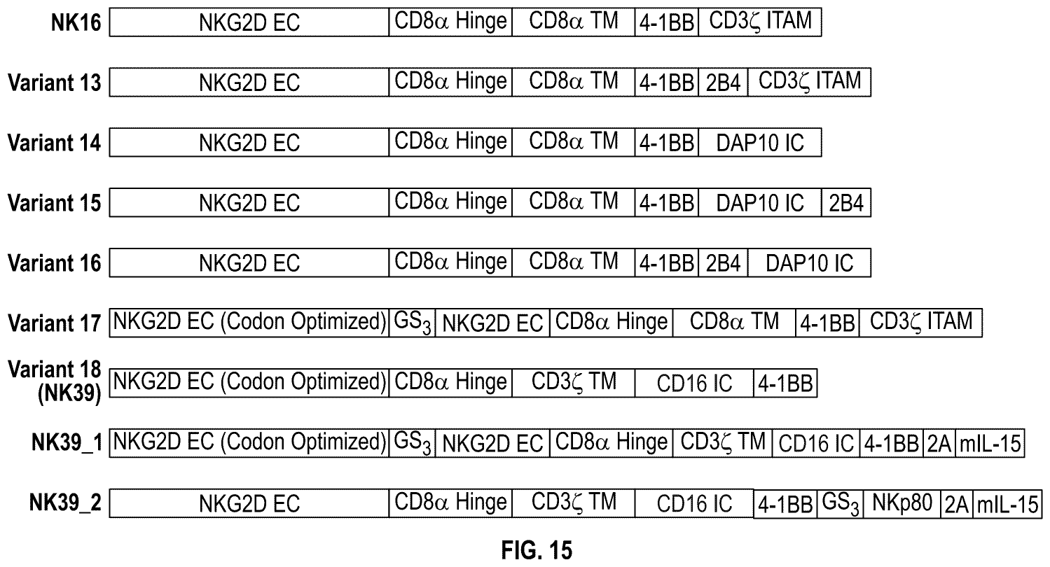

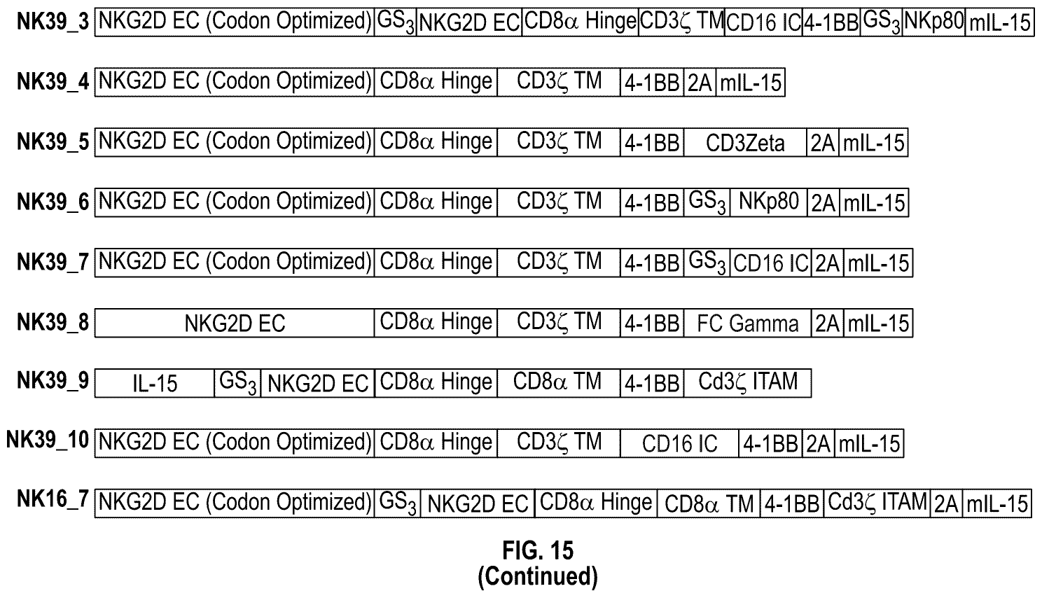

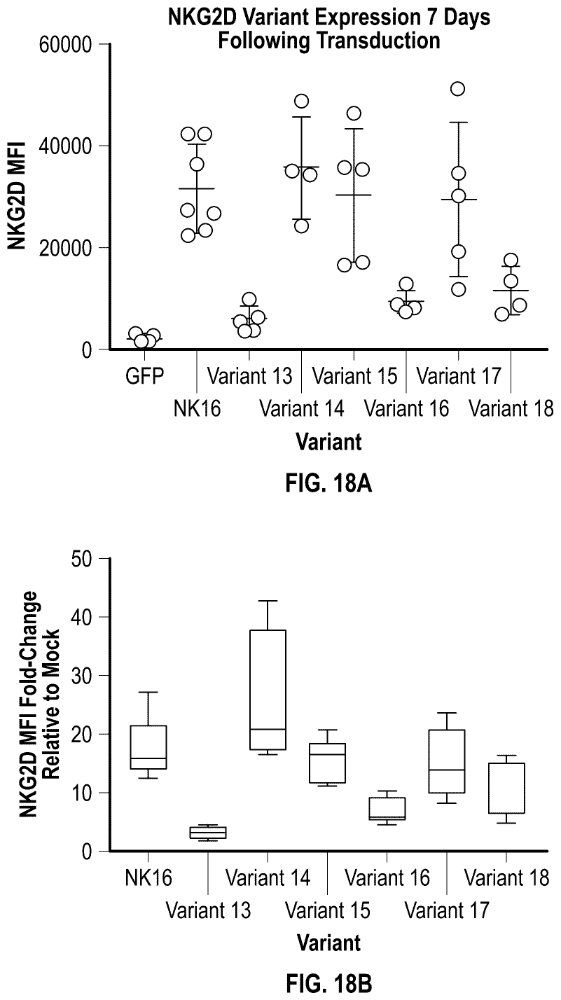

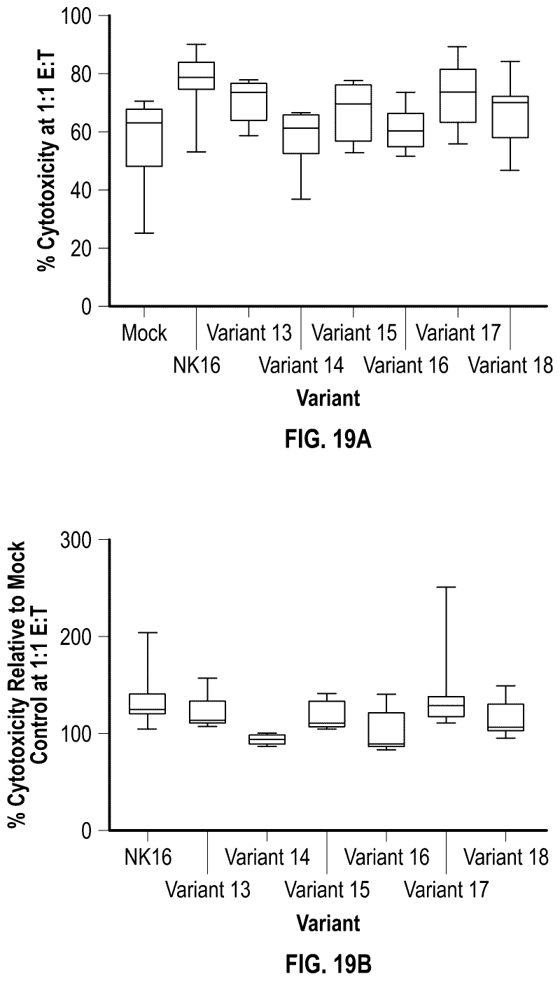

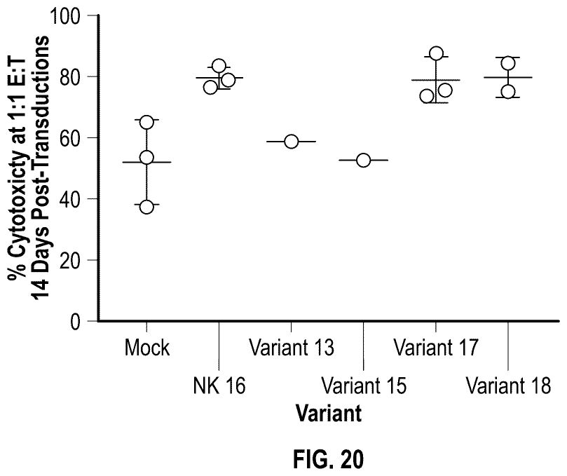

Further variant constructs were generated and are schematically shown in FIG. 15, which show the structure of chimeric receptors comprising various extracellular domains, transmembrane domains, and intracellular effector domains. Some of these variant chimeric receptors rely on an effector domain comprising CD3zeta and/or another signaling domain to transduce signaling upon ligand binding, while other variant chimeric receptors comprise a CD3zeta transmembrane domain that recruits full-length CD3zeta molecule to the synapse via dimerization. As above, expression levels of the constructs were evaluated. As evaluated by MFI (FIGS. 18A-B), NK cells transduced with engineered constructs exhibited increased expression of the chimeric receptor relative to mock transduced cells. Cytotoxic effects were evaluated as described above using an effector:target ratio of 1:1. As depicted in FIGS. 19A-B, NK cells transduced with engineered constructs (particularly variant 18) have enhanced cytotoxicity relative to the mock control.

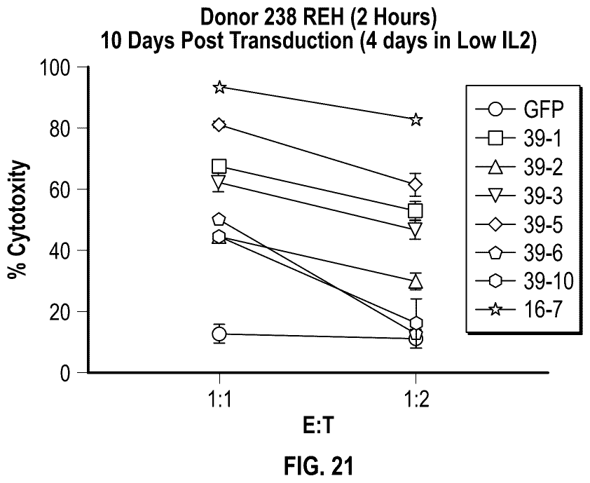

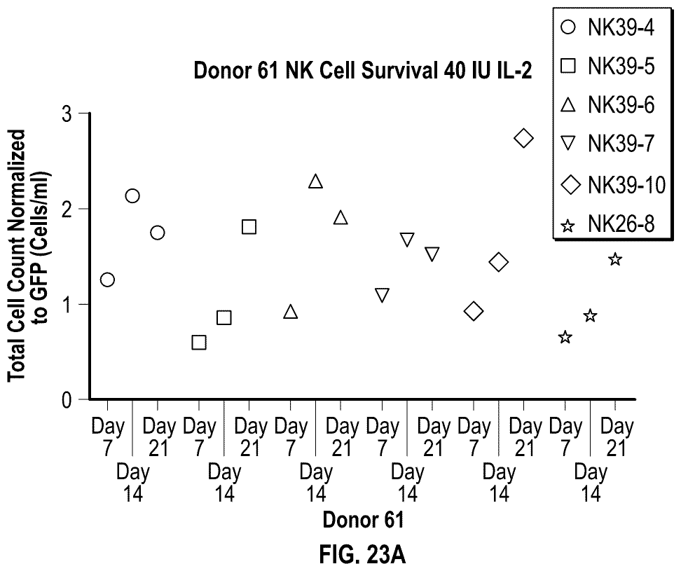

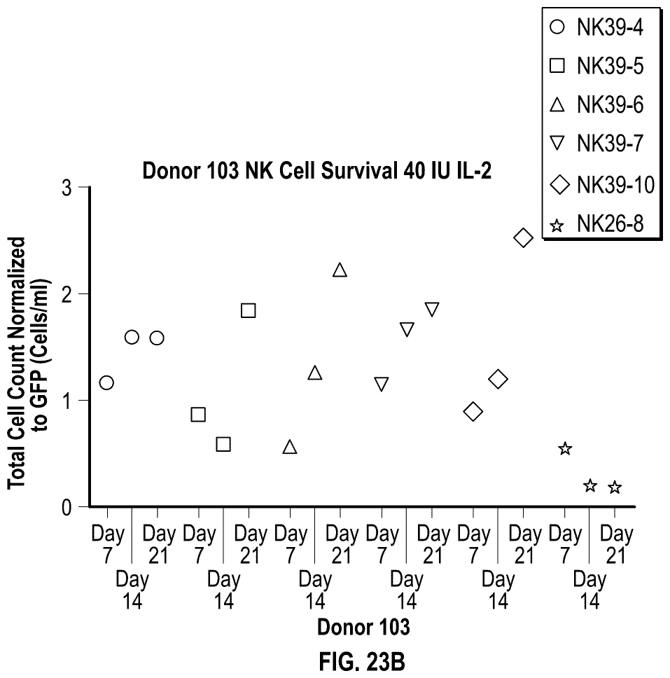

As variant 18 exhibited robust expression in NK cells that was accompanied by enhanced cytotoxic effects, a series of variant NKG2D constructs comprising a CD3zeta transmembrane domain were generated. These variants are termed “NK39” and are schematically shown in FIG. 15. Fourteen days following transfection into donor NK cells (with 4 days of culturing in low IL-2 conditions), the cytotoxicity of the transduced NK cells were evaluated. FIG. 21 shows the cytotoxic effects of the constructs against cultured REH cells at 1:1 and 1:2 E:T ratios. All the of the NK cells expressing engineered NK39 constructs showed significantly elevated cytotoxic effects as compared to control NK cells at a 1:1 E:T ratio. When evaluated at a 1:2 E:T ratio, chimeric constructs 16-7, 39-1, 39-2, 39-3, and 39-5 each enhanced the cytotoxic effects of their respective transduced NK cells relative to the mock control. As exogenous expression of activating receptors can lead to NK cell anergy and cell death, the engineered constructs were transduced into two donor NK cells and survival was evaluated after 21 days. As depicted in FIGS. 23A-B, NK39-5 and NK39-10 transduced cells show better survival than NK16 in two tested donors.

### Example 4—Evaluation of NK45 NKG2D Constructs

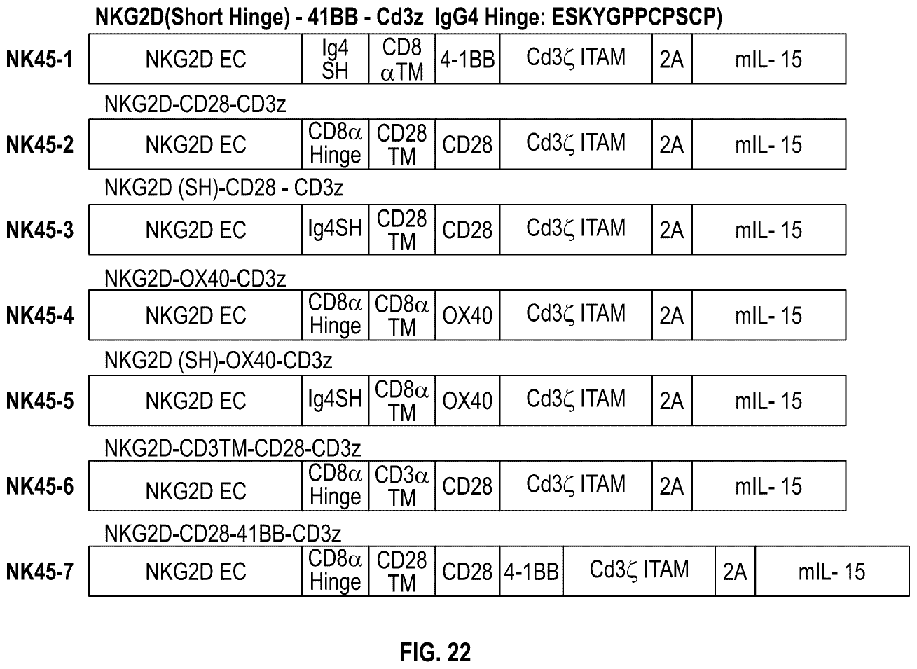

Additional constructs with varying extracellular domains, hinges, transmembrane domains, and intracellular effector domains according to embodiments disclosed herein are schematically shown in FIG. 22. The expression, cytotoxicity, persistence, and cytokine production mediated by these 7 constructs were evaluated in this Example relative to three of the NK39 constructs described in Example 3 (NK39-5, NK39-6, NK39-10) as well as a version of NK16 that bicistronically expresses membrane-bound interleukin 15 (NK26-8). In accordance with several embodiments disclosed herein, multiple signaling regions may be used. Some of these variant chimeric receptors rely on an effector domain comprising CD3zeta and/or another signaling domain (e.g., OX40, CD28, and/or 4-1BB costimulatory domains) to transduce signaling upon ligand binding, while other variant chimeric receptors comprise a CD3zeta transmembrane domain that recruits full-length CD3zeta molecule to the synapse via dimerization. As disclosed herein, these constructs are further configured to co-express membrane-bound IL15.

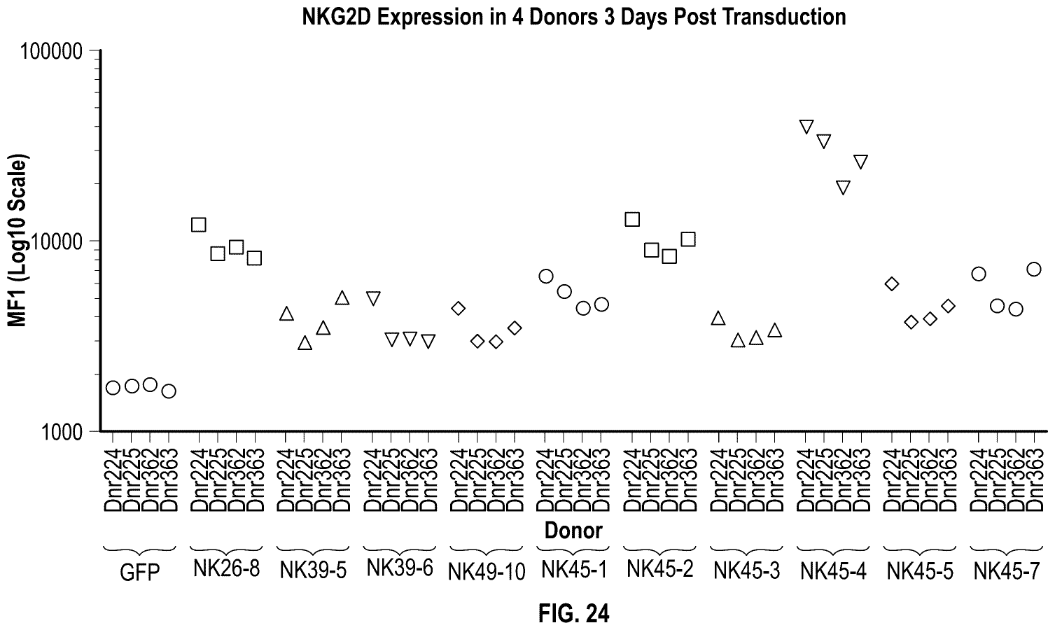

As above, the ability of NK cells to effectively express these constructs was first assessed. NK cells expanded from the PBMC of four donors were transduced with the variant constructs (or an empty MSCV control vector containing GFP only) and NKG2D expression was evaluated by MFI after 3 days. As depicted in FIG. 24, mock-transfected NK cells show relatively low levels of NKG2D expression. In contrast, the engineered variant constructs exhibited significantly enhanced expression, with NK45-4 (NKG2D-OX40-CD3zeta) showing surprisingly robust expression in all donors. OX40 is expressed in activated NK cells, but its role has not been well-established. A variant chimeric receptor with an effector domain containing a CD28 costimulatory domain (NK45-2; NKG2D-CD28-CD3zeta) also demonstrated robust expression 3 days post-transduction.

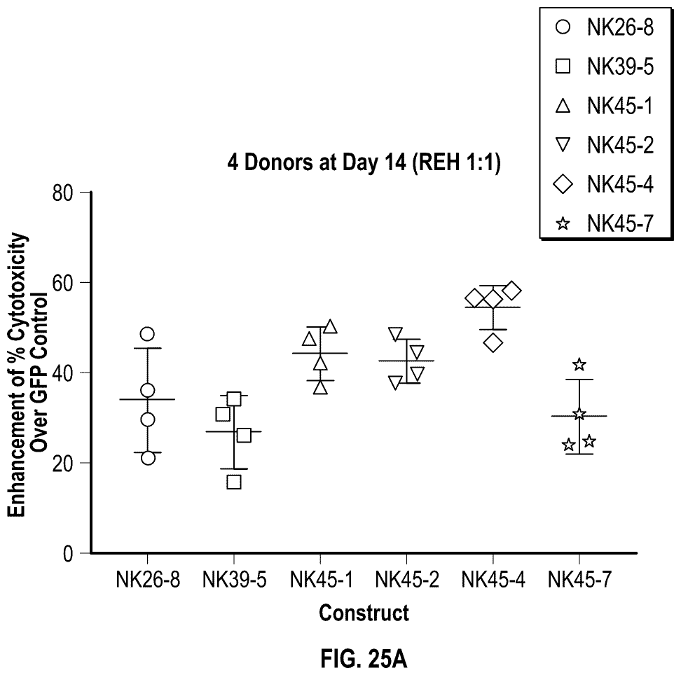

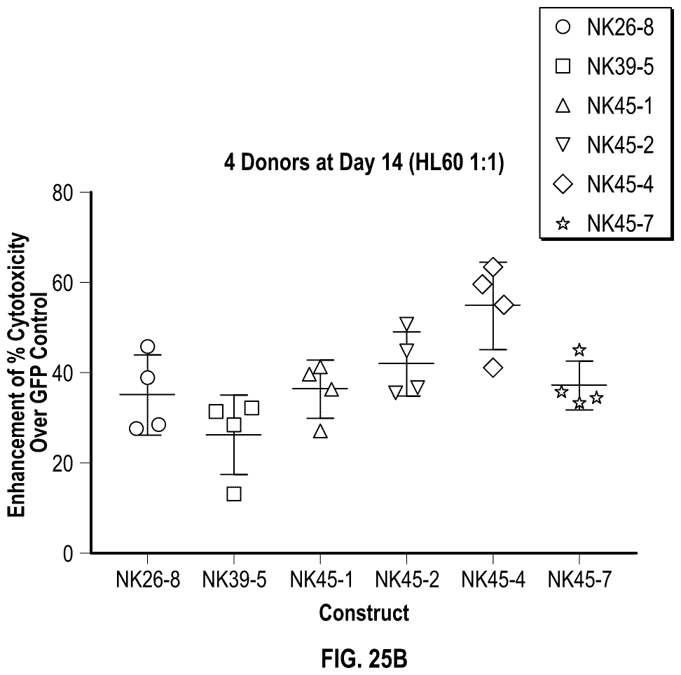

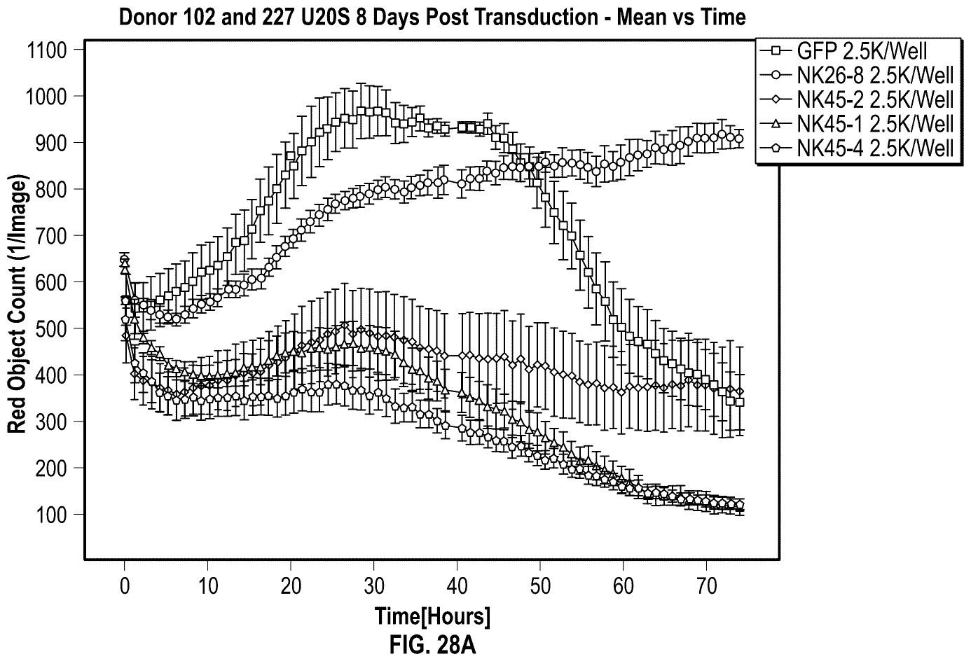

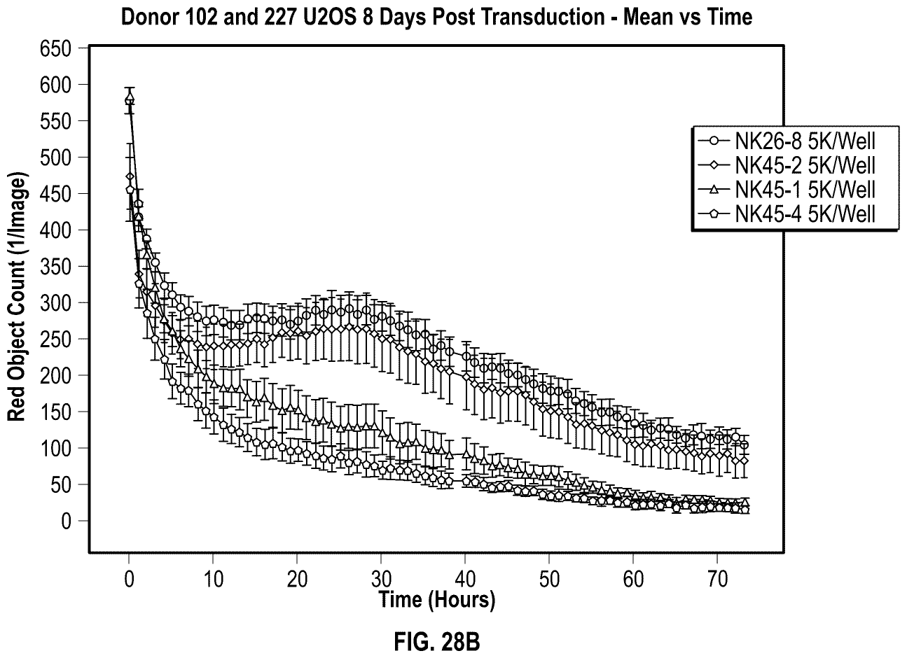

Having established expression of the variant constructs, their ability to exert cytotoxic effects was evaluated as above using REH and HL60 cells as targets. The potency of NK cells from four donors were examined against REH cells (FIG. 25A) and HL60 cells (FIG. 25B) at 1:1 E:T ratios 14 days post-transduction. As depicted in FIGS. 25A-B, the engineered constructs exerted an enhanced cytotoxicity against both REH and HL60 cells in all four donors as compared to mock NK cells. In addition to its pronounced expression profile, cells expressing NK45-4 (NKG2D-OX40-CD3zeta) also exhibited surprisingly elevated cytotoxicity relative to the mock control and the other constructs tested. NK cells expressing NK45-1 and NK45-2 also demonstrated pronounced cytotoxicity in these assays. These data demonstrate that, in accordance with several embodiments, use of a combination of signaling domains (particularly an OX40 costimulatory domain) can result in unexpected enhancements in the efficacy of a transduced NK cell. FIGS. 28A-B depict the cytotoxic activity against U2OS cells of the NK cells transduced with several of the variant constructs at various E:T ratios (1:2 and 1:4) and assessed over a more extended period of time. Surprisingly, NK cells transduced with the 45-4 construct appear to maintain cytotoxic activity through the time course. Advantageously, these experiments indicate that, according to several embodiments disclosed herein, the NKG2D variant constructs provide unexpectedly enhanced cytotoxicity over an extended period of time, which, depending on the embodiment, can range from 2-3 days, 3-5 days, 5-7, days, 7-8 days, 8-10 days, 10-14 days, 14-21 days, or 21-50 days (and any range in between those listed, including endpoints). In several embodiments, even longer durations of cytotoxic effects are achieved.

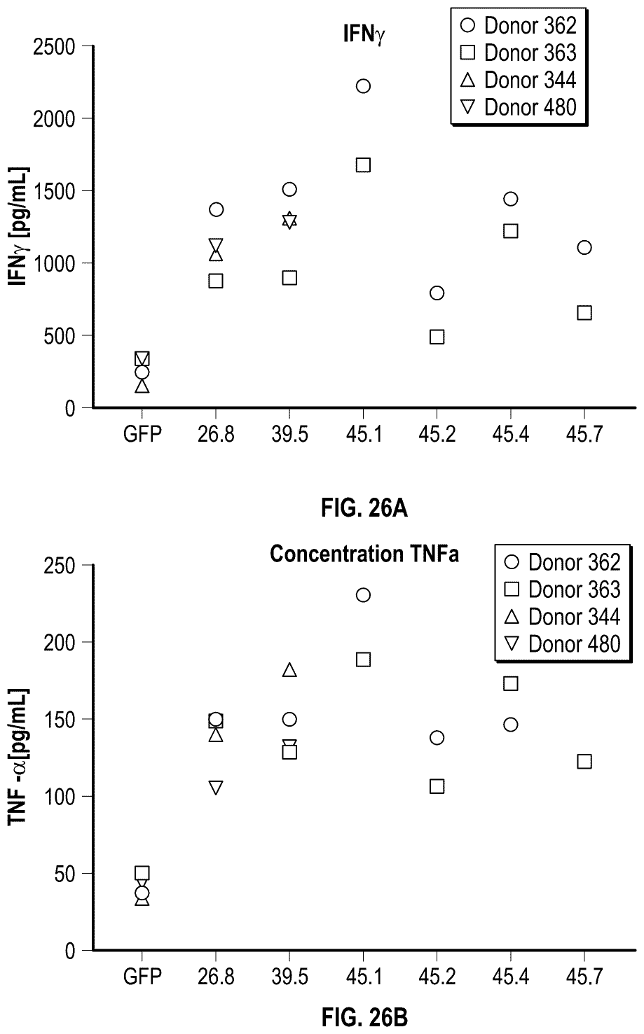

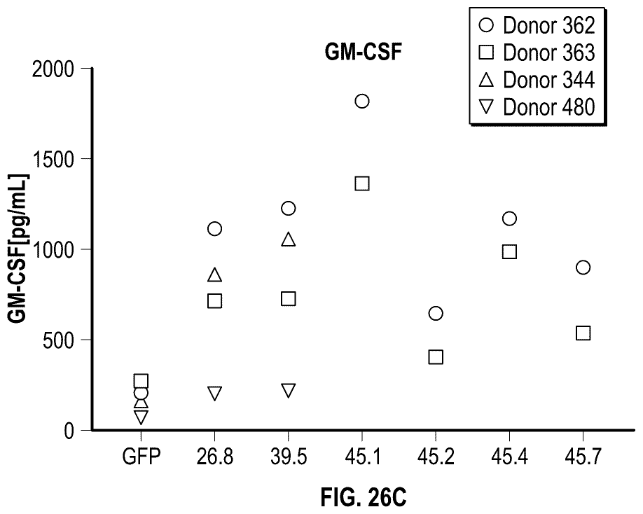

Further to the cytotoxicity data, the mechanism by which the NK cells are exerting these effects was examined by evaluating their production of IFNγ, TNFα, and GM-CSF following stimulation with REH cells. As depicted in FIGS. 26A-C, expression of each of the variant constructs yielded enhanced cytokine secretion relative to the production of IFNγ, TNFα, and GM-CSF exhibited by the GFP-expressing control NK cells. The chimeric receptor NK45-1 consistently mediated high cytokine production, which is surprising because this construct expresses at substantially lower levels than NK26-8 (from which it differs only with regards to the hinge region). Thus, these data demonstrate the unexpected importance of the hinge regions disclosed herein to mediating robust cytokine production in response to stimulation. Additionally, NKG2D-OX40-CD3zeta-expressing NK cells also showed an elevated production of IFNγ, TNFα, and GM-CSF.

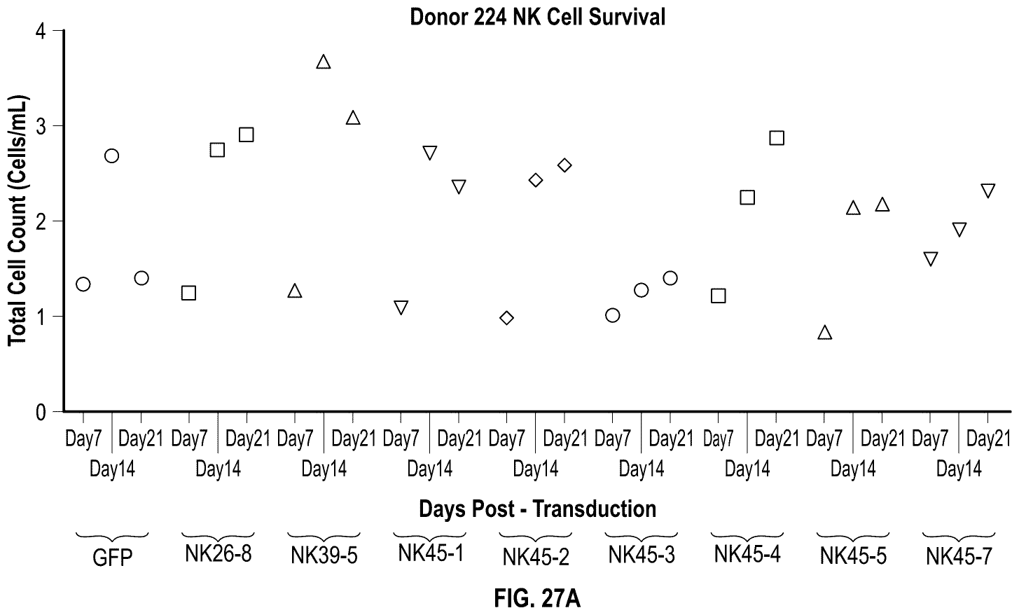

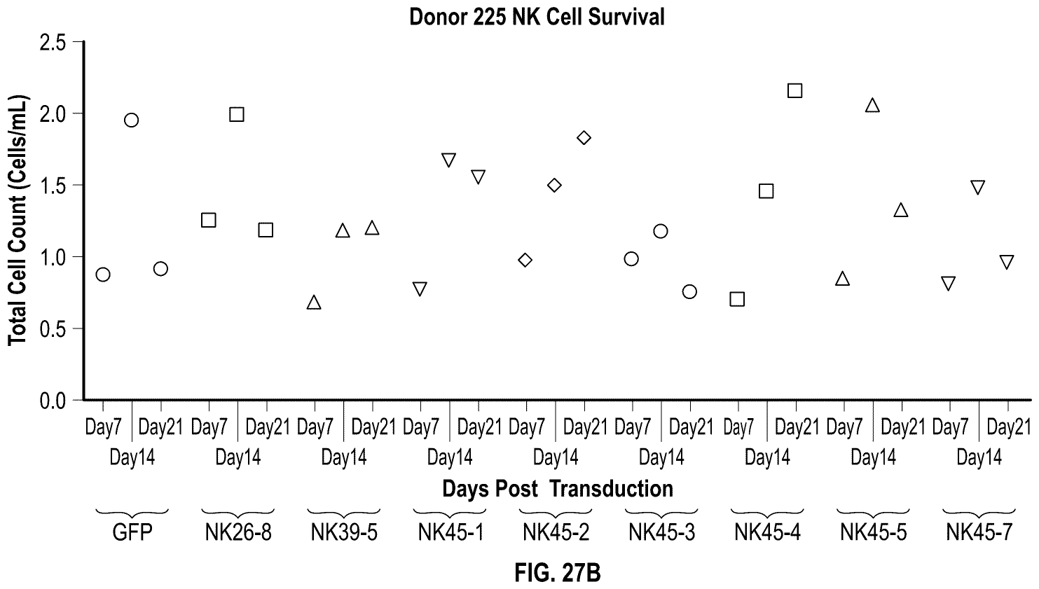

As exogenous expression of activating receptors can lead to NK cell anergy and cell death, the engineered constructs were transduced into two donor NK cells and the total cell count was evaluated 7, 14, and 21 days post-transduction. Surprisingly, the unexpectedly robust expression of NK45-4 does not come at the cost of reduced NK cell persistence in culture, as the total cell count remained at levels comparable to the GFP-expressing control cells (FIGS. 27A and 27B). Likewise, other NK cells expressing variant constructs at high levels continued to proliferate in the 2 donors for at least 3 weeks post-transduction. Collectively, these data demonstrate that, in accordance with several embodiments disclosed herein, engineered constructs can successfully be expressed at high levels in NK cells and mediate cytotoxic effects, and further, that this enhanced expression does not come at the detriment of reduced NK cell proliferation and/or survival.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a population of expanded NK cells” include “instructing the administration of a population of expanded NK cells.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

1. (canceled)

2. An immune cell expressing a chimeric receptor, the chimeric receptor comprising:
an extracellular receptor domain,
wherein said extracellular receptor domain comprises a peptide that binds native ligands of Natural Killer Group 2 member D (NKG2D),
wherein the peptide that binds native ligands of NKG2D is a fragment of NKG2D; and
an effector domain comprising a transmembrane region and an intracellular signaling domain,
wherein the transmembrane region comprises a CD8a hinge domain and a CD8a transmembrane domain, and
wherein the intracellular signaling domain comprises an OX-40 domain and CD3zeta, and
wherein the chimeric receptor comprises an amino acid sequence having at least 95% sequence homology to SEQ ID NO: 109.

3. The immune cell of claim 2, wherein the immune cell also expresses a membrane-bound interleukin 15 (mbIL15).

4. The immune cell of claim 3, wherein the chimeric receptor comprises the amino acid sequence of SEQ ID NO: 109.

5. The immune cell of claim 3, wherein in the mbIL15 comprises an amino acid sequence having at least 95% sequence homology to SEQ ID NO: 17.

6. The immune cell of claim 2, wherein the immune cell is a natural killer (NK cell).

7. An NK cell expressing a chimeric receptor, the chimeric receptor comprising:
(a) an extracellular receptor domain,
wherein said extracellular receptor domain comprises a peptide that binds native ligands of Natural Killer Group 2 member D (NKG2D),
wherein the peptide that binds native ligands of NKG2D is a fragment of NKG2D; and
(b) an effector domain comprising a transmembrane region and an intracellular signaling domain,
wherein the chimeric receptor has at least 95% sequence homology to the amino acid sequence of SEQ ID NO: 109.

8. The NK cell of claim 7, wherein the transmembrane region comprises a CD8a transmembrane domain, and wherein the intracellular signaling domain comprises an OX40 domain and a CD3zeta domain.

9. The NK cell of claim 7, wherein the NK cell further expresses membrane-bound interleukin 15 (mbIL15).

10. The NK cell of claim 7, wherein the chimeric receptor is encoded by a nucleic acid sequence encoding SEQ ID NO: 109.

11. The NK cell of claim 9, wherein mbIL15 comprises an amino acid having at least 95% homology to the amino acid sequence of SEQ ID NO: 17.

12. A method of treating cancer, the method comprising:
administering to a subject in need thereof a population of immune cells expressing a chimeric receptor, the chimeric receptor comprising:
an extracellular receptor domain,
wherein said extracellular receptor domain comprises a peptide that binds native ligands of Natural Killer Group 2 member D (NKG2D),
wherein the peptide that binds native ligands of NKG2D is a fragment of NKG2D; and
an effector domain comprising a transmembrane region and an intracellular signaling domain, and
wherein the chimeric receptor has at least 95% sequence homology to the amino acid sequence of SEQ ID NO: 109.

13. The method of claim 12, wherein the intracellular signaling domain comprises a CD3zeta signaling domain.

14. The method of claim 12, wherein the intracellular signaling domain comprises an OX40 domain.

15. The method of claim 12, wherein the intracellular signaling domain comprises an OX40 domain and a CD3zeta signaling domain, and wherein the immune cells also express a membrane-bound interleukin 15 (mbIL15).

16. The method of claim 12, wherein the immune cells are natural killer (NK) cells.

17. The method of claim 12, wherein the cancer is acute myeloid leukemia (AML).

18. The method of claim 16, wherein the cancer is acute myeloid leukemia (AML).

19. The method of claim 12, wherein the administration is intravenous or intraarterial.

20. The method of claim 12, wherein the population of immune cells administered is between 1×10⁶ to 1×10⁸ cells per kilogram of body weight of the subject.


SEQUENCE LISTING (109 sequences)

SEQ ID NO: 1 - DNA, 645 bp, Homo sapiens
  misc_feature 1..645: note = Full length NKG2D
  source 1..645: mol_type = genomic DNA
gggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 2 - DNA, 405 bp, Homo sapiens
  misc_feature 1..405: note = Truncated NKG2D
  source 1..405: mol_type = other DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 3 - DNA, 405 bp, Homo sapiens
  misc_feature 1..405: note = Codon Optimized Truncated NKG2D
  source 1..405: mol_type = other DNA
ctgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtg

SEQ ID NO: 4 - DNA, 63 bp, Homo sapiens
  misc_feature 1..63: note = CD8 Signaling Sequence
  source 1..63: mol_type = genomic DNA
atggctctgcccgtcaccgcactgctgctgcctctggctctgctgctgcacgccgcacgacca

SEQ ID NO: 5 - DNA, 135 bp, Homo sapiens
  misc_feature 1..135: note = CD8 alpha hinge
  source 1..135: mol_type = genomic DNA
accacaacccctgcaccacgcccccctacaccagcacctaccatcgcaagccagcctctgtccctgcggccagaggcatgtagaccagcagcaggaggagcagtgcacacaagaggcctggacttcgcctgcgat

SEQ ID NO: 6 - DNA, 1722 bp, Homo sapiens
  misc_feature 1..1722: note = CD8 beta
  source 1..1722: mol_type = genomic DNA
atctaggtcttgctgcacccgcacaacctacaaacagcgtcggggccttctctgcacctccagttcccagctcacctccctcagtgtcacagccggttacctttccttcctccctgggggagggcaagacttggggcttgctgactccaggcccagcccagcccggggcacccaggagcccctcaattgctactcaaacagacaagaagcggcccgagttagtggccagctccaccatgcactacacatcctgacctctctgagcctctactgtcactcggggtcacaaccctttcctgagcacctcccggggcagggggcgatgacacacatgcagctgcctgggggaggccggcggtgtcccctcctttctggaacgcggagggtcctggtgggctctggaaacgcagcccagacctttgcaatgctaggaggatgagggcggagacctcgcggtccccaacaccagactcccgcagccaccgcgcccggtcccgccctccccactgcccccccagctccccgacccaggcgccccgcccggccagctcctcacccaccccagccgcgactgtctccgccgagcccccggggccaggtgtcccgggcgcgccacgatgcggccgcggctgtggctcctcctggccgcgcagctgacaggtaaggcggcggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgctttcctcttccaggccggcggaggagagcccggcttcgtttcatgaaacagtaagtgtataacctgggtgtggccttgggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttgctgttgttttcagattttacaaatgagcagagaatacggttttggtgtcctgctacaaaaagacatcggtcagtaacgagcacgatgtggaaaaatgagagaagggacacattcaaccctggagagttcaatggctgctgaagctgcctgcttttcactgctgcaaggcctttctgtgtgtgacgtgcatgggagcaacttgttcgtgggtcatcgggaatactagggagaaggtttcattgcccccagggcacttcacagagtgtgctggaggactgagtaagaaatgctgcccatgccaccgcttccggctcctgtgctttccctgaactgggacctttagtggtggccatttagccaccatctttgcaggttgctttgccctggtagggcagtaacattgggtcctgggtctttcatggggtgatgctgggctggctccctgttggtcttcccaggctggggctgaccttcctcgcagagaggccaggtgcaggttgggaatgaggcttgctgagaggggctgtccagttcccagaaggcatatcagtctctgagggcttcctttggggccgggaacttgcgggtttgaggataggagttcacttcatcttctcagctcccatttctactcttaagtttctcagctcccatttctactctcccatggcttaatgcttctttcattttctgtttgttttatacaaatgtcttagttgtaaaaataaagtcccaggttaaagataacaaacgggtcctg

SEQ ID NO: 7 - DNA, 2415 bp, Homo sapiens
  misc_feature 1..2415: note = CD16 alpha
  source 1..2415: mol_type = genomic DNA
attcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaa

SEQ ID NO: 8 - DNA, 2473 bp, Homo sapiens
  misc_feature 1..2473: note = CD16 beta
  source 1..2473: mol_type = genomic DNA
aaagatgggtggagggactggggaaaggctgtttactccctcctgtctagtcggcttggtccctttaggggtccggatatctttggtgacttgtccactccagtgtggcatcatgtggcagctgctcctcccaactgctctgctacttctaggtaagtaggatctccctggttgagggagaagtttgagatgccttgggttcagcagagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagaggcatgaacagtggaagaccagagagcaggtagcaaggtttccaccagaaacatcctgattcttgggaaaattgggctcctggggcagaggagggcaggggagttttaaactcactctatgttctaatcactctgatctctgcccctactcaatatttgatttactcttttttcttgcagtttcagctggcatgcggactggtgagtcagcttcatggtcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactgagagctgagctcccgggcctggggtgtctctgtgtctttcaggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtgagacatgtgccaccctggaatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttttcatctctccacttctcctaataggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagtaaaacaacaatacaggctagggatggtaatcctttaaacatacaaaaattgctcgtattataaattacccagtttagaccggaaaaaagaaaataattattcctaaacaaatggataagtagaattaatgattgaggcaggaccctacagagtgtgggaactgctggggatctagagaattcagtgggaccaatgaaagcatggctgagaaatagcagggtagtccaggagagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcatgtcagttccaatgaggtggggatggagaagacaattgttgcttatgaaaaaaagctttagctgtctctgttttgtaagctttcagtgcaacatttcttggttccaataaagcattttaca

SEQ ID NO: 9 - protein, 370 aa, Homo sapiens
  REGION 1..370: note = MISC_FEATURE - 2B4
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 10 - DNA, 279 bp, Homo sapiens
  misc_feature 1..279: note = DAP10
  source 1..279: mol_type = genomic DNA
atgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagatggcaaagtctacatcaacatgccaggcaggggctga

SEQ ID NO: 11 - DNA, 575 bp, Homo sapiens
  misc_feature 1..575: note = DAP12
  source 1..575: mol_type = genomic DNA
agacttcctccttcacttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaacaaaaaaaaaaaaaaaaaa

SEQ ID NO: 12 - DNA, 126 bp, Homo sapiens
  misc_feature 1..126: note = 4-1BB
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 13 - DNA, 339 bp, Homo sapiens
  misc_feature 1..339: note = CD3-zeta
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 6 aa, Homo sapiens
  REGION 1..6: note = MISC_FEATURE - Canonical hemi-tam
DGYXXL

SEQ ID NO: 15 - skipped

SEQ ID NO: 16 - DNA, 614 bp, Homo sapiens
  misc_feature 1..614: note = Membrane-bound IL15
  source 1..614: mol_type = other DNA
atggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggtatcaccctttactgctaa

SEQ ID NO: 17 - protein, 204 aa, Homo sapiens
  REGION 1..204: note = MISC_FEATURE - Membrane-bound IL15
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 18 - DNA, 1140 bp, Homo sapiens
  misc_feature 1..1140: note = NKG2D/CD8a/4-1BB/CD3z
  misc_feature 1..1140: note = NKG2D/CD8a/4-1BB/CD3z (aka NK16)
  source 1..1140: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 19 - protein, 379 aa, Homo sapiens
  REGION 1..379: note = MISC_FEATURE - Amino acid sequence of NKG2D/CD8a/4-1BB/CD3z
  REGION 1..379: note = MISC_FEATURE - Amino acid sequence of NKG2D/CD8a/4-1BB/CD3z (aka NK16)
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 46 aa, Homo sapiens
  REGION 1..46: note = MISC_FEATURE - Amino acid for NCR1 TM/IC
MGLAFLVLVALVWFLVEDWLSRKRTRERASRASTWEGRRRLNTQTL

SEQ ID NO: 21 - protein, 276 aa, Homo sapiens
  REGION 1..276: note = MISC_FEATURE - Full length NCR2
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 22 - protein, 66 aa, Homo sapiens
  REGION 1..66: note = MISC_FEATURE - NCR3 TM/IC domains
AGTVLLLRAGFYAVSFLSVAVGSTVYYQGKCLTWKGPRRQLPAVVPAPLPPPCGSSAHLLPPVPGG

SEQ ID NO: 23 - DNA, 741 bp, Homo sapiens
  misc_feature 1..741: note = NKG2D/CD16
  source 1..741: mol_type = other DNA
atggccttaccagtgaccgccttgctcctgccgctggccttgctgctccacgccgcccgccccctgttcaaccaggaagtgcagatccccctgaccgagtcctattgtggcccttgccctaagaattggatttgctataaaaacaactgctaccagttctttgacgagtctaagaattggtatgagtcccaggcctcttgtatgagccagaacgcctctctgctgaaggtgtacagcaaggaggaccaggatctgctgaagctggtgaagtcctatcactggatgggcctggtgcacatccccacaaacggctcttggcagtgggaggacggctccatcctgtctcctaatctgctgaccatcatcgagatgcagaagggcgattgcgccctgtacgccagctccttcaagggctatatcgagaactgcagcacacccaatacctacatctgtatgcagcggacagtgaccacaaccccagcacccaggccccctacacctgcaccaaccatcgcaagccagccactgtccctgaggcctgaggcatgtaggccagcagcaggaggagcagtgcacacacggggcctggacttcgcctgcgatgtgagcttttgtctggtcatggtgctgctgttcgccgtggataccggcctgtatttttccgtgaagacaaatatccggtctagcaccagagactggaaggatcacaagttcaaatggaggaaggacccacaggacaag

SEQ ID NO: 24 - protein, 247 aa, Homo sapiens
  REGION 1..247: note = MISC_FEATURE - NKG2D/CD16
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDVSFCLVMVLLFAVDTGLYFSVKTNIRSSTRDWKDHKFKWRKDPQDK

SEQ ID NO: 25 - DNA, 870 bp, Homo sapiens
  misc_feature 1..870: note = CD8/NKG2DOpt/CD8a/CD16 TM/IC/4-1BB
  source 1..870: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 26 - protein, 289 aa, Homo sapiens
  REGION 1..289: note = MISC_FEATURE - CD8/NKG2DOpt/CD8a/CD16 TM/IC/4-1BB
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDVSFCLVMVLLFAVDTGLYFSVKTNIRSSTRDWKDHKFKWRKDPQDKKRGRKKLLYIFKQPFMRPVQTTQEEDGCSCRFPEEEEGGCEL

SEQ ID NO: 27 - DNA, 741 bp, Homo sapiens
  misc_feature 1..741: note = NKG2D/NCR1
  source 1..741: mol_type = other DNA
atggccttaccagtgaccgccttgctcctgccgctggccttgctgctccacgccgcccgccctctgttcaaccaggaagtgcagatccctctgaccgaaagctattgcggaccttgccctaagaattggatttgctataaaaacaactgctaccagttctttgacgagtctaagaattggtatgagtctcaggccagctgtatgtcccagaacgcctctctgctgaaggtgtacagcaaggaggaccaggatctgctgaagctggtgaagtcctatcactggatgggcctggtgcacatccccacaaacggctcttggcagtgggaggacggctctatcctgagccctaatctgctgaccatcatcgagatgcagaagggcgattgcgccctgtacgccagctccttcaagggctatatcgagaactgcagcacacccaatacctacatctgtatgcagaggacagtgaccacaaccccagcaccccgcccccctacacctgcaccaaccatcgcaagccagccactgtccctgcggcctgaggcctgcagaccagcagcaggaggagcagtgcacacccggggcctggacttcgcctgtgatatgggcctggcctttctggtgctggtggccctggtgtggtttctggtggaggattggctgtcccggaagagaacaagggagagggcctcccgggcctctacctgggaaggaagaaggagactgaacacccagacactg

SEQ ID NO: 28 - protein, 247 aa, Homo sapiens
  REGION 1..247: note = MISC_FEATURE - NKG2D/NCR1
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDMGLAFLVLVALVWFLVEDWLSRKRTRERASRASTWEGRRRLNTQTL

SEQ ID NO: 29 - DNA, 801 bp, Homo sapiens
  misc_feature 1..801: note = NKG2D/NCR3
  source 1..801: mol_type = other DNA
atggccttaccagtgaccgccttgctcctgccgctggccttgctgctccacgccgccagacccctgttcaaccaggaggtgcagattcccctgacagaaagctattgtggcccttgccctaaaaattggatttgctataaaaacaactgctaccagttctttgacgagtctaagaattggtatgagtctcaggccagctgtatgtcccagaacgcctctctgctgaaggtgtacagcaaggaggaccaggatctgctgaagctggtgaagtcctatcactggatgggcctggtgcacatccctacaaacggctcttggcagtgggaggacggctctatcctgagcccaaatctgctgaccatcatcgagatgcagaagggcgattgcgccctgtacgccagctccttcaagggctatatcgagaactgcagcacacccaatacctacatctgtatgcagcggacagtgaccacaaccccagcacccagaccccctacacctgcaccaaccatcgccagccagccactgtccctgaggcccgaggcatgcaggcctgcagcaggaggcgccgtgcacacaaggggcctggactttgcctgtgatgcaggaaccgtgctgctgctgagagcaggcttctatgccgtgtcctttctgtctgtggccgtgggctccacagtgtactatcagggcaagtgcctgacctggaagggcccacggagacagctgcccgccgtggtgcccgcccctctgccacccccttgtggcagtagcgcccacctgctgccacccgtgcccggagga

SEQ ID NO: 30 - protein, 267 aa, Homo sapiens
  REGION 1..267: note = MISC_FEATURE - NKG2D/NCR3
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDAGTVLLLRAGFYAVSFLSVAVGSTVYYQGKCLTWKGPRRQLPAVVPAPLPPPCGSSAHLLPPVPGG

SEQ ID NO: 31 - protein, 6 aa, Homo sapiens
  SITE 6: note = MISC_FEATURE - N is an integer indicating the number of GGGGS repeated
GGGGSN

SEQ ID NO: 32 - protein, 60 aa, Homo sapiens
  REGION 1..60: note = MISC_FEATURE - GS3/CD8a
GGGGSGGGGSGGGGSTTTPAPRPPTPAPTIASQPLSLRPEACRPAAGGAVHTRGLDFACD

SEQ ID NO: 33 - protein, 45 aa, Homo sapiens
  REGION 1..45: note = MISC_FEATURE - GS9
GGGGSGGGGSGGGGSGGGGSGGGGSGGGGSGGGGSGGGGSGGGGS

SEQ ID NO: 34 - protein, 15 aa, Homo sapiens
  REGION 1..15: note = MISC_FEATURE - GS3
GGGGSGGGGSGGGGS

SEQ ID NO: 35 - protein, 120 aa, Homo sapiens
  REGION 1..120: note = MISC_FEATURE - 2B4 ICR
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 36 - DNA, 360 bp, Homo sapiens
  misc_feature 1..360: note = 2B4 ICR
  source 1..360: mol_type = genomic DNA
tggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagc

SEQ ID NO: 37 - protein, 38 aa, Homo sapiens
  REGION 1..38: note = MISC_FEATURE - NKp80 ICR
MQDEDGYMTLNVQSKKRSSAQTSQLTFKDYSVTLHWYK

SEQ ID NO: 38 - DNA, 114 bp, Homo sapiens
  misc_feature 1..114: note = NKp80 ICR
  source 1..114: mol_type = genomic DNA
atgcaggatgaggacggctatatgaccctgaacgtccagtccaagaagaggtccagcgctcagaccagccagctgaccttcaaggactactccgtgaccctgcactggtacaag

SEQ ID NO: 39 - protein, 30 aa, Homo sapiens
  REGION 1..30: note = MISC_FEATURE - B2Ad N-term ECD
MGQPGNGSAFLLAPNRSHAPDHDVTQQRDE

SEQ ID NO: 40 - DNA, 90 bp, Homo sapiens
  misc_feature 1..90: note = B2 AdR N-term ECD
  source 1..90: mol_type = genomic DNA
atggggcaacccgggaacggcagcgccttcttgctggcacccaatagaagccatgcgccggaccacgacgtcacgcagcaaagggacgag

SEQ ID NO: 41 - protein, 33 aa, Homo sapiens
  REGION 1..33: note = MISC_FEATURE - B2 AdR TM helix
VWVVGMGIVMSLIVLAIVFGNVLVITAIAKFER

SEQ ID NO: 42 - DNA, 99 bp, Homo sapiens
  misc_feature 1..99: note = B2AdR TM helix
  source 1..99: mol_type = genomic DNA
gtgtgggtggtgggcatgggcatcgtcatgtctctcatcgtcctggccatcgtgtttggcaatgtgctggtcatcacagccattgccaagttcgagcgt

SEQ ID NO: 43 - DNA, 924 bp, Homo sapiens
  misc_feature 1..924: note = NK15_1
  source 1..924: mol_type = other DNA
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgggtggcggtggctcgggcggtggtgggtcgggtggcggcggatctaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 44 - DNA, 789 bp, Homo sapiens
  misc_feature 1..789: note = NK15_2
  source 1..789: mol_type = other DNA
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgggtggcggtggctcgggcggtggtgggtcgggtggcggcggatctgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 45 - DNA, 744 bp, Homo sapiens
  misc_feature 1..744: note = NK15_3
  source 1..744: mol_type = other DNA
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtggtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 46 - DNA, 1164 bp, Homo sapiens
  misc_feature 1..1164: note = NK15_4
  source 1..1164: mol_type = other DNA
gccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacaacccccgctcccagacctcctacccctgcccctacaatcgccagccagcccctgagcctgagacccgaagcctgtagacctgctgccggaggcgctgtgcacacaagaggcctggacttcgcctgcgatatctatatctgggcccctctggctggaacctgtggcgtgctgctgctgagcctggtgattaccaagaggggcaggaagaagctgctgtacatcttcaagcagcctttcatgaggcccgtgcaaaccacccaggaggaggacggctgcagctgcagattccctgaggaggaggagggcggatgcgagctgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagctga

SEQ ID NO: 47 - DNA, 1155 bp, Homo sapiens
  misc_feature 1..1155: note = NK15_5
  source 1..1155: mol_type = other DNA
gccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgatgggacagcctggaaacggcagcgccttcctgctggcccctaacagaagccacgcccccgatcacgatgtgacccagcagagggacgaggtgtgggtggtgggcatgggcatcgtgatgagcctgatcgtgctggctatcgtgttcggcaacgtgctggtgatcaccgccatcgccaagttcgagaggaagaggggcaggaaaaagctgctctacatcttcaagcagcccttcatgaggcccgtgcagaccacccaggaagaggatggctgctcctgtaggtttcccgaggaggaggagggcggctgtgagctgtggaggagaaaaaggaaggagaagcagagcgagaccagccccaaggagttcctgaccatctacgaggacgtgaaggacctgaagaccaggaggaaccacgagcaggaacagaccttccccggcggaggcagcaccatctacagcatgatccagagccagtccagcgcccccacaagccaggaacccgcctacacactgtatagcctgatccagccctccaggaagagcggcagcaggaagaggaaccacagccccagcttcaacagcaccatttacgaggtgatcggaaagagccagcccaaggctcagaaccccgccaggctgagcaggaaggagctcgaaaacttcgacgtgtacagctga

SEQ ID NO: 48 - DNA, 1349 bp, Homo sapiens
  misc_feature 1..1349: note = NK15_6
  source 1..1349: mol_type = other DNA
ggatccgaattcgccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacaacccctgctcccagacctcccacacccgcccctacaatcgcctcccagcctctgagcctgagacccgaagcctgtagacctgccgccggcggagctgtgcatacaagaggcctggacttcgcctgcgacatctacatctgggcccctctggctggcacatgcggagtcctgctgctgagcctggtgatcaccaagaggggcaggaagaagctgctgtacatcttcaagcagcccttcatgaggcctgtgcagaccacacaggaggaggacggctgctcctgcaggttccctgaggaggaggagggaggctgcgagctgtggaggaggaagagaaaggagaagcagtccgagacctcccccaaggagttcctcaccatttacgaggacgtgaaggacctgaagaccaggagaaaccacgagcaggaacaaaccttccccggcggcggcagcaccatctacagcatgatccagagccagtcctccgcccctacaagccaggagcctgcctacaccctgtacagcctgatccagcctagcaggaagagcggctccaggaagaggaaccactcccccagcttcaacagcaccatttatgaggtgatcggcaagtcccagcccaaggcccagaaccctgccagactgtccaggaaggagctggagaacttcgacgtctactccggcggcggcggcagcggcggaggaggctccggaggaggcggcagcatgcaggatgaggacggctatatgaccctgaacgtccagtccaagaagaggtccagcgctcagaccagccagctgaccttcaaggactactccgtgaccctgcactggtacaagtgagcggccgcgtcgac

SEQ ID NO: 49 - DNA, 989 bp, Homo sapiens
  misc_feature 1..989: note = NK15_7
  source 1..989: mol_type = other DNA
ggatccgaattcgccgccaccatggccctgcctgtgacagccctgctgctgcctctggctctgctgctgcatgccgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgacatctacatctgggcccctctggccggcacatgtggagtgctgctgctgtccctggtgatcaccaagaggggcaggaagaagctgctgtacatcttcaagcagcccttcatgaggcccgtgcagaccacccaggaggaggacggctgctcctgcagattccccgaggaggaggagggcggatgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 50 - DNA, 1430 bp, Homo sapiens
  misc_feature 1..1430: note = NK15_8
  source 1..1430: mol_type = other DNA
ggatccgaattcgccgccaccatggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgatgggccagcctggcaacggcagcgcctttctgctggcccccaacaggagccatgcccctgaccacgacgtgacccagcagagggacgaggtgtgggtggtgggcatgggcatcgtgatgagcctgatcgtgctggccatcgtgttcggcaacgtgctggtgatcaccgccatcgccaagttcgagaggaagaggggcaggaagaagctgctgtacatcttcaagcagcccttcatgagacccgtgcaaaccacccaggaggaggacggctgcagctgcaggtttcccgaggaggaggagggcggatgcgaactgggaggcggaggaagcggaggaggaggatccggaggaggcggaagcatgcaggacgaggacggctacatgaccctgaacgtccagagcaagaagaggagcagcgcccagacctcccagctgaccttcaaggactactccgtgaccctgcactggtacaagtgagcggccgcgtcgac

SEQ ID NO: 51 - DNA, 1439 bp, Homo sapiens
  misc_feature 1..1439: note = NK15_9
  source 1..1439: mol_type = other DNA
ggatccgaattcgccgccaccatggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgacatctacatctgggcccctctggccggcacatgtggagtgctgctgctgtccctggtgatcaccaagaggggcaggaagaagctgctgtacatcttcaagcagcccttcatgaggcccgtgcagaccacccaggaggaggacggctgctcctgcagattccccgaggaggaggagggcggatgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 52 - DNA, 1329 bp, Homo sapiens
  misc_feature 1..1329: note = NK15_10
  source 1..1329: mol_type = other DNA
gccgccacaatggccctgcctgtgacagccctgctgctgcctctggccctgctgctgcatgctgccaggcctctgttcaaccaggaggtgcagatccctctgaccgagagctactgcggcccctgccccaagaactggatctgctacaagaacaactgctaccagttcttcgacgagagcaagaactggtacgagagccaggccagctgcatgtcccagaacgctagcctgctgaaggtgtatagcaaggaggaccaggacctgctgaagctggtgaagagctaccactggatgggcctggtgcacatccccaccaacggctcctggcagtgggaggacggcagcatcctgagccctaacctgctgaccatcatcgagatgcagaagggagactgcgccctgtacgccagctcctttaagggctacatcgagaactgcagcacccccaacacctacatctgtatgcagaggaccgtgggaggcggcggcagcggcggcggcggcagcggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgcccctagaccccctacacctgcccctaccatcgccagccagcctctgagcctgagacccgaggcctgtagacctgctgccggaggagccgtgcacacaagaggcctggacttcgcctgcgacgtgagcttctgcctggtgatggtgctgctgttcgccgtggacaccggcctgtacttcagcgtgaagaccaacatcaggagcagcaccagggactggaaggaccacaaattcaagtggaggaaggacccccaggacaagaagaggggcaggaagaagctgctgtacatcttcaagcagcccttcatgaggcctgtgcagaccacccaggaggaggacggctgcagctgcaggttccctgaggaggaagagggcggctgcgagctgtga

SEQ ID NO: 53 - DNA, 1239 bp, Homo sapiens
  misc_feature 1..1239: note = NK15_11
  source 1..1239: mol_type = other DNA
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagctga

SEQ ID NO: 54 - DNA, 1064 bp, Homo sapiens
  misc_feature 1..1064: note = NK15_12
  source 1..1064: mol_type = other DNA
ggatccgaattcgccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 55 - protein, 8 aa, Homo sapiens
  REGION 1..8: note = MISC_FEATURE - FLAG tag
DYKDDDDK

SEQ ID NO: 56 - protein, 6 aa, Homo sapiens
  REGION 1..6: note = MISC_FEATURE - His tag
HHHHHH

SEQ ID NO: 57 - protein, 10 aa, Homo sapiens
  REGION 1..10: note = MISC_FEATURE - Myc tag
EQKLISEEDL

SEQ ID NO: 58 - DNA, 1499 bp, Homo sapiens
  misc_feature 1..1499: note = DNA Sequence for Variant 13
  source 1..1499: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcta

SEQ ID NO: 59 - protein, 499 aa, Homo sapiens
  REGION 1..499: note = MISC_FEATURE - Amino Acid Sequence for Variant 13
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 870 bp, Homo sapiens
  misc_feature 1..870: note = DNA Sequence Variant 14
  source 1..870: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgctgtgcgcacgcccacgccgcagccccgcccaagatggcaaagtctacatcaacatgccaggcaggggc

SEQ ID NO: 61 - protein, 290 aa, Homo sapiens
  REGION 1..290: note = MISC_FEATURE - Amino Acid Sequence Variant 14
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLCARPRRSPAQDGKVYINMPGRG

SEQ ID NO: 62 - DNA, 1230 bp, Homo sapiens
  misc_feature 1..1230: note = DNA Sequence Variant 15
  source 1..1230: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgctgtgcgcacgcccacgccgcagccccgcccaagatggcaaagtctacatcaacatgccaggcaggggctggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagc

SEQ ID NO: 63 - protein, 410 aa, Homo sapiens
  REGION 1..410: note = MISC_FEATURE - Amino Acid Sequence Variant 15
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLCARPRRSPAQDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 64 - DNA, 1232 bp, Homo sapiens
  misc_feature 1..1232: note = DNA Sequence Variant 16
  source 1..1232: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagcctgtgcgcacgcccacgccgcagccccgcccaagatggcaaagtctacatcaacatgccaggcaggggctg

SEQ ID NO: 65 - protein, 410 aa, Homo sapiens
  REGION 1..410: note = MISC_FEATURE - Amino Acid Sequence Variant 16
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQDGKVYINMPGRG

SEQ ID NO: 66 - DNA, 1587 bp, Homo sapiens
  misc_feature 1..1587: note = DNA Sequence Variant 17
  source 1..1587: mol_type = other DNA
atggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 67 - protein, 529 aa, Homo sapiens
  REGION 1..529: note = MISC_FEATURE - Amino Acid Sequence Variant 17
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 405 bp, Homo sapiens
  misc_feature 1..405: note = DNA Sequence for alternative NKG2D codon-optimized extracellular domain
  source 1..405: mol_type = other DNA
ctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtg

SEQ ID NO: 69 - protein, 21 aa, Homo sapiens
  REGION 1..21: note = MISC_FEATURE - Amino Acid Sequence CD3zeta transmembrane
LCYLLDGILFIYGVILTALFL

SEQ ID NO: 70 - DNA, 876 bp, Homo sapiens
  misc_feature 1..876: note = DNA Sequence Variant 18 (NK39)
  source 1..876: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatcccaaactctgctacctgctggatggaatcctcttcatctatggtgtcattctcactgccttgttcctgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 71 - protein, 291 aa, Homo sapiens
  REGION 1..291: note = MISC_FEATURE - Amino Acid Sequence Variant 18 (NK39)
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLKTNIRSSTRDWKDHKFKWRKDPQDKKRGRKKLLYIFKQPFMRPVQTTQEEDGCSCRFPEEEEGGCEL

SEQ ID NO: 72 - DNA, 1323 bp, Homo sapiens
  misc_feature 1..1323: note = DNA Sequence NK39_1
  source 1..1323: mol_type = other DNA
atggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgaccccaaactctgctacctgctggatggaatcctcttcatctatggtgtcattctcactgccttgttcctgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactg

SEQ ID NO: 73 - protein, 441 aa, Homo sapiens
  REGION 1..441: note = MISC_FEATURE - Amino Acid Sequence NK39_1
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLKTNIRSSTRDWKDHKFKWRKDPQDKKRGRKKLLYIFKQPFMRPVQTTQEEDGCSCRFPEEEEGGCEL

SEQ ID NO: 74 - DNA, 1032 bp, Homo sapiens
  misc_feature 1..1032: note = DNA Sequence NK39_2
  source 1..1032: mol_type = other DNA
atggccctgcccgtgacagctctgctgctgcctctggccctgctgctgcatgccgctagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgaccccaaactctgctacctgctggatggaatcctcttcatctatggtgtcattctcactgccttgttcctgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaag

SEQ ID NO: 75 - protein, 344 aa, Homo sapiens
  REGION 1..344: note = MISC_FEATURE - Amino Acid Sequence NK39_2
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLKTNIRSSTRDWKDHKFKWRKDPQDKKRGRKKLLYIFKQPFMRPVQTTQEEDGCSCRFPEEEEGGCELGGGGSGGGGSGGGGSMQDEDGYMTLNVQSKKRSSAQTSQLTFKDYSVTLHWYK

SEQ ID NO: 76 - DNA, 1416 bp, Homo sapiens
  misc_feature 1..1416: note = DNA Sequence NK39_3
  source 1..1416: mol_type = other DNA
atggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgaccccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaag

SEQ ID NO: 77 - protein, 472 aa, Homo sapiens
  REGION 1..472: note = MISC_FEATURE - Amino Acid Sequence NK39_3
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCELGGGGSGGGGSGGGGSMQDEDGYMTLNVQSKKRSSAQTSQLTFKDYSVTLHWYK

SEQ ID NO: 78 - DNA, 807 bp, Homo sapiens
  misc_feature 1..807: note = DNA Sequence NK39_4
  source 1..807: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatcccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactg

SEQ ID NO: 79 - protein, 269 aa, Homo sapiens
  REGION 1..269: note = MISC_FEATURE - Amino Acid Sequence NK39_4
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCEL

SEQ ID NO: 80 - DNA, 1143 bp, Homo sapiens
  misc_feature 1..1143: note = DNA Sequence NK39_5
  source 1..1143: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatcccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 81 - protein, 381 aa, Homo sapiens
  REGION 1..381: note = MISC_FEATURE - Amino Acid Sequence NK39_5
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 965 bp, Homo sapiens
  misc_feature 1..965: note = DNA Sequence NK39_6
  source 1..965: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatcccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaa

SEQ ID NO: 83 - protein, 322 aa, Homo sapiens
  REGION 1..322: note = MISC_FEATURE - Amino Acid Sequence NK39_6
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCELGGGGSGGGGSGGGGSMQDEDGYMTLNVQSKKRSSAQTSQLTFKDYSVTLHWYK

SEQ ID NO: 84 - DNA, 927 bp, Homo sapiens
  misc_feature 1..927: note = DNA Sequence NK39_7
  source 1..927: mol_type = other DNA
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatcccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcaagacaaatatcaggtctagcacccgcgactggaaggatcacaagtttaagtggcggaaggaccctcaggataag

SEQ ID NO: 85 - protein, 309 aa, Homo sapiens
  REGION 1..309: note = MISC_FEATURE - Amino Acid Sequence NK39_7
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCELGGGGSGGGGSGGGGSKTNIRSSTRDWKDHKFKWRKDPQDK

SEQ ID NO: 86 - DNA, 933 bp, Homo sapiens
  misc_feature 1..933: note = DNA Sequence NK39_8
  source 1..933: mol_type = other DNA
atggccctgcccgtgacagctctgctgctgcctctggccctgctgctgcatgccgctagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgaccccaaactctgctacctgctggatggaatcctcttcatctatggtgtcattctcactgccttgttcctgctttactgcaagcggggcagaaagaagctgctgtatatcttcaagcagcccttcatgcggcccgtgcagacaacccaggaggaagacggctgctcatgtagatttcctgaagaagaagaagggggctgtgaactgcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 87 - protein, 311 aa, Homo sapiens
  REGION 1..311: note = MISC_FEATURE - Amino Acid Sequence NK39_8
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 88 - DNA, 1605 bp, Homo sapiens
  misc_feature 1..1605: note = DNA Sequence NK39_9
  source 1..1605: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccggcggaggaggcagcggcggcggcggcagcggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 89 - protein, 535 aa, Homo sapiens
  REGION 1..535: note = MISC_FEATURE - Amino Acid Sequence NK39_9
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1122 bp, Homo sapiens
  misc_feature 1..1122: note = DNA Sequence NKG2D-Ox40-CD3z
  source 1..1122: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 91 - protein, 341 aa, Homo sapiens
  REGION 1..341: note = MISC_FEATURE - Amino Acid Sequence NKG2D-OX40-CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1143 bp, Homo sapiens
  misc_feature 1..1143: note = DNA Sequence NKG2D-CD28 - CD3z
  source 1..1143: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 93 - protein, 381 aa, Homo sapiens
  REGION 1..381: note = MISC_FEATURE - Amino Acid Sequence NKG2D-CD28 - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 1269 bp, Homo sapiens
  misc_feature 1..1269: note = DNA Sequence NKG2D - CD28 - 41BB - CD3z
  source 1..1269: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 95 - protein, 423 aa, Homo sapiens
  REGION 1..423: note = MISC_FEATURE - Amino Acid Sequence NKG2D - CD28 - 41BB - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 1038 bp, Homo sapiens
  misc_feature 1..1038: note = DNA Sequence NKG2D(short hinge) - 41BB - CD3z
  source 1..1038: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtggagtccaaatatggtcccccatgcccatcatgcccaatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 97 - protein, 346 aa, Homo sapiens
  REGION 1..346: note = MISC_FEATURE - Amino Acid Sequence NKG2D(short hinge) - 41BB - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - DNA, 1044 bp, Homo sapiens
  misc_feature 1..1044: note = DNA Sequence NKG2D (SH)-CD28 - CD3z
  source 1..1044: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtggagtccaaatatggtcccccatgcccatcatgcccattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 99 - protein, 348 aa, Homo sapiens
  REGION 1..348: note = MISC_FEATURE - Amino Acid Sequence NKG2D (SH)-CD28 - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1023 bp, Homo sapiens
  misc_feature 1..1023: note = DNA Sequence NKG2D (SH) - OX40 - CD3z
  source 1..1023: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtggagtccaaatatggtcccccatgcccatcatgcccaatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 101 - protein, 346 aa, Homo sapiens
  REGION 1..346: note = MISC_FEATURE - Amino Acid Seqeunce NKG2D (SH) - OX40 - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPFWVLVVVGGVLACYSLLVTVAFIIFWVRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1125 bp, Homo sapiens
  misc_feature 1..1125: note = DNA Sequence NKG2D-CD3TM -CD28 - CD3z
  source 1..1125: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatcccaaactctgctacctgctggatggaatcctcttcatctatggtgtcattctcactgccttgttcctgaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 103 - protein, 375 aa, Homo sapiens
  REGION 1..375: note = MISC_FEATURE - Amino Acid Sequence NKG2D-CD3TM -CD28 - CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDPKLCYLLDGILFIYGVILTALFLKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 36 bp, Homo sapiens
  misc_feature 1..36: note = DNA Sequence IgG 4 hinge
  source 1..36: mol_type = genomic DNA
gagtccaaatatggtcccccatgcccatcatgccca

SEQ ID NO: 105 - protein, 29 aa, Homo sapiens
  REGION 1..29: note = MISC_FEATURE - Amino Acid Sequence CD28 Transmembrane domain
FWVLVVVGGVLACYSLLVTVAFIIFWVRS

SEQ ID NO: 106 - protein, 39 aa, Homo sapiens
  REGION 1..39: note = MISC_FEATURE - Amino Acid Sequence CD28 IC domain
KRSRLLHSDYMNMTPRRPGPTRKHYQPYAPPRDFAAYRS

SEQ ID NO: 107 - protein, 37 aa, Homo sapiens
  REGION 1..37: note = MISC_FEATURE - Amino Acid Sequence OX40 IC Domain
RRDQRLPPDAHKPPGGGSFRTPIQEEQADAHSTLAKI

SEQ ID NO: 108 - DNA, 1140 bp, Homo sapiens
  misc_feature 1..1140: note = NKG2D-P-frag/CD8a/4-1BB/CD3z
  source 1..1140: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 109 - protein, 374 aa, Homo sapiens
  REGION 1..374: note = MISC_FEATURE - NKG2D-V2-OX40-CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR